(19) United States
(12) Patent Application Publication
(10) Pub. No.: US 2024/0325326 A1
(43) Pub. Date: Oct. 3, 2024

(54) METHOD OF TREATING PULMONARY FIBROSIS BY TARGETING GLYCOGEN UTILIZATION

(71) Applicant: University of Kentucky Research Foundation, Lexington, KY (US)

(72) Inventors: Matthew S. Gentry, Lexington, KY (US); Ramon C. Sun, Lexington, KY (US); Ronald C Bruntz, Lexington, KY (US); Christopher M Waters, Lexington, KY (US)

(21) Appl. No.: 18/583,392

(22) Filed: Feb. 21, 2024

Related U.S. Application Data
(60) Provisional application No. 63/486,086, filed on Feb. 21, 2023.

(51) Int. Cl. A61K 31/167; A61K 31/166; A61K 31/404; A61K 31/4155; A61K 31/4178; A61K 31/4184; A61K 31/426; A61K 31/428; A61K 31/433; A61K 31/436; A61K 31/47; A61K 31/5375; A61P 11/00

(52) U.S. Cl. CPC A61K 31/167; A61K 31/166; A61K 31/404; A61K 31/4155; A61K 31/4178; A61K 31/4184; A61K 31/426; A61K 31/428; A61K 31/433; A61K 31/436; A61K 31/47; A61K 31/5375; A61P 11/00; C07K 2317/569

(57) ABSTRACT

A method for treating pulmonary fibrosis (PF) is provided. The method includes administering to a subject in need thereof an effective amount of a compound selected from the group consisting of a glycogen phosphatase inhibitor, an acid alpha-glucosidase (GAA) inhibitor, a glycogen synthase (GYS) inhibitor, a glycogen phosphorylase (GP) inhibitor, and combinations thereof, or a pharmaceutically acceptable salt thereof.

### CROSS-REFERENCE TO RELATED APPLICATION

The present application claims priority to U.S. Patent Application Ser. No. 63/486,086, filed on Feb. 21, 2023, the entire disclosure of which is incorporated herein by reference.

### GOVERNMENT INTEREST

This invention was made with government support under grant number R35NS116824 awarded by the National Institutes of Health. The government has certain rights in the invention.

### REFERENCE TO AN ELECTRONIC SEQUENCE LISTING

The contents of the electronic sequence listing (Name: 041824_Sequence Listing_UKRF 2672US.xml; Size: 4,681 bytes; and Date of Creation: Apr. 18, 2024) is herein incorporated by reference in its entirety.

### TECHNICAL FIELD

The presently disclosed subject matter generally relates to treatment of pulmonary fibrosis (PF). In particular, certain embodiments of the presently disclosed subject matter relate to methods of treatment that involve inhibiting glycogen utilization in a subject.

### INTRODUCTION

Pulmonary fibrosis (PF) is an incurable disease that impacts hundreds of thousands of patients. For PF patients without the possibility of a lung transplant, the only available treatment options are anti-inflammatory agents, which only modestly delay disease progression.

Accordingly, there remains a need in the art for improved treatments for PF.

### SUMMARY

The presently disclosed subject matter meets some or all of the above-identified needs, as will become evident to those of ordinary skill in the art after a study of information in this document.

This Summary describes several embodiments of the presently disclosed subject matter and, in many cases, lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features or all embodiments disclosed herein.

The presently disclosed subject matter includes a method for treating pulmonary fibrosis (PF) in a subject in need thereof. In some embodiments, the method includes administering to the subject an effective amount of a compound selected from the group consisting of a glycogen phosphatase inhibitor, an acid alpha-glucosidase (GAA) inhibitor, a glycogen synthase (GYS) inhibitor, a glycogen phosphorylase (GP) inhibitor, and combinations thereof, or a pharmaceutically acceptable salt thereof.

In some embodiments, the method includes administering a glycogen phosphatase inhibitor to the subject. In some embodiments, the glycogen phosphatase inhibitor is a laforin inhibitor. In some embodiments, the laforin inhibitor is of the formula selected from the group consisting of

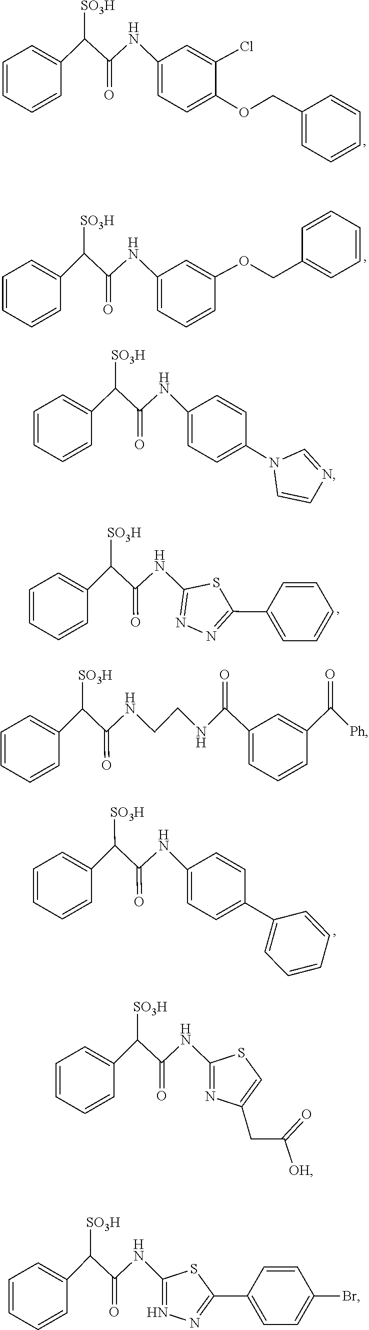

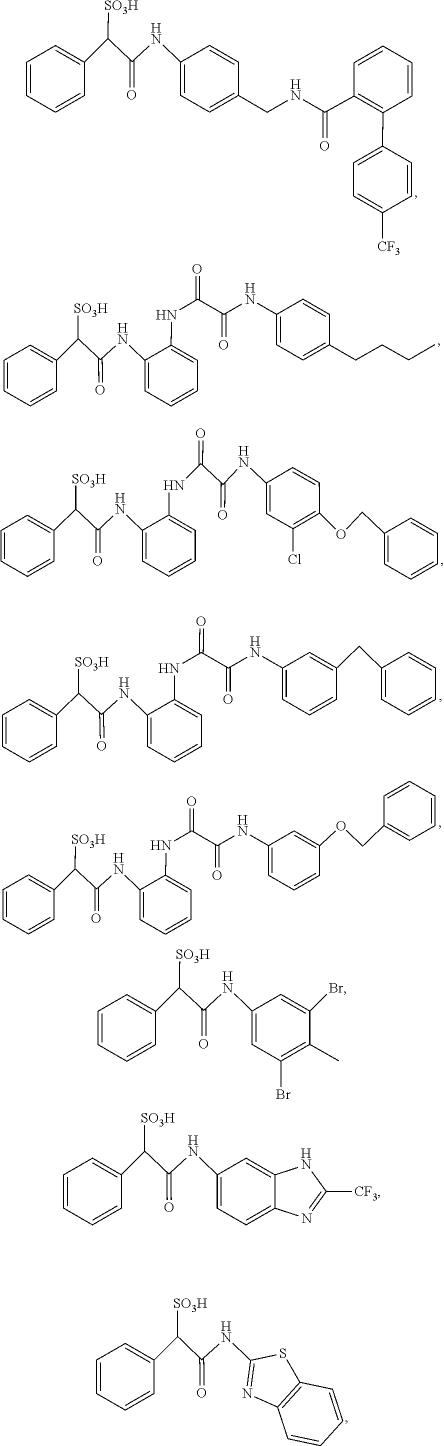

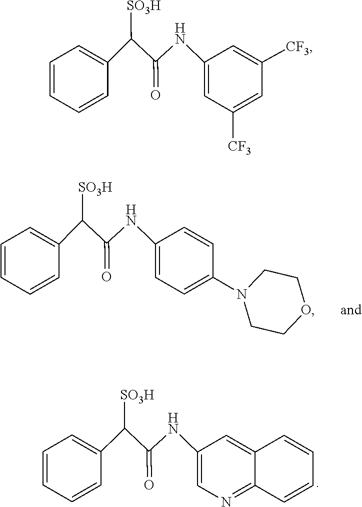 [SMILES: O=C(Nc1cc(C(F)(F)F)cc(C(F)(F)F)c1)C(c1ccccc1)S(=O)(=O)O ; O=C(Nc1ccc(N2CCOCC2)cc1)C(c1ccccc1)S(=O)(=O)O ; O=C(Nc1cnc2ccccc2c1)C(c1ccccc1)S(=O)(=O)O]

In some embodiments, the laforin inhibitor is of the formula of

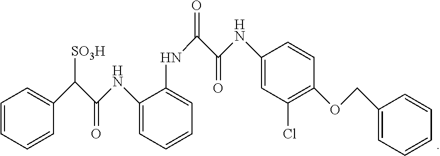 [SMILES: O=C(Nc1ccc(OCc2ccccc2)c(Cl)c1)C(=O)Nc1ccccc1NC(=O)C(c1ccccc1)S(=O)(=O)O]

In some embodiments the laforin inhibitor is a nanobody comprising the sequence of SEQ ID NO: 1, SEQ ID NO: 2, and SEQ ID NO: 3.

In some embodiments, the method includes administering a GAA inhibitor to the subject. In some embodiments, the GAA inhibitor is selected from the group consisting of acarbose, miglitol, voglibose, castanospermine, miglustat, and 1-deoxynojirimycin.

In some embodiments, the method includes administering a GYS inhibitor to the subject. In some embodiments, the GYS inhibitor is of the formula of:

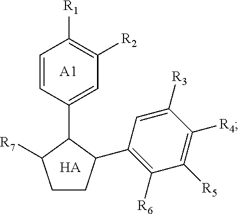

wherein HA is selected from the group consisting of

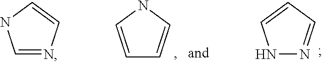

wherein A₁ is selected from the group consisting of and

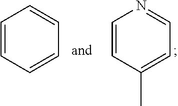

wherein R₁ is selected from the group consisting of OH, H, CONH₂, F, Cl,

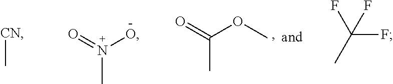

wherein R₂ is selected from the group consisting of OCH₃, H, and Cl;
wherein R₃ is selected from the group consisting of H and F;
wherein R₄ is selected from the group consisting of H, OCH₃, and OH;
wherein R₅ and R₆ are independently selected from the group consisting of H and OH; and
wherein R₇ is selected from the group consisting of

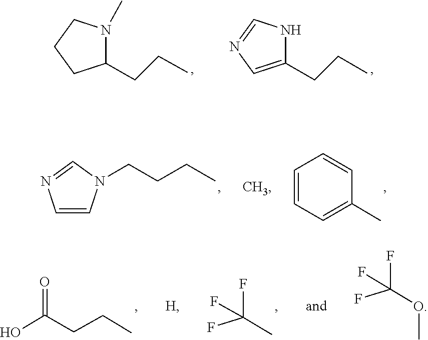

In some embodiments, the GYS inhibitor is of the formula selected from the group consisting of

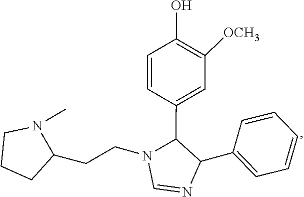 [SMILES: COc1cc(C2C(c3ccccc3)N=CN2CCC2CCCN2C)ccc1O]

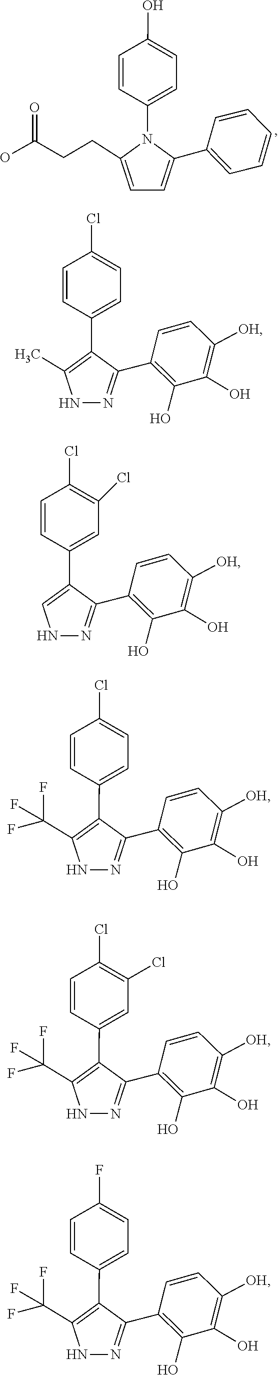

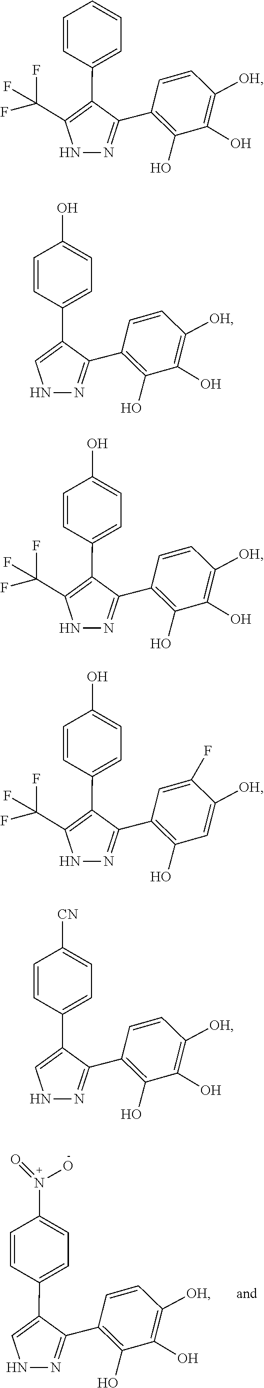

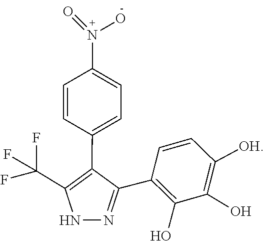

In some embodiments, the GYS inhibitor is of the formula of

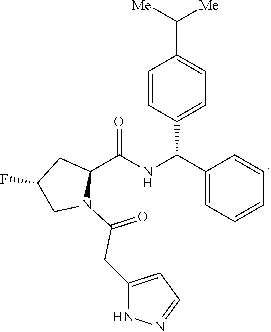 [SMILES: CC(C)c1ccc([C@@H](NC(=O)[C@@H]2C[C@@H](F)CN2C(=O)Cc2ccn[nH]2)c2ccccc2)cc1]

In some embodiments, the GYS inhibitor is guaiacol.

In some embodiments, the method includes administering a GP inhibitor to the subject. In some embodiments, the GP inhibitor is of the formula selected from the group consisting of

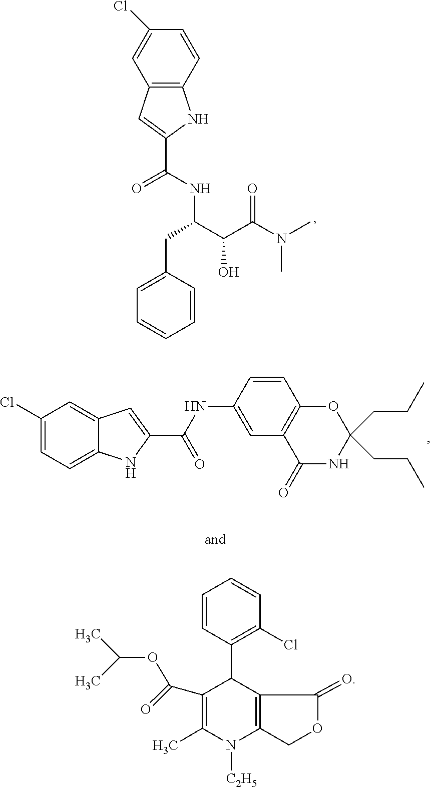

In some embodiments, the subject is identified as being at risk for PF. In some embodiments, the subject is identified as having PF. In some embodiments, the subject has at least one of a history of smoking and a family history of PF.

Further features and advantages of the presently disclosed subject matter will become evident to those of ordinary skill in the art after a study of the description, figures, and non-limiting examples in this document.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIGS. 1A-1K. Matrix-Assisted Laser Desorption/Ionization Mass Spectrometry Imaging (MALDI-MSI) of complex carbohydrates is a multidimensional dataset. FIG. 1A shows a schematic of metabolic pathways of complex carbohydrate metabolism inside a cell. Anabolic metabolism of glycogen and N-linked glycans through the endoplasmic reticulum (ER) and Golgi are critical for a multitude of cellular functions. FIG. 1B shows a schematic of the workflow for multiplexed imaging of glycogen and N-glycans using formalin-fixed paraffin-embedded (FFPE) specimens. Tissues are sectioned onto a microscope slide and are co-treated with peptide: N-glycosidase F (PNGase F) to cleave and release N-glycans and isoamylase to cleave α-1,6-glycosidic bonds releasing linear oligosaccharide chains. Following application of α-cyano-4-hydroxycinnamic acid (CHCA) ionization matrix, samples are analyzed by MALDI and quadrupole time-of-flight mass spectrometry. FIG. 1C shows a schematic of molecular ions recorded in each pixel after laser desorption ionization (left) and a spatial heatmap of a specific biological feature (m/z) extrapolated from total ion current (TIC) after MALDI-MSI of tissue sections (right). FIG. 1D shows TIC representing the sum of all pixels after MALDI-MSI of a human liver section. FIG. 1E shows spatial heatmap images of selected complex carbohydrate ions (m/z) corresponding to either glycogen or N-linked glycans selected from FIG. 1D. m/z values and molecular structures of selected oligosaccharide chain and N-linked glycans are on top of the heatmap. An adjacent liver section stained with hematoxylin and eosin (H&E) is presented below the heatmap for 1743 m/z. Scale bar: 2 mm. FIG. 1F shows TIC extracted from three unique pixels based on the spatial heatmap presented in FIG. 1E. FIG. 1G shows a simplified flow chart for the high-dimensionality reduction and spatial clustering (HDR-SC) analysis workflow. The user input module includes MALDI-MSI and data curation, followed by the computer-based module that utilizes unsupervised clustering and spatial annotation of unique clusters. FIG. 1H shows representative input data for HDR-SC presented as heatmap to show heterogeneous complex carbohydrate abundance. 5% of total pixels (columns) and mixture of matrix and carbohydrate m/z (rows) were shown as heatmap for ease of visualization. FIG. 1I shows UMAP and spatial plots of the human liver specimen by HDR-SC analysis. FIG. 1J shows UMAP and spatial plots showing matrix only cluster from the human liver specimen by HDR-SC analysis. FIG. 1K shows UMAP and spatial plots after matrix removal from the human liver specimen shown in FIG. 1I.

FIGS. 2A-2L. High-dimensionality reduction and spatial clustering (HDR-SC) analysis reveals liver tissue architecture and metabolism. FIG. 2A shows a schematic of isoamylase digestion of glycogen to produce free linear oligosaccharide chains. FIG. 2B shows a schematic of peptide: N-glycosidase F (PNGase F) release of free N-linked glycans from glycoproteins. FIG. 2C shows a schematic of ion mobility separation of linear oligosaccharide chains and N-linked glycans during MALDI-MSI. FIG. 2D shows representative unprocessed and combined ion spectra showing mass drift during MALDI-MSI (left) and spatial heatmap of representative linear oligosaccharide and N-glycans using unprocessed m/z (Right). Scale bar: 2 mm. FIG. 2E shows representative combined ion spectra post processing accounting for mass drift during MALDI-MSI (left) and spatial heatmap of representative linear oligosaccharide and N-glycans using post processed m/z (right). Scale bar: 2 mm. FIG. 2F is an image showing hematoxylin and eosin (H&E) staining of an immediate adjacent section used for MALDI-MSI for histopathology assessment. FIG. 2G shows all identified clusters visualized by UMAP (left) and spatial plots (right) based on pixel coordinate information. FIG. 2H shows zoomed in images of H&E staining of liver sections showing liver lobules, smooth muscle, and portal veins. FIG. 2I shows UMAP (left) and spatial (right) plots highlighting inner liver lobule (cluster 6), outer liver lobule (cluster 1), smooth muscle (cluster 0), and portal vein (cluster 12). FIG. 2J is a table showing spatial clusters to histopathology by a panel of board-certified pathologists. FIG. 2K shows schematics of liver architecture regions (top) and representative spatial distribution and heatmap of a single carbohydrate feature for each region based on differential expression analysis (bottom). Molecular structure and corresponding m/z (rounded to the nearest whole number) of each complex carbohydrate feature were shown below the spatial heatmap. Scale bar: 2 mm. FIG. 2L is a chart showing glycogen chain length abundance and distribution based on UMAP clusters between inner liver lobule (cluster 6), outer liver lobule (cluster 1), smooth muscle (cluster 0), and portal vein (cluster 12). Values are presented as mean+/−standard error. n=3 technical replicates per cluster.

FIG. 3A-3O. High-dimensionality reduction and spatial clustering (HDR-SC) analysis of tissue sections from idiopathic pulmonary fibrosis (IPF) and COVID-19 patients. FIG. 3A is an image showing hematoxylin and eosin (H&E) staining of an immediate adjacent IPF section used for MALDI-MSI for histopathology assessment. Scale bar: 5 mm. FIG. 3B shows all identified clusters visualized by UMAP (left) and spatial plots (right) based on pixel coordinate information in an immediate adjacent IPF section. FIG. 3C is a table showing spatial clusters to histopathology by a panel of board-certified pathologists in an immediate adjacent IPF section. FIG. 3D shows zoomed in images of H&E staining of IPF section showing diffuse alveolar damage (DAD), end-stage fibrosis, and mucin aggregates in an immediate adjacent IPF section. Scale bar: 500 μm. FIG. 3E shows UMAP (left) and spatial (right) plots highlighting DAD, end-stage fibrosis, and mucin aggregate clusters. FIG. 3F is an image showing H&E staining of an immediate adjacent COVID-19 section used for MALDI-MSI for histopathology assessment. Scale bar: 5 mm. FIG. 3G shows all identified clusters visualized by UMAP (left) and spatial plots (right) based on pixel coordinate information in an immediate adjacent COVID-19 section. FIG. 3H is a table showing spatial clusters to histopathology by a panel of board-certified pathologists in an immediate adjacent COVID-19 section. FIG. 3I shows zoomed in images of H&E staining of COVID-19 section showing DAD, end-stage fibrosis, and mucin aggregates in an immediate adjacent COVID-19 section. Scale bar: 500 μm. FIG. 3J shows UMAP (left) and spatial (right) plots highlighting DAD, end-stage fibrosis, and mucin aggregate clusters. FIG. 3K shows differentially expressed features among HDR clusters, columns represent pixels within each cluster and row is the glycogen or N-glycan feature over-represented. For simplicity, only fibrosis and diffuse alveolar damage were shown. Glycogen (yellow) and core fucosylated N-linked glycan (pink) features were over-represented in fibrotic clusters. FIG. 3L shows a schematic of glycogen accumulation in pulmonary fibrosis. FIG. 3M shows images of H&E staining of additional IPF (n=3, left) and COVID-19 (n=2) tissue sections used for MALDI-MSI for histopathology assessment. Scale bar is below in FIG. 3N. FIG. 3N shows images of spatial distribution and heatmap of glycogen chain length+7 (1175 m/z) in additional patient tissues shown in FIG. 3M. Scale bar: 5 mm. FIG. 3O are images showing immunofluorescent/co-localization analysis of glycogen and alpha smooth muscle actin (α-SMA) from an adjacent 20 μm section of IPF specimen shown in FIG. 3A. Tissue is stained with glycogen (red), α-SMA (green), and DAPI (blue) following by whole slide scanning and visualized using the HALO software. Scale bar: 50 μm. Zoomed out view shown in FIG. 4M.

FIGS. 4A-4N. High-dimensionality reduction and spatial clustering (HDR-SC) analysis of tissue sections from additional idiopathic pulmonary fibrosis (IPF) and COVID-19 patients. FIG. 4A is an image showing hematoxylin and eosin (H&E) staining of an immediate adjacent IPF section used for MALDI-MSI for histopathology assessment. Scale bar: 2 mm. FIG. 4B shows all identified clusters visualized by UMAP (left) and spatial plots (right) based on pixel coordinate information. FIG. 4C is a table showing spatial clusters to histopathology by a panel of board-certified pathologists. FIG. 4D is an image showing H&E staining of an immediate adjacent COVID-19 section used for MALDI-MSI for histopathology assessment. Scale bar: 2 mm. FIG. 4E shows all identified clusters visualized by UMAP (left) and spatial plots (right) based on pixel coordinate information. FIG. 4F is a table showing spatial clusters to histopathology by a panel of board-certified pathologists. FIG. 4G shows a zoomed in image of H&E staining of IPF section showing end-stage fibrosis. Scale bar: 500 μm. FIG. 4H shows UMAP (left) and spatial (right) plots highlighting end-stage fibrosis clusters. FIG. 4I shows zoomed in image of H&E staining of COVID-19 section showing acute fibrinous organizing pneumonia (AFOP) and end-stage fibrosis. Scale bar: 500 μm. FIG. 4J shows UMAP (left) and spatial (right) plots highlighting AFOP and end-stage fibrosis clusters. FIG. 4K shows images of H&E staining of additional IPF (n=3, left) and COVID-19 (n=2) tissue sections used for MALDI-MSI for histopathology assessment. Scale bar is below in FIG. 4L. FIG. 4L shows spatial distribution and heatmap of N-linked glycan 1809 m/z in additional patient tissues shown in (FIG. 4K). Scale bar: 5 mm. FIG. 4M shows images of immunofluorescent/co-localization analysis of glycogen and alpha smooth muscle actin (α-SMA) from an adjacent 20 μm section of IPF specimen shown in FIG. 3A. Tissue is stained with glycogen (red), α-SMA (green), and DAPI (blue) following by whole slide scanning and visualized using the HALO software. Scale bar: 200 μm. Zoomed in field of view (Square) is shown in FIG. 3O. FIG. 4N shows quantitative glycogen MALDI imaging in situ. Increasing amounts of purified glycogen were spotted adjacent to the tissue section and used to generate the standard curve. Absolute glycogen levels in fibrosis and non-fibrotic regions were determined using the equation derived from the line of best fit.

FIGS. 5A-5F. Pulmonary fibrosis patient tissue exhibits increased glycogen accumulation and unique N-linked glycosylation profiles compared to normal lung. FIG. 5A shows images of hematoxylin & Eosin (H&E) staining of the pulmonary fibrosis TMA used for this study with normal (N) and fibrotic (F) tissues annotated. Tissue cores used were further confirmed by pathologist and used in the analysis are highlighted in red. Scale bar: 5 mm. FIG. 5B shows spatial distribution and heatmap of glycogen chain length+7 (1175 m/z) in pulmonary fibrosis TMA shown in FIG. 5A. Scale bar: 5 mm. FIG. 5C shows a spatial distribution and heatmap of N-linked glycan 1809 m/z in pulmonary fibrosis TMA shown in (FIG. 5A). Scale bar: 5 mm. FIG. 5D shows an unsupervised clustering heatmap analysis of all glycogen and N-linked glycan features in normal and fibrosis lung patient samples. FIG. 5E shows a multivariate receiver operating characteristic (ROC) curve of all glycogen features between normal and fibrosis patients. FIG. 5F shows a volcano plot of the fold change between fibrosis/normal lung patient tissue. Glycogen and N-linked glycans features with a fold change>1.5 and P<0.1 are highlighted in red.

FIGS. 6A-6J. Pulmonary fibrosis patient tissue exhibits increased glycogen accumulation and aberrant N-linked glycosylation. FIG. 6A shows a representative spatial distribution and heatmap of glycogen chain length+7 (1175 m/z) in normal (N) and fibrotic (F) lung patient tissue cores from TMA. Scale bar: 2 mm. FIG. 6B is a chart showing total glycogen abundance in normal and fibrotic lung patient tissue measured by MALDI. FIG. 6C is a chart showing glycogen chain length abundance in normal and fibrotic lung patient tissue. FIG. 6D is a plot showing multivariate analysis of glycogen and N-linked glycan features in normal and fibrosis lung patient samples by partial least squares-discriminant analysis (PLS-DA) displaying 95% confidence intervals. FIG. 6E shows an unsupervised clustering heatmap analysis of the top 25 glycogen and N-linked glycan features in normal and fibrotic lung patient. FIG. 6F shows a multivariate receiver operating characteristic (ROC) curve of all glycogen and N-linked glycan features between normal and fibrosis patients. FIG. 6G (left) shows a representative spatial distribution and heatmap of 1809 m/z in N and F lung patient tissue. Scale bar: 2 mm. FIG. 6G (right) shows a relative abundance of 1809 m/z in N and F lung patient tissue. Molecular structure of the selected N-linked glycan is to the right of the graph. FIG. 6H (left) shows a representative spatial distribution and heatmap of 1485 m/z in N and F lung patient tissue. Scale bar: 2 mm. FIG. 6H (right) shows a relative abundance of 1485 m/z in N and F lung patient tissue. Molecular structure of the selected N-linked glycan is to the right of the graph. FIG. 6I (left) shows representative spatial distribution and heatmap of 2012 m/z in N and F lung patient tissue. Scale bar: 2 mm. FIG. 6I (right) shows relative abundance of 2012 m/z in N and F lung patient tissue. Molecular structure of the selected N-linked glycan is to the right of the graph. FIG. 6J (left) shows representative spatial distribution and heatmap of 2122 m/z in N and F lung patient tissue. Scale bar: 2 mm. FIG. 6J (right) shows relative abundance of 2122 m/z in N and F lung patient tissue. Molecular structure of the selected N-linked glycan is to the right of the graph. Values are presented as mean+/−standard error. (Normal: n=7, Fibrosis: n=26). *0.01<P<0.05; **0.001<P<0.01; ***P<0.001; ****P<0.0001, analyzed by two-tailed t-test.

FIGS. 7A-7I. Bleomycin-induced lung fibrosis shares same complex carbohydrate features as human fibrosis. FIG. 7A shows a schematic of bleomycin-induced lung fibrosis model. FIG. 7B shows zoomed in images of hematoxylin and eosin (H&E) staining of lung tissue from control (saline) and bleomycin-treated mice. Scale bar: 200 μm. FIG. 7C (left) shows H&E staining of whole lung tissue from control (saline) (top) and bleomycin-treated (bottom) mice. FIG. 7C (right) shows spatial distribution and heatmap of N-linked glycans: 1809, 2012, and 1485 m/z in lung tissue from control (saline) (top) and bleomycin-treated (bottom) mice. Scale bar: 2 mm. FIG. 7D are charts showing total abundance of N-linked glycans: 1809, 2012, and 1485 m/z in lung tissue from control (saline) and bleomycin-treated mice. Molecular structure of the selected N-linked glycans are above their respective graphs. FIG. 7E (left) shows H&E staining of whole lung tissue from control (saline) (top) and bleomycin-treated (bottom) mice. FIG. 7E (right) shows spatial distribution and heatmap of glycogen chain length+7 (1175 m/z) in lung tissue from control (saline) (top) and bleomycin-treated (bottom) mice. Scale bar: 2 mm. FIG. 7F is a chart showing glycogen chain length abundance in lung tissue from control (saline) and bleomycin-treated mice. FIG. 7G is a chart showing total glycogen abundance in lung tissue from control (saline) and bleomycin-treated mice. Values are presented as mean+/−standard error. (n=3 per group). *0.01<P<0.05; ***P<0.001; ****P<0.0001, analyzed by two-tailed t-test. FIG. 7H shows images of immunofluorescent/co-localization analysis of glycogen and alpha smooth muscle actin (α-SMA) from an adjacent 20 μm section of PF mouse lung shown in FIG. 7C. Tissue is stained with glycogen (red), α-SMA (green), and DAPI (blue) following by whole slide scanning and visualized using the HALO software. Zoomed in view shown below, and the white box in the glycogen panel represents the field of view magnified. Scale bar: 200 μm and 50 μm respectively. FIG. 7I shows schematics of shared glycogen and N-linked glycogen phenotype between mouse and human PF.

FIGS. 8A-8F. Bleomycin-treatment induces aberrant complex carbohydrate metabolism during fibrosis in vivo. FIG. 8A shows images of hematoxylin and eosin (H&E) staining of whole lung tissue from control (saline, left) and bleomycin-treated (right) mice. Scale bar: 2 mm. FIG. 8B shows zoomed in representative images of H&E staining of lung tissue from control (saline, top) and bleomycin-treated (bottom) mice. Scale bar: 200 μm. FIG. 8C is a plot showing multivariate analysis of glycogen and N-linked glycan features in lung tissue from control (saline) and bleomycin-treated mice by partial least squares-discriminant analysis (PLS-DA) displaying 95% confidence intervals. FIG. 8D is a chart showing variable importance in projection (VIP) analysis showing top 15 most discriminant carbohydrate structures revealed by PLS-DA. m/z can be matched to structure in Table 3A and 3B. FIG. 8E shows an unsupervised clustering heatmap analysis of all glycogen and N-linked glycan features in lung tissue from control (saline) and bleomycin-treated mice. FIG. 8F are charts showing targeted liquid-chromatography mass spectrometry analysis of polar metabolites from PBS and bleomycin treated mice.

FIG. 9A-9K. Loss of laforin blunts complex carbohydrate perturbations during bleomycin-induced lung injury. FIG. 9A shows a schematic of bleomycin-induced lung fibrosis model in wild-type (WT) and Epm2a^{-/-} (LKO) mice. FIG. 9B is a chart showing weight change (g) in control (saline) and bleomycin-treated WT and LKO mice during the course of the study. FIG. 9C is a chart showing Kaplan-Meier analysis of overall survival in WT and LKO bleomycin-treated mice. FIG. 9D shows spatial distribution and heatmap of glycogen chain length+7 (1175 m/z) in lung tissue from control (saline) and bleomycin-treated WT and LKO mice. Scale bar: 2 mm. FIG. 9E is a chart showing glycogen chain length abundance in lung tissue from control (saline) and bleomycin-treated WT and LKO mice. FIG. 9F is a chart showing total glycogen abundance in lung tissue from control (saline) and bleomycin-treated WT and LKO mice. FIG. 9G shows zoomed in representative images of hematoxylin and eosin (H&E) staining of lung tissue from control (saline) and bleomycin-treated WT and LKO mice. Scale bar: 200 μm. FIG. 9H is a chart showing Ashcroft scoring for lung tissue from control (saline) and bleomycin-treated WT and LKO mice. FIG. 9I is a chart showing total bronchoalveolar lavage fluid (BALF) collagen (μg/ml) from control (saline) and bleomycin-treated WT and LKO mice. FIG. 9J shows a schematic of MALDI-imaging of collagen peptides using formalin-fixed paraffin-embedded (FFPE) specimens. FIG. 9K (left) shows spatial distribution and heatmap of collagen peptide 1247 m/z in lung tissue from control (saline) and bleomycin-treated WT and LKO mice and total abundance of 1247 m/z in lung tissue from control (saline) and bleomycin-treated WT and LKO mice. FIG. 9K (right) shows spatial distribution and heatmap of collagen peptide 843 m/z in lung tissue from control (saline) and bleomycin-treated WT and LKO mice and total abundance of 843 m/z in lung tissue from control (saline) and bleomycin-treated WT and LKO mice. Putative amino acid sequence, collagen subtype, and # of hydroxylate proline sites (HYP) on are displayed in white text above representative images. Scale bar: 2 mm. Values are presented as mean+/−standard error. *0.01<P<0.05; **0.001<P<0.01; ****P<0.0001, analyzed by one-way ANOVA.

FIGS. 10A-10E. Loss of Laforin blunts bleomycin-induced aberrant complex carbohydrate metabolism in vivo. FIG. 10A shows images of hematoxylin and eosin (H&E) staining of whole lung tissue from control (saline) and bleomycin-treated wild-type (WT) and Epm2a^{-/-} (LKO) mice. Scale bar: 2 mm. FIG. 10B shows images of total collagen peptides detected from WT, LKO, WT-Bleomycin, and Laforin-KO-Bleomycin lung by MALDI-MSI. Ashcroft score is listed below for each set of mouse lungs. Scale bar: 2 mm. FIG. 10C shows spatial distribution and heatmap of collagen peptide 1438 m/z in lung tissue from control (saline) and bleomycin-treated WT and LKO mice and total abundance of 1438 m/z in lung tissue from control (saline) and bleomycin-treated WT and LKO mice. Putative amino acid sequence, collagen subtype, and # of hydroxylate proline sites (HYP) on are displayed on top. Scale bar: 2 mm. FIG. 10D is a plot showing multivariate analysis of collagen peptides in lung tissue from control (saline) and bleomycin-treated WT and Laforin-KO mice by principal component analysis (PCA) displaying 95% confidence intervals. FIG. 10E shows an unsupervised clustering heatmap analysis of all collagen peptides in lung tissue from control (saline) and bleomycin-treated WT and LKO mice.

FIGS. 11A-11H. Increased metabolic demand for glycogen in diseased fibroblasts. FIG. 11A shows images of representative immunohistochemical staining of glycogen synthase (GYS1), glycogen phosphorylate brain isoform (PYGB), glycogen phosphorylate muscle isoform (PYGM) in fibrotic regions and adjacent alveoli structures. Zoomed in images are at the bottom. Scale bar: 200 μm and 50 μm. Arrows represent alveoli lining and fibrosis with GYS1 staining. FIG. 11B shows charts showing quantification of immunohistochemical staining analysis for GYS, PYGB, PYGM abundance in fibrotic regions (n=5) and adjacent normal regions where available (n=3). FIG. 11C (left) shows schematics of patient-derived normal fibroblasts (NF) and disease fibroblasts (DF, isolated from IPF patients). FIG. 11C (right) shows a chart showing relative glycogen levels between NF and DF measured by GCMS (n=3 technical replicates). FIG. 11D shows a schematic of ¹³C-glucose tracing to study glycogen biosynthesis and degradation. FIG. 11E is a chart showing ¹³C-glycogen enrichment from ¹³C-glucose over 48 hours in both NF and DF. FIG. 11F is a chart showing ¹³C-glycogen washout after ¹²C-glucose substitution over 12 hours in both NF and DF. FIG. 11G shows images of hematoxylin and eosin (H&E) staining showing early, mid, and end stage fibrosis in human PF specimens. FIG. 11H shows representative images and relative abundance of glycogen chain length+7 (1175 m/z) and a biantennary glycan spatial distribution in in early-, mid-, and end-stage fibrosis. Molecular structure of the selected N-linked glycan is to the right of the heatmap. Values are presented as mean+/−standard error. *0.01<P<0.05; **0.001<P<0.01; ***P<0.00, analyzed by two-tailed t-test.

FIGS. 12A-12K. Glycogen utilization by lysosomal-GAA during PF in vivo. FIG. 12A shows images of immunofluorescent/co-localization analysis of glycogen and acid alpha-glucosidase (GAA) from an adjacent 20 section of human IPF lung shown in FIG. 3A. Tissue is stained with glycogen (red), GAA (green), and DAPI (blue) following by whole slide scanning and visualized using the HALO software. Scale bar: 10 μm. FIG. 12B shows images of immunofluorescent/co-localization analysis of glycogen and LAMP2 from an adjacent 20 μm section of mouse PF lung (GAA antibody does not work in mouse). Tissue is stained with glycogen (red), LAMP2 (green), and DAPI (blue) following by whole slide scanning and visualized using the HALO software. Scale bar: 20μ. FIG. 12C shows a schematic of lysosomal salvage pathway of glycogen by GAA to provide substrates for other complex carbohydrates. FIG. 12D shows a schematic of bleomycin-induced lung fibrosis model in wild-type (WT) and Gaa^{-/-} mice (GKO). FIG. 12E shows representative images of hematoxylin and eosin (H&E) staining of lung tissue from control (saline) and bleomycin-treated WT and GKO mice. Scale bar: 50 μm. FIG. 12F is a chart showing Ashcroft scoring for lung tissue from control (saline) and bleomycin-treated WT and LKO mice. FIG. 12G is a chart showing glycogen chain length abundance in lung tissue from control (saline) and bleomycin-treated WT and LKO mice. FIG. 12H is a chart showing total glycogen abundance in lung tissue from control (saline) and bleomycin-treated WT and LKO mice. FIG. 12I are charts showing total abundance of 1444 m/z and 2012 m/z in lung tissue from control (saline) and bleomycin-treated WT and GKO mice. Molecular structure of the selected N-linked glycan is to the right of the graph. FIG. 12J are charts showing total abundance of 1444 m/z and 2012 m/z in lung tissue from control (saline) and bleomycin-treated WT and LKO mice. Molecular structure of the selected N-linked glycan is to the right of the graph. FIG. 12K shows a schematic of lysosomal salvaging of glycogen as a critical component of PF progression. Values are presented as mean+/−standard error. *0.01<P<0.05; **0.001<P<0.01, analyzed by one-way ANOVA.

FIGS. 13A-13E. Gaa^{-/-} is protective against bleomycin-induced lung injury in vivo. FIG. 13A shows images of Hematoxylin and eosin (H&E) staining of whole lung tissue from control (saline) and bleomycin-treated wild-type (WT) and Gaa^{-/-} (GKO) mice. Scale bar: 2 mm. FIG. 13B shows images of immunofluorescent/co-localization analysis of glycogen and acid alpha-glucosidase (GAA) from an adjacent 20p section of human IPF lung shown in FIG. 3A. Tissue is stained with glycogen (red), GAA (green), and DAPI (blue) following by whole slide scanning and visualized using the HALO software. Scale bar: 100μ and 50μ respectively. FIG. 13C shows images of Immunofluorescent/co-localization analysis of glycogen and LAMP2 from an adjacent 20p section of human IPF lung shown in FIG. 3A. Tissue is stained with glycogen (red), LAMP2 (green), and DAPI (blue) following by whole slide scanning and visualized using the HALO software. Scale bars: 200μ and 50μ respectively. FIG. 13D shows representative images of H&E staining of lung tissue from control (saline) and bleomycin-treated WT and GKO mice. Scale bar: 200 μm. FIG. 13E shows unsupervised clustering heatmap analysis of all glycogen and N-linked glycan features in lung tissue from control (saline) and bleomycin-treated WT and LKO/GKO mice.

FIG. 14. are charts showing laforin inhibition decreases fibrosis-related gene expression in diseased human lung fibroblasts. Normal human lung fibroblasts (NHLF) or diseased human lung fibroblasts (DHLF) derived from idiopathic pulmonary fibrosis patients were treated for two days with a laforin inhibitor (or vehicle control) before harvesting mRNA and assessing gene expression. Samples were run on Bio-rad Prime PCR Fibrosis gene panel plates with n=3 samples per condition. *p<0.05, ***p<0.001 two-tailed student's t-test. Laforin inhibitor is compound “L319-21-M50” from U.S. Patent Application Publication No 2018/0170862A1, which is incorporated herein by reference in its entirety.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by those of ordinary skill in the art, certain definitions are set forth to facilitate explanation of the presently-disclosed subject matter. Unless defined otherwise, all technical and scientific terms used herein have the same meaning as is commonly understood by one of skill in the art to which the invention(s) belong. All patents, patent applications, published applications and publications, GenBank sequences, databases, websites and other published materials referred to throughout the entire disclosure herein, unless noted otherwise, are incorporated by reference in their entirety. Where reference is made to a URL or other such identifier or address, it is understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently-disclosed subject matter, representative methods, devices, and materials are described herein.

In certain instances, nucleotides and polypeptides disclosed herein are included in publicly-available databases, such as GENBANK® and SWISSPROT. Information including sequences and other information related to such nucleotides and polypeptides included in such publicly-available databases are expressly incorporated by reference. Unless otherwise indicated or apparent the references to such publicly-available databases are references to the most recent version of the database as of the filing date of this Application.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, in some embodiments ±0.1%, in some embodiments ±0.01%, and in some embodiments ±0.001% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

As used herein, the term “treatment” is inclusive of prophylactic treatment and therapeutic treatment. As would be recognized by one of ordinary skill in the art, treatment that is administered prior to clinical manifestation of a condition is prophylactic (i.e., it protects the subject against or reduces the risk of the subject developing the condition). If the treatment is administered after manifestation of the condition, the treatment is therapeutic (i.e., it is intended to diminish, ameliorate, control, or maintain the existing condition and/or side effects associated with the condition). The terms relate to medical management of a subject with the intent to substantially cure, ameliorate, stabilize, or substantially prevent a condition of interest (e.g., disease, pathological condition, or disorder), including but not limited to prophylactic treatment to preclude, avert, obviate, forestall, stop, or hinder something from happening, or reduce the severity of something happening, especially by advance action. As such, the terms treatment or treating include, but are not limited to: inhibiting the progression of a condition of interest; arresting or preventing the development of a condition of interest; reducing the severity of a condition of interest; ameliorating or relieving symptoms associated with a condition of interest; causing a regression of the condition of interest or one or more of the symptoms associated with the condition of interest; and preventing a condition of interest or the development of a condition of interest. The terms includes active treatment, that is, treatment directed specifically toward the improvement of a condition of interest, and also includes causal treatment, that is, treatment directed toward removal of the cause of the condition of interest.

As used herein, the term “effective amount” refers to an amount that is sufficient to achieve the desired prophylactic or therapeutic result or to have an effect on undesired symptoms, but is generally insufficient to cause adverse side effects. The specific effective amount for any particular subject will depend upon a variety of factors including the particular risk or condition being treated and the severity of the condition; the specific composition employed; the age, body weight, general health, sex and diet of the subject; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual medical practitioner in the event of any contraindications. Dosage can vary, and can be administered in one or more dose administrations daily, for one or several days. Guidance can be found in the literature for appropriate dosages for given classes of compounds and products.

As will be recognized by one of ordinary skill in the art, the terms “suppression,” “suppressing,” “suppressor,” “inhibition,” “inhibiting” or “inhibitor” do not refer to a complete elimination of a value in all cases. Rather, the skilled artisan will understand that the term “suppressing” or “inhibiting” refers to a reduction or decrease in a measured value, qualitatively or quantitatively. Such reduction or decrease can be determined relative to a control or a prior status of a subject. In some embodiments, the reduction or decrease relative to a control or the prior status of a subject can be about a 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% decrease.

As used herein, the term “administering” refers to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition. In some embodiments, oral administration is used. In some embodiments, intravenous (IV) administration is used.

As used herein, the term “subject” when referring to a subject having a particular condition or risk thereof, or when referring to a subject in need of treatment for a particular condition or risk thereof, refers to a target of administration, which optionally displays symptoms and/or has risk factures related to the particular condition or risk thereof, as would be considered relevant to one skilled in the medical art of making a diagnosis of the particular condition or risk thereof. As used herein, the term “subject” can refer to a vertebrate, such as a mammal. The term includes human and veterinary subjects. Thus, the subject can be a human, non-human primate, horse, pig, rabbit, dog, sheep, goat, cow, cat, guinea pig, or rodent. The term does not denote a particular age or sex.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, the term “pharmaceutically acceptable salt” when made in reference to a compound refers to a salt that is pharmaceutically acceptable and that possesses the desired pharmacological activity of the parent compound.

The presently disclosed subject matter includes a method for treating pulmonary fibrosis (PF). As disclosed herein, it has been surprisingly discovered that lysosomal utilization of glycogen within a subject is required for PF progression and that certain enzymes contributing to such glycogen utilization, namely, laforin, a glycogen phosphatase, and acid alpha-glucosidase (GAA), can prove as useful targets for treating PF. In view of such discoveries and it being known that glycogen synthase (GYS) (as a catalyzer of glycogen chain elongation by incorporating residues from UDP-glucose to growing glycogen strands) and glycogen phosphorylase (GP) (as a catalyzer of the release of glucose monomers from stored glycogen polymers) also contribute to glycogen synthesis and/or utilization, it is thus believed that GYS and GP may also serve as useful targets for inhibiting glycogen utilization and treating PF.

Accordingly, certain embodiments of the method for treating PF involve inhibiting glycogen utilization within a subject. In some embodiments, glycogen utilization is inhibited within a subject by virtue of administering to the subject an effective amount of a compound selected from the group consisting of a glycogen phosphatase inhibitor, a GAA inhibitor, a GYS inhibitor, a GP inhibitor, and combinations thereof, or a pharmaceutically acceptable salt thereof.

In some embodiments, the method includes administering an effective amount of a glycogen phosphatase inhibitor, or a pharmaceutically acceptable salt thereof, to the subject. As further disclosed below, the inhibition of the glycogen phosphatase laforin has been discovered as being useful in reducing glycogen utilization and blunting fibrosis burden in vivo. Accordingly, in some embodiments, the method involves administering a laforin inhibitor to the subject. Certain laforin inhibitors which are known in the art and which may be utilized in the method for treating PF include those described in U.S. Patent Application Publication No. 2018/0170862 and U.S. Pat. No. 10,532,977, the entire disclosure of each of which is incorporated herein in its entirety by reference. For example, in some embodiments, the laforin inhibitor compound administered to the subject can be

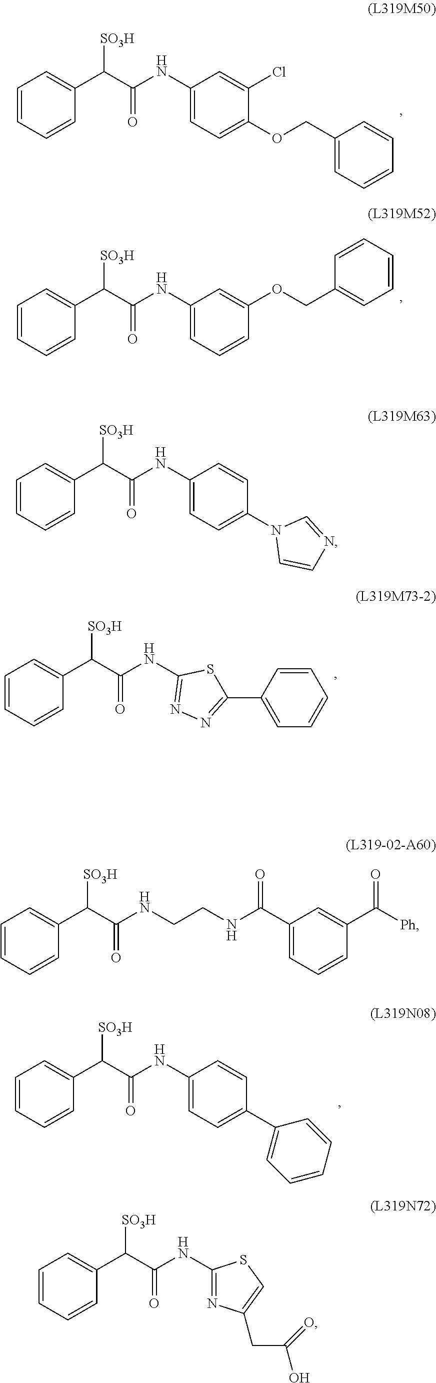

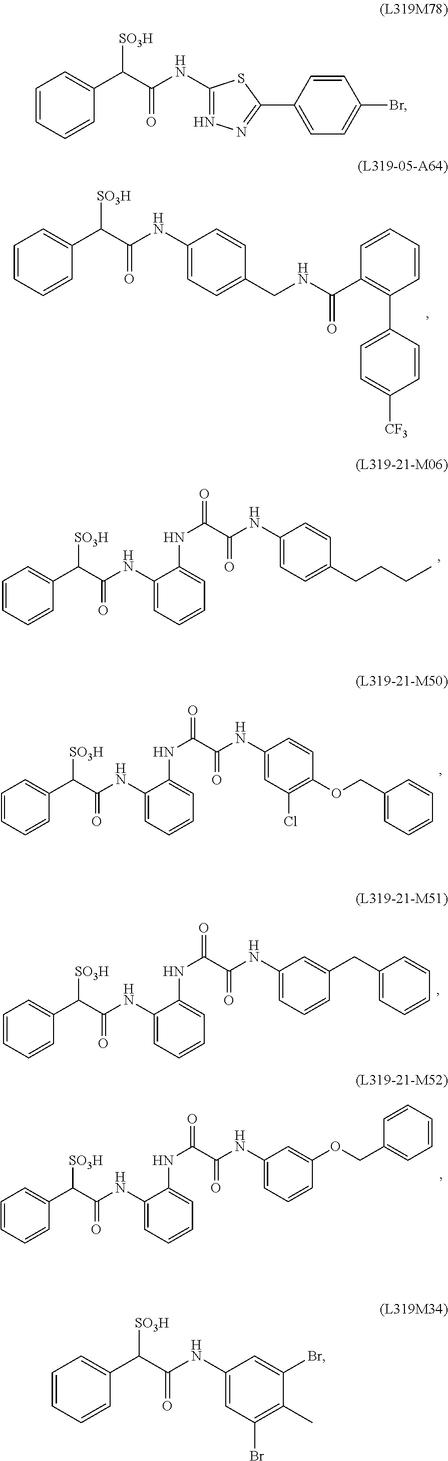

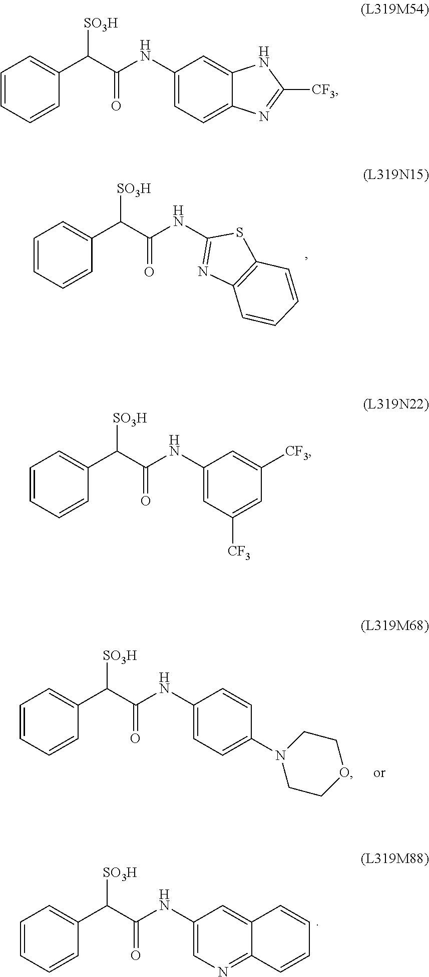 [SMILES: O=C(Nc1ccc2nc(C(F)(F)F)[nH]c2c1)C(c1ccccc1)S(=O)(=O)O ; O=C(Nc1nc2ccccc2s1)C(c1ccccc1)S(=O)(=O)O ; O=C(Nc1cc(C(F)(F)F)cc(C(F)(F)F)c1)C(c1ccccc1)S(=O)(=O)O ; O=C(Nc1ccc(N2CCOCC2)cc1)C(c1ccccc1)S(=O)(=O)O ; O=C(Nc1cnc2ccccc2c1)C(c1ccccc1)S(=O)(=O)O]

In some embodiments, the laforin inhibitor administered to the subject is

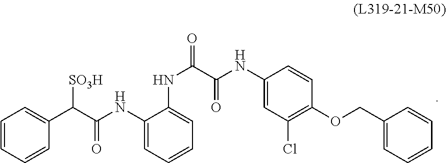 [SMILES: O=C(Nc1ccc(OCc2ccccc2)c(Cl)c1)C(=O)Nc1ccccc1NC(=O)C(c1ccccc1)S(=O)(=O)O]

In some embodiments, the laforin inhibitor may be in the form of a nanobody. In some embodiments, the nanobody comprises the amino acid sequence of INAMA (SEQ ID NO: 1), the amino acid sequence of HISSDNTNYADSVKG (SEQ ID NO: 2), and the amino acid sequence of VATW (SEQ ID NO: 3). In some embodiments, SEQ ID NO: 1, SEQ ID NO: 2, and SEQ ID NO: 3 correspond to a first complementarity determining region (CDR1), a second complementarity determining region (CDR2), and a third complementarity determining region (CDR3), respectively, of the nanobody. One known laforin-inhibiting nanobody which may be utilized as a laforin inhibitor compound administered to the subject is the Nb72 nanobody disclosed in Simmons, Z. R., Sharma, S., Wayne, J., Li, S., Vander Kooi, C. W., and Gentry, M. S. “Generation and characterization of a laforin nanobody inhibitor,” Clinical Biochemistry 93, 80-89 (2021), the entire disclosure of which is incorporated herein by reference. Accordingly, in some embodiments, the nanobody comprises SEQ ID NO: 4. In some embodiments, a combination of glycogen phosphatase inhibitors may be administered to the subject.

As further disclosed below, PF-induced models lacking GAA have been found to exhibit a multi-fold reduction in fibrosis burden relative to controls. Accordingly, in some embodiments, the method involves administering an effective amount a GAA inhibitor, or a pharmaceutically acceptable salt thereof, to the subject. Certain GAA inhibitors which are known in the art and which may be utilized in the method for treating PF include acarbose, miglitol, voglibose, castanospermine, miglustat, and 1-deoxynojirimycin (DNJ). In some embodiments, a combination of GAA inhibitors may be administered to the subject.

In some embodiments, the method involves administering an effective amount of a GYS inhibitor, or a pharmaceutically acceptable salt thereof, to the subject. Certain GYS inhibitors which are known in the art and which may be utilized in the method for treating PF include those described in: U.S. Pat. No. 11,712,432 (the “'432 Patent”); Tang, B. et al., “Discovery and Development of Small-Molecule Inhibitors of Glycogen Synthase” Journal of medicinal chemistry 63, 3538-3551 (2020) (“Tang”); and Ullman, et al., “Small-Molecule Inhibition of Glycogen Synthase 1 for the Treatment of Pompe Disease and Other Glycogen Storage Disorders” Science Translational Medicine Vol. 16, Issue 730 (2024) (“Ullman”), the entire disclosure of each of which is incorporated herein in its entirety by reference. For example in some embodiments the GYS inhibitor can be: guaicaol (as disclosed in the '432 Patent);

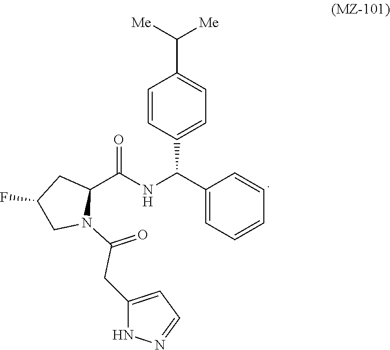 [SMILES: CC(C)c1ccc([C@@H](NC(=O)[C@@H]2C[C@@H](F)CN2C(=O)Cc2ccn[nH]2)c2ccccc2)cc1]

(as disclosed in Ullman); or a compound of the formula R

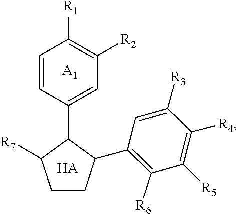

where HA is selected from

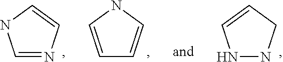

A₁ is selected from

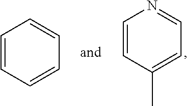

R₁ is selected from OH, H, CONH₂, F, Cl,

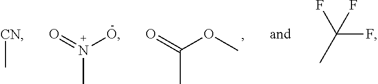

R₂ is selected from OCH₃, H, and Cl,
R₃ is selected from H and F,
R₄ is selected from H, OCH₃, and OH,
R₅ and R₆ are independently selected from H and OH, and
R₇ is selected from

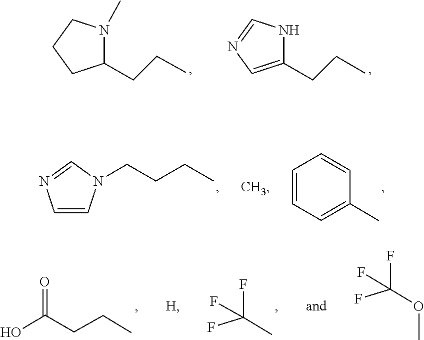

(as disclosed in Tang). In some embodiments, the GYS inhibitor can be

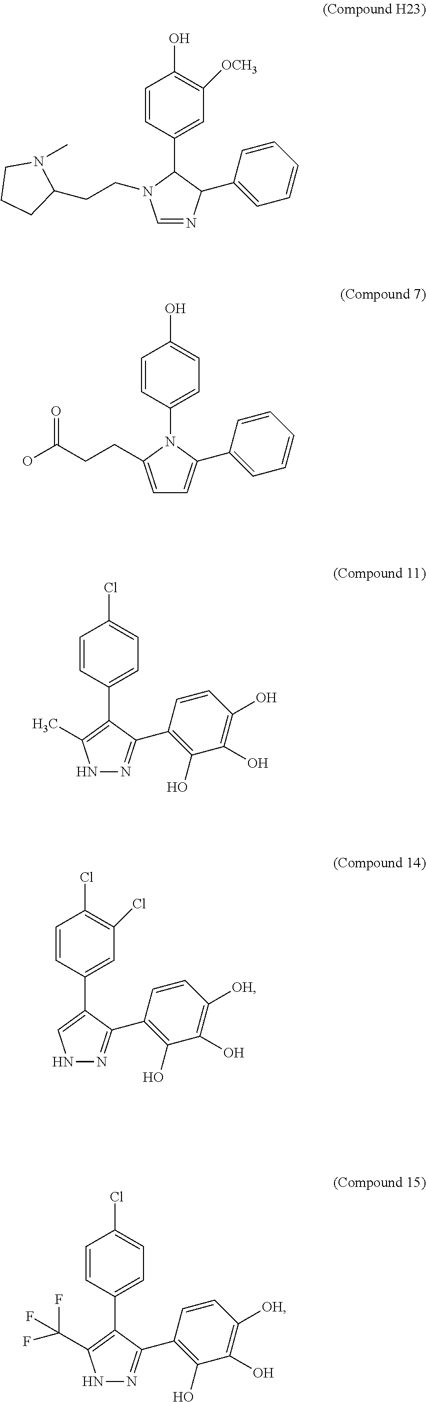 [SMILES: COc1cc(C2C(c3ccccc3)N=CN2CCC2CCCN2C)ccc1O ; O=C(O)CCc1ccc(-c2ccccc2)n1-c1ccc(O)cc1 ; Cc1[nH]nc(-c2ccc(O)c(O)c2O)c1-c1ccc(Cl)cc1 ; Oc1ccc(-c2n[nH]cc2-c2ccc(Cl)c(Cl)c2)c(O)c1O ; Oc1ccc(-c2n[nH]c(C(F)(F)F)c2-c2ccc(Cl)cc2)c(O)c1O]

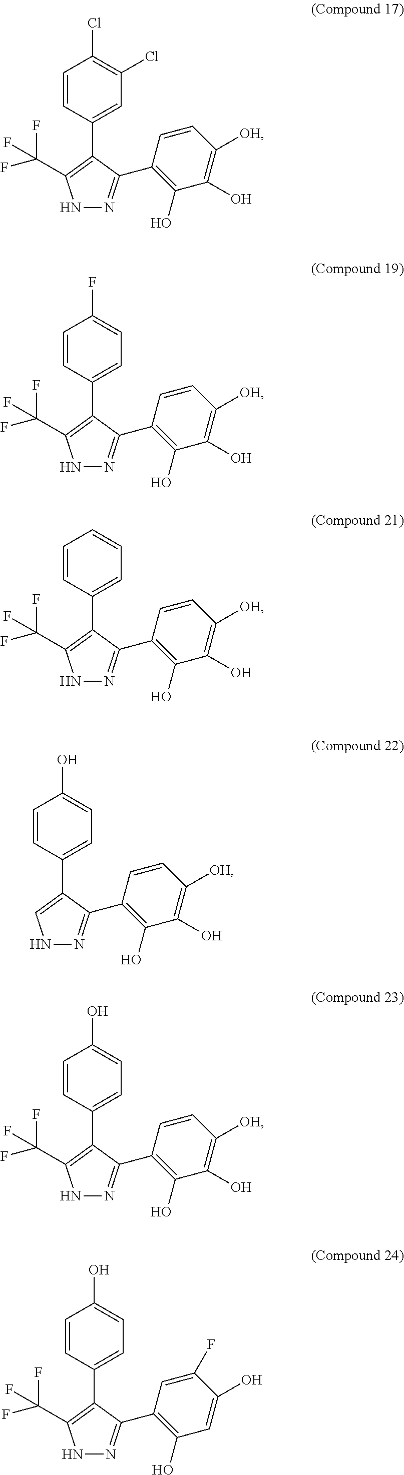 [SMILES: Oc1ccc(-c2n[nH]c(C(F)(F)F)c2-c2ccc(Cl)c(Cl)c2)c(O)c1O ; Oc1ccc(-c2n[nH]c(C(F)(F)F)c2-c2ccc(F)cc2)c(O)c1O ; Oc1ccc(-c2n[nH]c(C(F)(F)F)c2-c2ccccc2)c(O)c1O ; Oc1ccc(-c2c[nH]nc2-c2ccc(O)c(O)c2O)cc1 ; Oc1ccc(-c2c(-c3ccc(O)c(O)c3O)n[nH]c2C(F)(F)F)cc1 ; Oc1ccc(-c2c(-c3cc(F)c(O)cc3O)n[nH]c2C(F)(F)F)cc1]

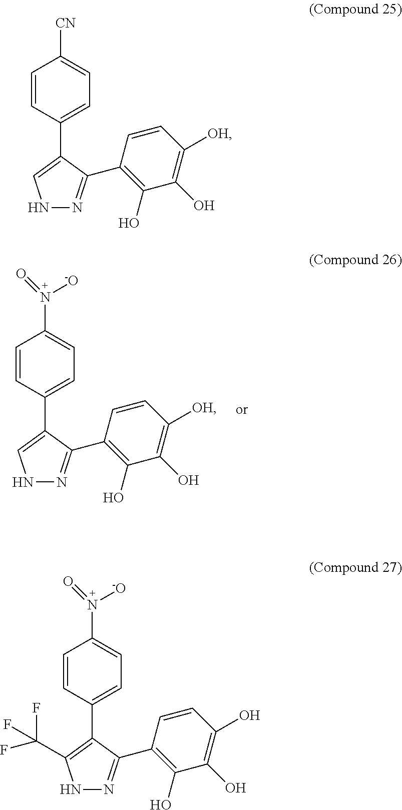

(as disclosed in Tang). In some embodiments, a combination of GYS inhibitors may be administered to the subject.

In some embodiments, the method involves administering an effective amount of a GP inhibitor, or a pharmaceutically acceptable salt thereof, to the subject. Certain GP inhibitors which are known in the art and which may be utilized in the method for treating PF include those described in: MedChem Express, CP-91149 Product Data Sheet, available at https://file.medchemexpress.com/batch_PDF/HY-13525/CP-91149-DataSheet-MedChemExpress.pdf (accessed Feb. 14, 2024) (“MedChem”); Huang, et al., “A Novel 5-Chloro-N-phenyl-1H-indole-2-carboxamide Derivative as Brain-Type Glycogen Phosphorylase Inhibitor: Validation of Target PYGB” Molecules 28(4): 1697 (2023) (“Huang”); and Bergans, et al., “Molecular Mode of Inhibition of Glycogenolysis in Rat Liver by the Dihydropyridine Derivative, BAY 3401: Inhibition and Inactivation of Glycgogen Phosphorylase by an Activated Metabolite” Diabetes 40(9): 1419-1426 (2000) (“Bergans”), the entire disclosure of each of which is incorporated herein by reference. For example, in some embodiments, the GP inhibitor can be

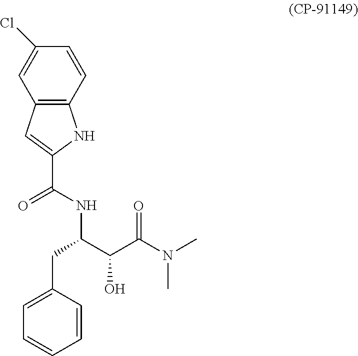 [SMILES: CN(C)C(=O)[C@H](O)[C@H](Cc1ccccc1)NC(=O)c1cc2cc(Cl)ccc2[nH]1]

(as disclosed in MedChem),

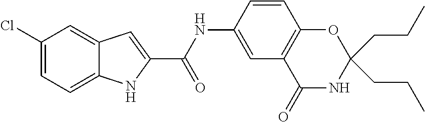 [SMILES: CCCC1(CCC)NC(=O)c2cc(NC(=O)c3cc4cc(Cl)ccc4[nH]3)ccc2O1]

(5-Chloro-N-phenyl-1H-indole-2-carboxamide derivative) (as disclosed in Huang), or

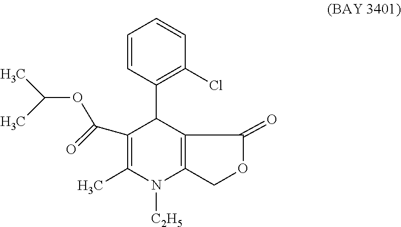 [SMILES: CCN1C(C)=C(C(=O)OC(C)C)C(c2ccccc2Cl)C2=C1COC2=O]

(as disclosed in Bergans). In some embodiments, a combination of GP inhibitors may be administered to the subject.

In some embodiments, the method may involve administering a combination including two or more of a glycogen phosphatase inhibitor, a GAA inhibitor, a GYS inhibitor, and a GP inhibitor, of pharmaceutically acceptable salts thereof, to the subject.

In some embodiments, the compound administered to the subject may comprise a derivative of one or more of the glycogen phosphatase, GAA, GYS, and GP compounds identified above.

In some embodiments, the method further involves identifying a subject as having PF or being at risk for PF. In some embodiments, the subject is identified as having PF. In some embodiments, the subject is identified as being at risk for PF. In some embodiments, the subject has a history of smoking and/or a family history of PF. In some embodiments, the subject has another risk factor associated with PF, such as prolonged exposure to air pollution.

A subject may be identified as having or being at risk of having PF based on one or more of: the subject's medical history; the subject's history of smoking, the subject's familial medial history, particularly with respect to familial history of PF; the results of one or more imaging tests, such as a chest x-ray, a computerized tomography (CT) scan, and an echocardiogram; the results of one or more lung function tests, such as pulmonary function testing, pulse oximetry, exercise stress test, arterial and blood gas test; the results of one or more tissue samples acquired, e.g., by bronchoscopy or surgical biopsy; and the results of one or more blood tests. In some embodiments, a subject may be identified as having or at risk of having PF based on a medical professional's diagnosis of the subject. As used herein, the phrase “at risk of having PF” is inclusive of instances in which a subject is suspected but not yet diagnosed as having PF as well as instances in which a subject is at risk of developing PF as a result of the subject's medical history, the subject's familial medical history, the subject having a history of smoking, the subject having a history of prolonged exposure to air pollution, and/or the subject having another risk factor associated with PF.

The presently-disclosed subject matter is further illustrated by the following specific but non-limiting examples. The following examples may include compilations of data that are representative of data gathered at various times during the course of development and experimentation related to the present invention.

### EXAMPLES

### Example 1: Spatial Metabolomics Reveals Glycogen as an Actionable Target for Pulmonary Fibrosis

MALDI mass spectrometry imaging (MSI) has greatly improved our understanding of spatial biology, however a robust bioinformatic pipeline for data analysis is lacking. Here, we demonstrate the application of high-dimensionality reduction and spatial clustering (HDR-SC) and histopathological annotation/prediction of MALDI-MSI datasets to assess tissue metabolic heterogeneity in human lung diseases. Using metabolic features identified from this pipeline, we hypothesized that metabolic channeling between glycogen and N-linked glycans is a critical metabolic process favoring pulmonary fibrosis progression. To test our hypothesis, we induced pulmonary fibrosis using bleomycin in two different mouse models with lysosomal glycogen utilization deficiency. Both mouse models displayed blunted N-linked glycan levels and nearly 90% reduction in endpoint fibrosis when compared to WT animals. Collectively, we provide conclusive evidence that lysosomal utilization of glycogen is required for pulmonary fibrosis progression. In summary, our study provides a roadmap to leverage spatial metabolomics to understand foundational biology in pulmonary diseases.

### Introduction

Mapping genotype to phenotype with near single-cell resolution and spatial definition is a major innovation in next-generation life science techniques. Recent improvements in RNA sequencing technologies that allow the detailed analysis of a single isolated cell from host tissues is a major advancement in achieving this goal. The resolution of single-cell RNA sequencing (scRNAseq) technology has impressive resolution that allows interrogation of tissue complexity and interactive networks at a cellular level¹⁻⁴. The scRNAseq workflow has allowed researchers to study complex cellular heterogeneity from diseased and healthy tissues, revealing complex and rare cell populations, while uncovering regulatory relationships and tracking distinct cell lineages during development⁵⁻⁸. Current single-cell approaches include cell-separation-based methods that are focused on molecular features such as proteins, lipids, and metabolites⁹⁻¹¹. To this end, in situ-based spatial technologies are beginning to emerge that can be mapped to anatomical regions, bridging the gap between genotype to phenotype analysis^{12,13}.

Complex carbohydrates such as N-linked glycans and glycogen are dynamic macrometabolites that impact complex and intertwined biochemical pathways^{14,15}. Anabolic pathways for the biosynthesis of glycogen and N-linked glycans span multiple cellular compartments including the nucleus, cytoplasm, endoplasmic reticulum, Golgi, and the plasma membrane¹⁴⁻¹⁷. Together, complex carbohydrates modulate a myriad of cellular functions including bioenergetics, epigenetics, protein-ligand binding, membrane transport activity, and protein turnover¹⁸. N-linked glycans are especially critical as structural components for anatomical regions, and are vital for immune modulation and organ function¹⁹. They are highly concentrated in pulmonary fibrosis and are part of the extracellular collagen, proteoglycan, and N-linked glycan matrix to support fibroblast growth and contribute to remodeled lung architecture, function and hindering gas exchange^{20,21}. Oligomerization of simple sugars to glycogen results in increased polarity and decreased solubility; therefore, quantitative, and spatial analyses of complex carbohydrates has been challenging. To address this limitation, a new technique was recently developed employing enzyme-assisted matrix-assisted laser desorption/ionization-mass spectrometry imaging (MALDI-MSI) that can simultaneously perform spatial profiling of both glycogen and N-linked glycans from formalin-fixed paraffin-embedded (FFPE) mammalian tissue sections in situ, preserving important anatomical regionsis^{15,22}.

MALDI-MSI utilizes robotic precision micromovement coupled to a Nd:YAG laser for spatial profiling of both organic and biological matter²³⁻²⁵. It is capable of high sampling aptitude with the ability to record tens of thousands of pixel-mapped spectra from a single tissue section coupled with spatial resolution²⁶⁻²⁹. The end result is a multidimensional, information-rich dataset containing metabolic features from normal and diseased tissues^{24,30-32}. Recent advances in MALDI-MSI enable the detection of complex carbohydrates such as proteoglycans³³, N-linked glycans³⁴, and glycogen architecture^{15,22} from FFPE tissue sections. The ability to utilize FFPE tissue sections dramatically increases the accessibility to banked clinical specimens with up to decades of patient-matched metadata. These analyses are hypothesis-generating in nature and can illuminate previously unknown regional or cellular-specific metabolic events that can be tested for translational therapeutic interventions.

In these studies, we demonstrate the application of high-dimensionality reduction and spatial clustering (HDR-SC) of MALDI-MSI datasets to map histopathological regions of human FFPE specimens. Further, we identified a set of unique carbohydrate features that predict fibrotic tissue with near 99.6% accuracy in a cohort of human diseased lung tissues. Targeted analysis of enriched features in fibrotic tissues reveal unique sets of complex carbohydrates as previously unidentified metabolic hallmark of human PF tissues. Finally, using both genetically modified mouse models and the bleomycin-induced mouse model of pulmonary fibrosis (PF), we demonstrate that glycogen utilization through the lysosomal salvage pathway and metabolic channeling between glycogen and N-linked glycans are critical for fibrosis development in vivo.

### Methods

Chemicals, Reagents, and Cell Lines. High-performance liquid chromatography-grade acetonitrile, ethanol, methanol, water, trifluoroacetic acid (TFA), bleomycin (B5507, Lot #SLCG3757), and recombinant isoamylase were purchased from Sigma-Aldrich. α-cyano-4-hydroxycinnamic acid (CHCA) matrix was purchased from Cayman Chemical. Histological-grade xylenes were purchased from Spectrum Chemical. Citraconic anhydride for antigen retrieval was obtained from Thermo Fisher Scientific. Recombinant PNGaseF Prime was obtained from N-Zyme Scientifics (Doylestown, PA, USA). Normal lung fibroblast isolated from adult lung tissue and diseased lung fibroblast isolated from adult idiopathic pulmonary fibrosis lung tissue were purchased from Lonza, USA (Cat #CC-2512 and CC-7231 respectively). ¹³C₆-glucose was purchased from Cambridge Isotope Laboratories, Inc (Cat #CLM-1396-pk).

Mouse Models. Mice were housed in a climate-controlled environment with a 14 (light)/10 (dark) hours light/dark cycle with temperature and humidity control. Water and solid diet provided ad libitum throughout the study. Epm2a^{-/-} mice, referred to as laforin knockout (LKO), were previously described⁹⁵. In brief, DNA sequence encoding the laforin dual specificity phosphatase domain was replaced with a neomycin domain. Wild-type (WT) C57BL/6J mice were purchased from Jackson Laboratory. Male and female mice between 3- and 6-months of age were anesthetized by i.p. injection of xylazine (AnaSed, Akorn) and ketamine (Ketathesia, Henry Schein) and received intratracheal (IT) instillation of a single dose of 0.3 U/kg of bleomycin sulfate from Streptomyces verticillus (Sigma-Aldrich) dissolved in 50 μL sterile saline. Control mice received saline IT. Mice were monitored and weighed daily throughout the study. At the experimental end point (24 hr after 21 days), mice were sacrificed followed by immediate resection of the lungs. The University of Kentucky Institutional Animal Care and Use Committee has approved all of the animal procedures.

Tissue Procurement. Mice were sedated with ketamine (100 mg/kg) and xylazine (10 mg/kg) followed by exsanguination and immediate resection of the lungs which were fixed in neutral-buffered formalin (NBF) for 24 hr then switched to 70% ethanol and paraffin-embedded for long-term storage. De-identified human patient tissues were obtained from the University of Kentucky Biospecimen Procurement and Translation Pathology Shared Resource Facility. Human tissues were also preserved in NBF and paraffin embedded for long-term storage. All hematoxylin & eosin (H&E) staining was performed by the University of Kentucky Biospecimen Procurement and Translation Pathology Shared Resource Facility using the method previously described⁹⁶.

Formalin-fixed Paraffin-Embedded Slide Preparation for MALDI-MSI. Tissues were sectioned at 4 μm and mounted on positively charged glass slides for MALDI imaging as previously described⁹⁷. Slides were heated at 60° C. for 1 hr. After cooling, tissue sections were deparaffinized by washing twice in xylene (3 min each). Tissue sections were then rehydrated by washing slides twice in 100% ethanol (1 min each), once in 95% ethanol (1 min), once in 70% ethanol (1 min), and twice in water (3 min each). Following washes, slides were transferred to a coplin jar containing citraconic anhydride buffer for antigen retrieval and the jar was placed in a vegetable steamer for 25 min. Citraconic anhydride buffer was prepared by adding 25 μL citraconic anhydride in 50 mL water and adjusted to pH 3.0 with HCl. After antigen retrieval, slides were dried in a vacuum desiccator prior to enzymatic digestion.

Glycogen and N-Glycan MALDI-Mass Spectrometry Imaging. An HTX spray station (HTX) was used to coat the slide with a 0.2 ml aqueous solution of isoamylase (3 units/slide) and PNGase F (20 mg total/slide). The spray nozzle was heated to 45° C. with a spray velocity of 900 m/min. Following enzyme application, slides were incubated at 37° C. for 2 hr in a humidified chamber, and dried in a vacuum desiccator prior to matrix application [α-cyano-4-hydroxycinnamic acid matrix (0.021 g CHCA in 3 ml 50% acetonitrile/50% water and 12 μL 25% TFA) applied with HTX sprayer]. For detection and separation of glycogen and N-glycans, a Waters SynaptG2-Si high-definition mass spectrometer equipped with traveling wave ion mobility was used. The laser was operating at 1000 Hz with an energy of 200 AU and spot size of 75 μm, mass range is set at 500-3000 m/z. Ion mobility setting were done according to previously established parameters^{17,96} with a trap entrance energy of 2V, trap bias of 85V, and DC/exist of 0V. Wave velocity settings were set to: trap 9.6 m/s, IMS 4.6 m/s, transfer 17.4 m/s. Wave height settings were set to: trap 4V, IMS, 42.7, transfer 4V, additional settings are variable wave ramp down of 1400 m/s. MALDI images were produced using the HDI software (Waters Corp) following built in peak integration function to account for mass drift over the MALDI run. All MALDI images were normalized to total ion current (TIC) within each pixel.

Quantitative Glycogen MALDI-Mass Spectrometry Imaging. 1, 10, 20, 40, 100, 1000 ng of purified rabbit liver glycogen were directly spotted on the microslide adjacent to the tissue section. An HTX spray station (HTX) was used to coat the slide with a 0.2 ml aqueous solution of isoamylase (3 units/slide). The spray nozzle was heated to 45° C. with a spray velocity of 900 m/min. Following enzyme application, slides were incubated at 37° C. for 2 hr in a humidified chamber, and dried in a vacuum desiccator prior to matrix application [α-cyano-4-hydroxycinnamic acid matrix (0.021 g CHCA in 3 ml 50% acetonitrile/50% water and 12 μL 25% TFA) applied with HTX sprayer]. For detection and separation of glycogen, Bruker timeTOF Flex mass spectrometer was used. The laser was operating at 10,000 Hz and 300 shots/second, mass range is set at 700-3000 m/z. MALDI images were produced using the SCILS software. Pixel information was exported using the SCILS API package in R studio and analysis was performed in prism. The molecular ion of 1175 m/z that corresponds to glucose polymer 6 was used to produce standard curve and absolute quantitation of glycogen levels in situ.

MALDI-MS Dataset Processing as Part of User Input Model. MALDI-MSI data files were processed to adjust for mass drift during the MALDI scan to enhance image quality and improve signal-to-noise ratio that will assist in the machine learning module. Raw MALDI-MSI data files were processed using an algorithm available within the HDI software (Waters Corp). To adjust for mass drift during the MALDI scan, raw files were processed using a carefully curated and established list of 50 MALDI matrix peaks (m/z) and 155 glycogen and N-linked glycan peaks (m/z) listed in Tables 3A and 3B. Files were processed at a sample duration of 10 sec at a frequency rate of 0.5 min, and an m/z window of <1 Da, using an internal lock mass of previously defined N-linked glycan 1257.4296 m/z with a tolerance of lamu and a minimum signal intensity of 100,000 counts. Three to five glycans were spot checked for abundance and known spatial distribution as a positive control⁹⁷. Post-processed MALDI data files were exported in a tabular format as .CSV for input into the machine learning module.

Data Availability. The MALDI imaging data generated in this study have been deposited in the DRYAD database under https://doi.org/10.5061/dryad.jwstgjqfg. The pool metabolomics data used in this study are available in the Metabolomics Workbench database under accession code/study ID ST002547 [https://doi.org/10.21228/M8QFOW].

Dimensionality Reduction and Clustering as Part of Computer-Based Module. Normalization, high dimensional clustering, and UMAP and spatial plots, were done using the Python® software environment⁹⁸. First, each complex carbohydrate feature was normalized on total ion count (TIC) within individual pixels. The Leiden algorithm³⁶ automatically performed spatially agnostic clustering of pixels and optimized cluster approximation based on similarities in their carbohydrate features. Uniform Manifold Approximation and Projection (UMAP)³⁷ was applied to embed high dimensional carbohydrate features of pixels in a low dimensional space for data visualization and interpretation. The clustering results were visualized by a pixel-colored UMAP plot in 2D feature space and a pixel-colored spatial plot mapping pixel to the original X-Y coordinates recorded by MALDI-MSI. Both plots are qualified for revealing tissue anatomy, histopathology, and heterogeneity. Leiden clustering algorithm and UMAP plot generation are built-in modules as part of the Scanpy package available within Python®⁹⁹. Code is available for free at github.com/maldiUKY/HDC-SC. Codes are tested and functional under the MacBook Pro IOS environment.

Discovery of Overrepresented Complex Carbohydrate Features and Pathway Enrichment. Multivariate analyses of all complex carbohydrate features within unique clusters were analyzed using the Metaboanalyst 5.0 software (n=3 technical regions of interest [ROIs] per cluster or biological sample) as previously described¹⁰⁰. Log transformation and auto scaling were used for normalization. Heatmaps were generated with the top 50 ranked features and hierarchical clustering was performed using the Euclidean distance measure and Ward linkage. Pathway enrichment analysis based on overrepresented molecular features were done manually based on previously established pathways of N-linked glycosylation and glycogen metabolism¹⁰¹. All MALDI-MSI images of glycogen and N-glycans were generated using the HDI software (Waters Corporation). Representative glycan structures were generated in GlycoWorkbench.

Histopathology Assessments. Histopathology assessments were performed by four board certified clinical pathologists independently to improve rigor and reproducibility. All H&E slides were assessed in a CLIA certified pathology laboratory using an inverted brightfield microscope (Olympus BX43). Image analysis was performed using the HALO image analysis software (Indica labs). Assessments were made based on the spatial clustering produced by HDR-SC and histopathology assessment from an immediate adjacent section 4 μm apart.

Immunohistochemistry. Fixed lung tissues were sectioned at 10 μm and immunohistochemistry was performed at the Biospecimen Procurement and Translation Pathology Shared Resource Facility using the method previously described¹⁷. Briefly, tissue was rinsed with 0.01M PBS, incubated for 1 hour in 5% normal goat serum in 0.3% Triton X-100 in PBS. The tissue was then incubated at room temperature in the primary antibody (below) and diluted in 1% normal goat serum, followed by 3×15 minute rinses in 0.05% Tween-20 in PBS. Tissue was then incubated for 1 hour in secondary antibody diluted in 1% normal goat serum in PBS. Antibodies used for other markers are glycogen synthase (LSBio, Cat #LS-B12901), glycogen phosphorylase brain isoform (LSBio, Cat #LS-B4749), and glycogen phosphorylase muscle isoform (Protech, 1Cat #9716-AP). Digital images were acquired through the ZEISS Axio Scan.Z1 high resolution slide scanner. Quantitative image analysis was performed using the Halo software (Indica labs) using the multiplex IHC modules.

Co-Immunofluorescence. Fixed brains were embedded in paraffin and sectioned at 4 μm onto slides by the University of Kentucky Biospecimen Procurement and Translation Pathology Shared Resource Facility. Slides were dewaxed, rehydrated, and heated to 60° C. for 30 minutes in citraconic anhydride buffer (pH 6.5) for antigen retrieval. After cooling, slides were incubated in primary antibody (Table 1A) followed by incubation with a secondary antibody (Table 1B). Slides were then cover slipped using Southern Biotech DAPI Fluoromount-G (Cat. 591 #0100-20). Digital images were acquired as 12 Z-stacks through the Zeiss Axio Scan Z.7 digital slide scanner at 40× magnification and processed using HALO software (v3.3.2541.345, Indica Labs, Albuquerque, NM).

TABLE 1A

| Primary | | Company and | | |
|---|---|---|---|---|
| antibodies | Host | catalog # | Dilution | Conditions |
| Glycogen | Mouse | IV58b6 | 1:500 | 1 hour, RT |
| GAA | Rabbit | Proteintech | 1:100 | 1 hour, RT |
| | | 14267-1-AP | | |
| LAMP2 | Rabbit | Abcam ab 125068 | 1:100 | Overnight, |
| | | | | RT |
| Alpha smooth | Rabbit | GeneTex | 1:100 | Overnight, |
| muscle actin | | GTX100034 | | RT |
| DAPI | | Novus NBP2-31156 | 1:5000 | 5 minutes, |
| | | | | RT |

TABLE 1B

| Secondary | | Company and | | |
|---|---|---|---|---|
| antibodies | Fluorophore | catalog # | Dilution | Conditions |
| Goat anti-rabbit | Alexa 488 | A-11034 | 1:400 | 1 hour, RT |
| Goat anti-mouse | Alexa 647 | A-21236 | 1:400 | 1 hour, RT |

Lung Fibrosis Assessments. Lung fibrosis was evaluated by Ashcroft score using H&E sections. For analysis, two images from four micrographs of whole lung sections were randomly selected from four mice. These randomly selected images were individually assessed in a blinded manner with an index of 0-8: 0, normal lung; 1, minimal fibrous thickening of alveolar or bronchiolar walls; 2-3, moderate thickening of walls without obvious damage to lung architecture; 4-5, increased fibrosis with definite damage to lung structure and formation of fibrous bands or small fibrous masses; 6-7, severe distortion of structure and large fibrous areas, evidence of “honeycomb lung”; 8, total fibrous obliteration of the field. Soluble collagen content from bronchoalveolar lavage fluid (BALF) was determined using the Sircol assay (Biocolor), according to the manufacturer's instructions. Sircol dye bound to collagen was evaluated by a microplate reader at 555 nm. BALF was collected by performing two washes of 0.7 mL with PBS+EDTA (0.1 mM) on ice.

Collagen Targeted Imaging Proteomics. Samples were prepared as previously described for targeted collagen and extracellular matrix peptide imagingo¹⁰²⁻¹⁰⁶. Briefly, deglycosylated samples^{107,108} were antigen retrieved with 10 mM Tris, pH 9, autosprayed (M5, HTX-Technologies) with 0.1 tg/tL collagenase type III (Worthington) dissolved in ammonium bicarbonate pH 7.4, 1 mM CaCl₂), and incubated at 38.5° C. with ≥85% humidity for 5 hours. The matrix α-Cyano-4-hydroxycinnamic acid (CHCA, Sigma Aldrich) was dissolved in 1.0% trifluoracetic acid (Sigma), 50% acetonitrile (LC-MS grade, Fisher Chemical) and autosprayed (M5, HTX Technologies) onto tissue. Tissues were imaged on a MALDI-QTOF (timsTOF-flex, Bruker) in positive ion mode over m/z range 7002500. Laser was adjusted to 20 tm² and each pixel consisted of 300 laser shots. Images were collected with a laser step size of 60 tm. Data was visualized in SCiLS Lab Software (v2022b, Bruker) and processed for image segmentation and principal component analysis. Peak data were exported by mean spectrum processed as peak maximum and further statistical comparisons were done using Metaboanalyst 5.0 and GraphPad Prism 9.0. Exported peak intensities were visualized as heatmaps using the TM4 MultiExperiment Viewer suite¹⁰⁹ with clustering by Manhattan metric and single linkage.

Cell Culture and ¹³C-Glycogen Tracing in Fibroblast Cell Lines. Normal and diseased lung fibroblast were maintained in fibroblast growth medium (Lonza, Cat #CC-3132) supplement with the FGM-2 SingleQuots supplement (Lonza, Cat #CC-4126) in 10 cm dishes. For the ¹³C-glycogen enrichment experiment, fibroblast cells were allowed to reach ˜50% confluency, followed by the addition of DMEM base media supplemented with 10 mM ¹³C₆-glucose, 2 mM Gln, 10% dialyzed fetal bovine serum in a CO2 incubator maintained at 37° C. At the designated timepoints, cells were washed with cold PBS three times followed by extraction with 50% methanol and separated into polar, insoluble fraction that contains the glycogen. For the ¹³C-glycogen washout experiment, fibroblast cells were grown in ¹³C₆-glucose enrichment media for 48 hours followed by the replacement of DMEM base media supplement with ¹²C-glucose. At designated timepoints, cells were washed with cold PBS three times followed by extraction with 50% methanol and separated into polar and the glycogen-containing insoluble fraction.

Gas-Chromatography-Mass Sepctrometry (GCMS) Analysis of ¹³C-Enriched Glycogen. GCMS assessment of glycogen was done using method previously described^{58,59}. Briefly, insoluble fraction was subject to acid hydrolysis with 2N HCL for 3 hours at 98° C. The reaction was quenched with 2N NaOH for subsequent experiments. For GC-MS analysis, samples were first dried in a SpeedVac (Thermo), followed by sequential addition of 20 mg/ml methoxylamine hydrochloride in pyridine, and then the trimethylsilylating agent N-methyl-N-trimethylsilyl-trifluoroacetamide (MSTFA) was added followed by GCMS analysis. GC-MS protocols were similar to those described previously^{58,59}. The electron ionization (EI) energy was set to 70 eV. Scan (m/z: 50-800) and selected ion monitoring mode were used for qualitative measurement and isotope monitoring, respectively. Ions used for the enrichment of glycogen-derived glucose are 319, (unlabeled) and 320, 321, 322, 323, (labeled). Batch data processing and natural ₁₃C labeling correction were performed using the Data Extraction for Stable Isotope-labeled metabolites (DExSI) software package.

Targeted Analysis of Metabolites by Liquid-Chromatography-Mass Spectrometry. Approximately 20 mg of pulverized, frozen tissue was extracted in 3 ml ice-cold 60% acetonitrile by extensively vortexing. Next, 1 ml chloroform was added to each sample and samples were mixed by manual shaking followed by centrifuging at 3,200 g for 20 minutes at 4° C. The aqueous layer was moved to a new tube and lyophilized. The middle layer containing protein and insoluble material was transferred to a new tube, washed twice with 50% methanol, once with 100% methanol, and briefly dried in a speed-vac. After drying, the insoluble material was hydrolyzed by heating samples in 3N HCl at 95° C. for 2 hours. 100% methanol was added to hydrolysate to achieve a final concentration of 50% methanol, samples mixed, centrifuged at 18,000 g for 10 min at 4° C., then supernatant dried on a speed vac. Polar metabolites were separated by HILIC chromatography using an Agilent InfinityLab Poroshell 120 HILIC-Z column (2.7 m, 2.1×150 mm) with a binary solvent system of 10 mM ammonium acetate in water, pH 9.8 (solvent A) and ACN (solvent B) with a constant flow rate of 0.25 ml/min. The column was equilibrated with 90% solvent B. Polar metabolites were resuspended in a 1:1 mixture of solvents A:B, centrifuged at 18,000 g for 5 minutes at 4° C., then transferred to glass vials. 4 μl of each sample was injected and the gradient proceeded from 0-15 min linear ramp 90% B to 30% B, 15-18 min isocratic flow of 30% B, 18-19 min linear ramp from 30% B to 90% B, and 19-27 min column regeneration with isocratic flow of 90% B. Metabolites were measured using an Agilent 6545 quadrapole-time of flight mass spectrometer (MS) coupled to an Agilent 1290 Infinity II UHPLC. Individual samples and standards were acquired with full scan MS in negative mode. Peaks for the deprotonated [M-H]⁻ ions were extracted and integrated using Agilent Qualitative Analysis software (retention time, formula, and m/z are presented in Table 2). Polar metabolite peaks were normalized to the amino acid content in the hydrolyzed insoluble biomass fraction. Samples were resuspended in 70 μl of 20 mg/ml methoxyamine in pyridine and incubated for 1.5 h at 37° C. Samples were centrifuged at 18,000 g for 5 minutes and 50 μl of each sample was transferred to a glass vial. 80 ul of N-Methyl-N-(trimethylsilyl)trifluoroacetamide+1% chlorotrimethylsilane (Themo Scientific) was added to each sample and incubated at 37° C. for 30 minutes. Samples were then analyzed by gas chromatography mass spectrometry (GCMS) for as described above.

TABLE 2
Annotated metabolites with their molecular
formula, adduct, m/z, and retention time.

| | | | | Retention |
|---|---|---|---|---|
| Molecule | Formula | Adduct | m/z | Time (min) |
| UDP-glucose | C15H24N2O17P2 | [M − H]− | 565.0477 | 5.612 |
| Malate | C4H6O5 | [M − H]− | 133.0142 | 5.546 |
| Glutamic Acid | C5H9NO4 | [M − H]− | 146.0459 | 5.745 |
| Alanine | C3H7N1O2 | [M − H]− | 88.0404 | 5.387 |
| Glutamine | C5H10N2O3 | [M − H]− | 145.0619 | 5.513 |
| Citrate | C6H8O7 | [M − H]− | 191.0197 | 4.601 |
| Glucose | C6H13O9P | [M − H]− | 259.0224 | 6.1 |
| 6-phosphate | | | | |
| Fructose | C6H13O9P | [M − H]− | 259.0224 | 6.45 |
| 6-phosphate | | | | |

Statistical Analysis. All dimensionality reduction and clustering analyses were performed using the Python® software environment. Both clustering and differential analyses were conducted with the Scanpy package by Leiden algorithm and Wilcoxon Rank-Sum test, respectively. For Metaboanalyst multivariate analyses of all glycans, log transformation and auto scaling were used for normalization. Heatmaps were generated based on the Euclidean distance measure and the Ward clustering algorithm. Glycans with variable importance in projection (VIP) scores >1.5 based on partial least squares discriminant analysis (PLS-DA) were selected for further analysis. For biomarker analysis, areas under the curve (AUC) were obtained using multivariate receiver operating characteristic (ROC) analysis based on the linear SVM classification method and SVM feature ranking method. Statistical analysis for discovery of overrepresented complex carbohydrate features in patient samples and in vivo studies were carried out using GraphPad Prism 9.0. All numerical data are represented as mean±S.E.M Column analysis was performed using one-way ANOVA or t-test. A P value less than 0.05 was considered statistically significant. The statistical parameters for each experiment can be found in the figures and figure legends.

### Results

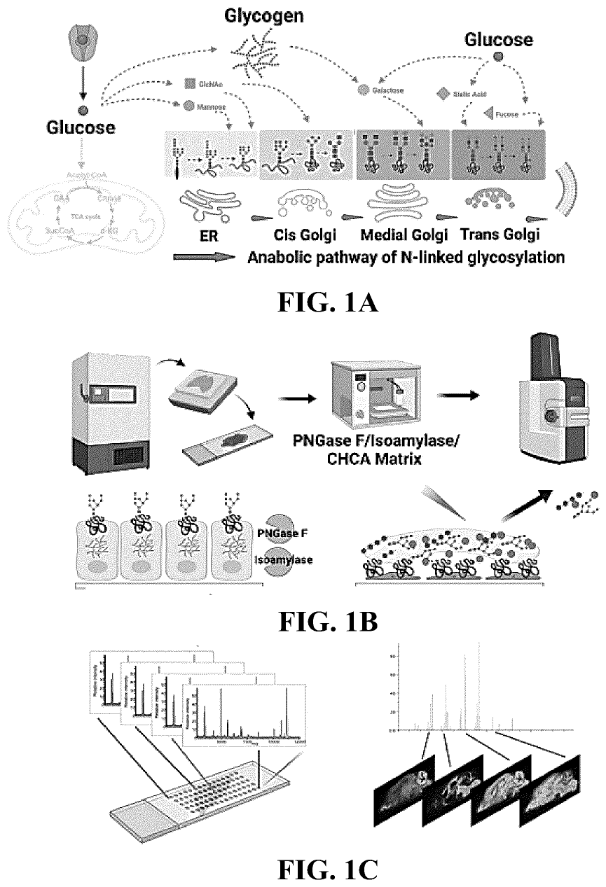

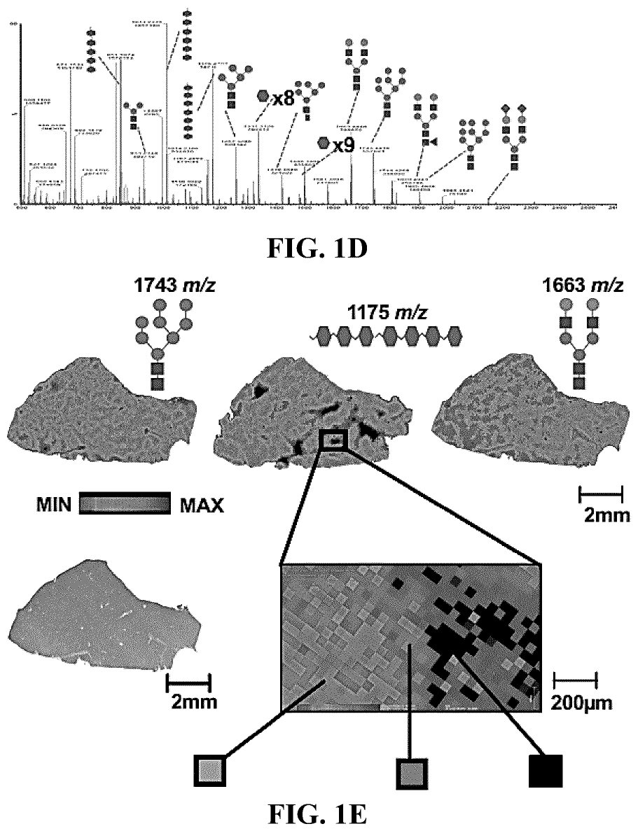

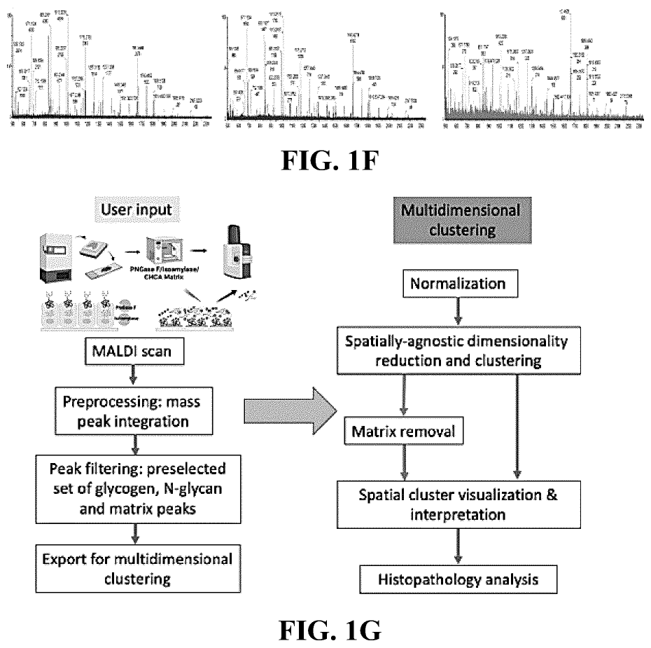

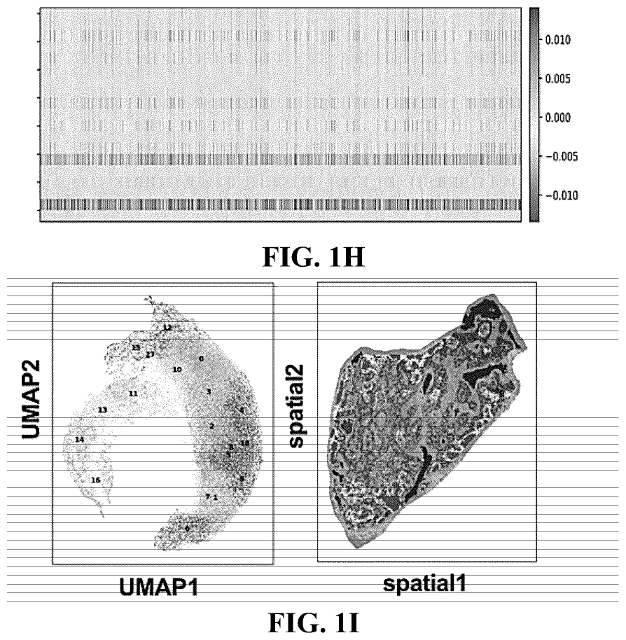

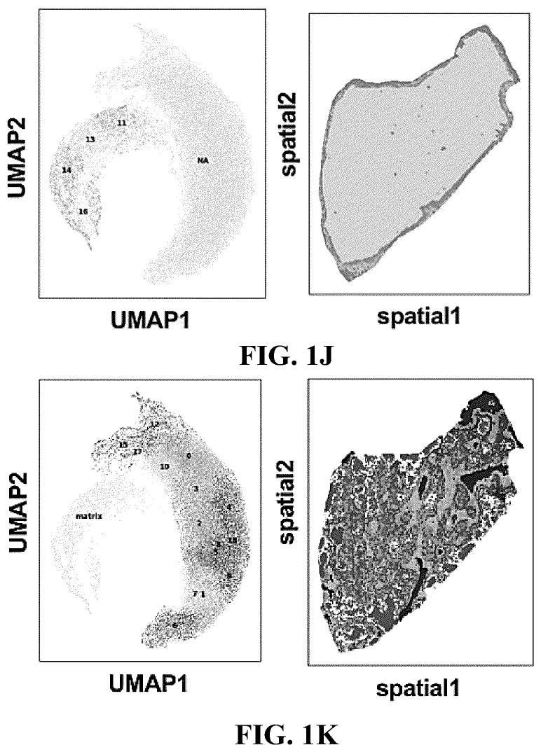

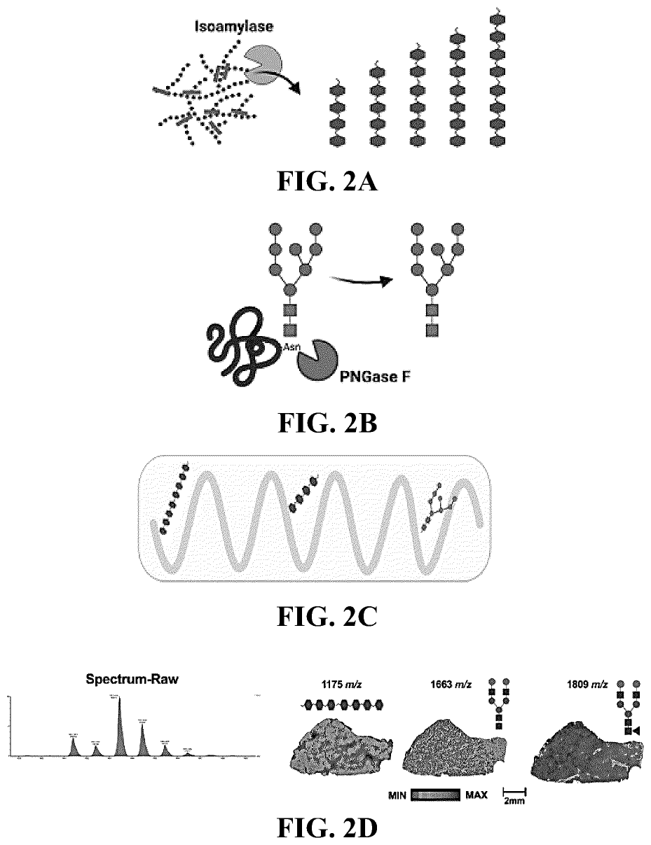

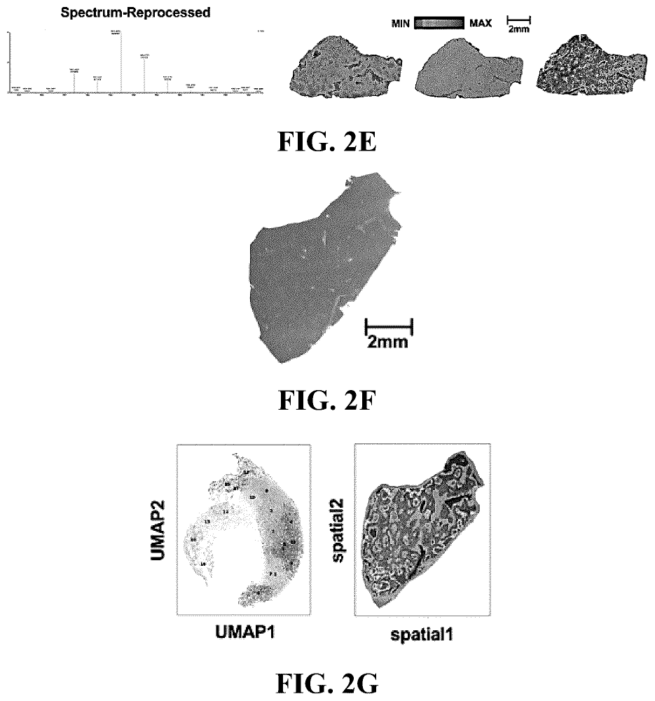

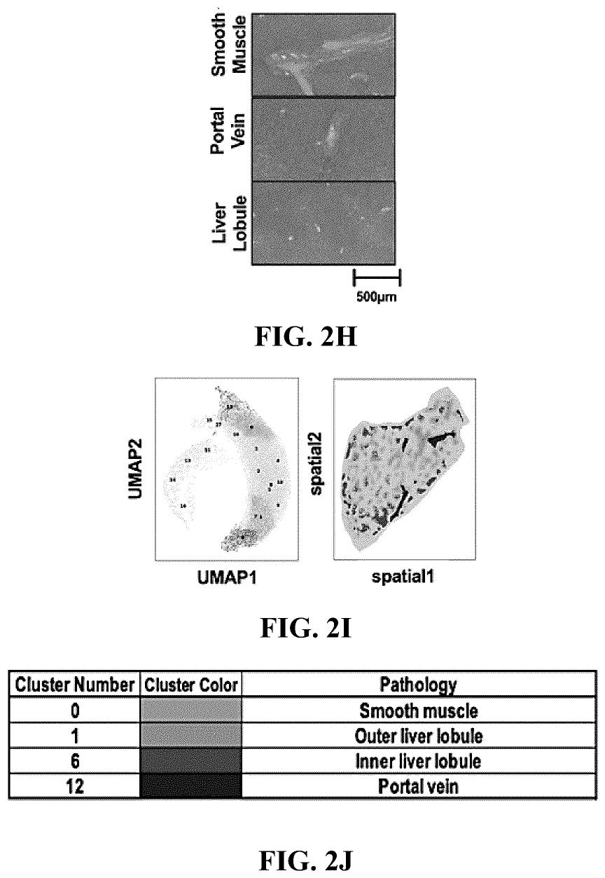

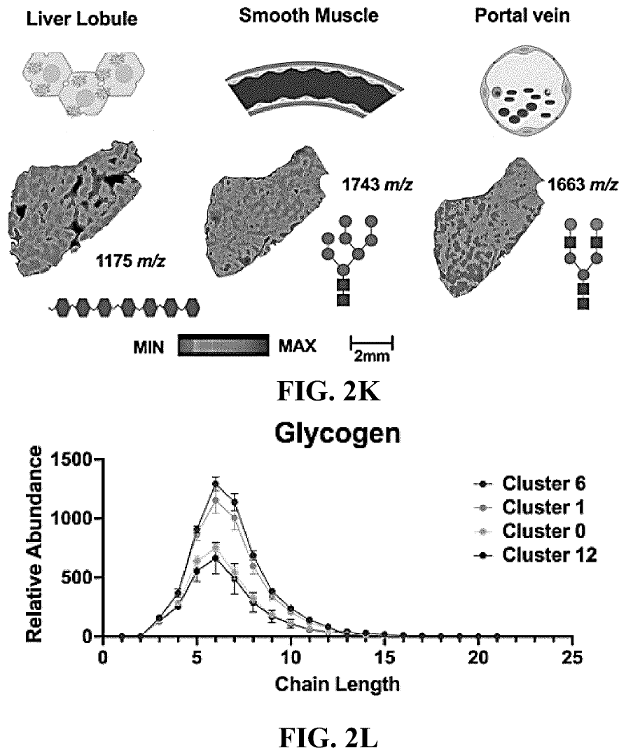

HDR-SC of MALDI-MSI Dataset Reveal Tissue Anatomical Regions. Anabolic pathways for complex carbohydrates such as glycogen and N-linked glycans are critical facets of glucose metabolism, and emerging work demonstrates that they are metabolically channeled through common substrates^{14,15}. Comprehensive profiling of both can elucidate compartmentalized glucose metabolism within a cell (FIG. 1A). Recently, a technique was developed for the multiplexed imaging of N-linked glycans and glycogen using MALDI-MSI (FIG. 1B)^{15,35}. This method takes advantage of two highly specific enzymes: isoamylase, which cleaves glycogen to release linear glucose chains, and peptide: N-glycosidase F (PNGaseF), which liberates N-linked glycans from proteins (FIGS. 2A-2B). Subsequently, linear glucose chains and N-linked glycans can be distinguished by ion mobility separation during MALDI-MSI (FIG. 2C)¹⁵.

The multidimensional nature of MALDI-MSI datasets is highlighted by the fact that each pixel from a MALDI-MSI experiment contains a full ion spectrum of detectable molecular features (FIG. 1C-1F). To further explore the full potential of the multiplexed MALDI-MSI workflow, we tested whether HDR-SC of MALDI-MSI datasets can match spatial tissue anatomy using MALDI-MSI of a human liver section (FIG. 1G). First, a carefully curated and established list of 50 MALDI matrix peaks (m/z) and 155 glycogen and N-linked glycan peaks (m/z) were selected for HDR-SC analysis (Tables 3A and 3B), then we performed peak integration to account for mass drift during the MALDI scan and improve image resolution (FIGS. 2D-2E). Normalized input data (FIG. 1H) undergoes dimensionality reduction and clustering using the Leiden clustering algorithm³⁶, and the results are presented as a spatial plot and a Uniform Manifold Approximation and Projection (UMAP) plot (FIG. 1I)³⁷. UMAP visualization can accurately identify α-cyano-4-hydroxycinnamic acid (CHCA) matrix clusters (often presented as a standalone cluster on the UMAP plot) and can be omitted from further analyses (FIG. 1J). Spatial extrapolation of remaining clusters based on pixel coordinates revealed unique spatial regions within the liver section (FIG. 1K).

TABLE 3A
Molecular structures of N-linked glycans and their
corresponding monoisotopic mass (m/z) rounded to
the nearest whole number.

| m/z | Representative Struture |
|---|---|
| 771 | 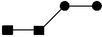 |
| 933 | 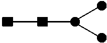 |
| 1079 | 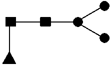 |
| 1095 | 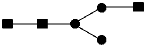 |
| 1136 | 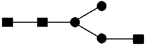 |
| 1257 | 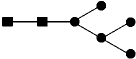 |
| 1282 | 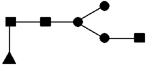 |
| 1298 | 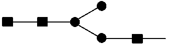 |
| 1339 | 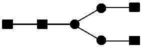 |
| 1419 | 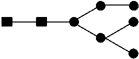 |
| 1444 | 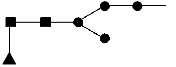 |
| 1460 | 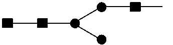 |
| 1485 | 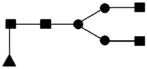 |
| 1501 | 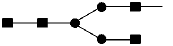 |
| 1542 | 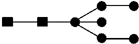 |
| 1581 | 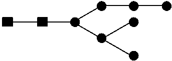 |
| 1589 | 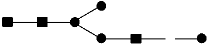 |
| 1606 | 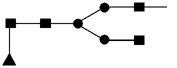 |
| 1622 | 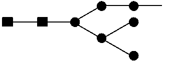 |
| 1647 | 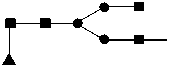 |
| 1663 | 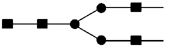 |
| 1688 | 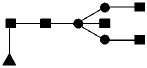 |
| 1743 | 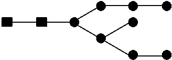 |
| 1791 | 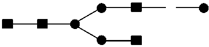 |
| 1793 | 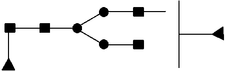 |
| 1809 | 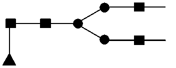 |
| 1825 | 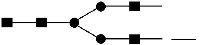 |
| 1850 | 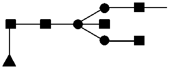 |
| 1866 | 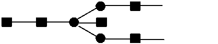 |
| 1891 | 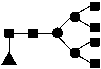 |
| 1905 | 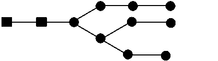 |
| 1911 | 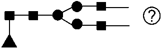 |
| 1954 | 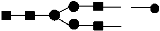 |
| 1976 | 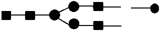 |
| 19 [symbol] 6 | 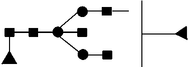 |
| 2012 | 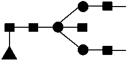 |
| 2026 | 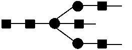 |
| 2053 | 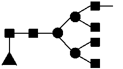 |
| 2057 | 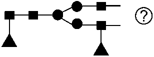 |
| 2100 | 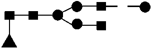 |
| 2122 | 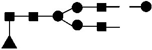 |
| 2141 | 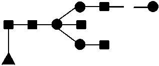 |
| 2157 | 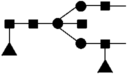 |
| 2174 | 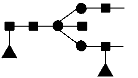 |
| 2215 | 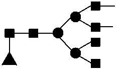 |
| 2231 | 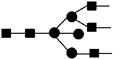 |
| 2245 | 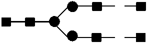 |
| 2267 | 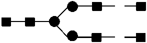 |
| 2304 | 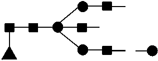 |
| 2319 | 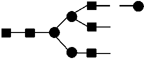 |
| 2320 | 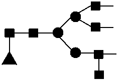 |
| 2341 | 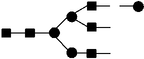 |
| 2360 | 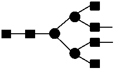 |
| 2377 | 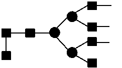 |
| 2393 | 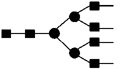 |
| 2448 | 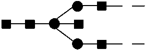 |
| 2465 | 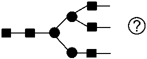 |
| 2522 | 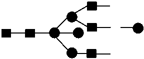 |
| 2539 | 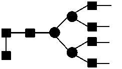 |
| 2610 | 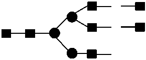 |
| 2653 | 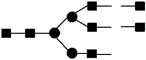 |
| 2684 | 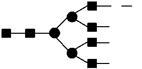 |
| 2685 | 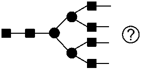 |
| 2779 | 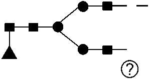 |
| 2815 | 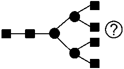 |
| 2832 | 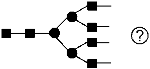 |
| 2905 | 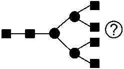 |
| 2978 | 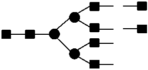 |

[symbol]

indicates data missing or illegible when filed

TABLE 3B

| Molecular structure of representative linear oligosaccharide chains and | | | | | | |
|---|---|---|---|---|---|---|
| phospho-linear oligosaccharide chains and their corresponding | | | | | | |
| monoisotopic mass (m/z) rounded to the nearest whole number. | | | | | | |
| m/z | 527 | 689 | 851 | 1013 | 1175 | 1337 |
| Compo- sition | 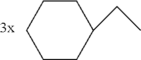 | 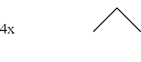 | 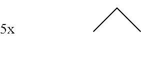 | 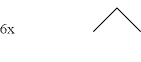 | 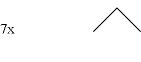 | 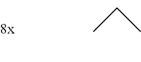 |
| m/z | 1499 | 1662 | 1824 | 1986 | 214 [symbol] | 2310 |
| Compo- sition | 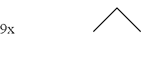 | 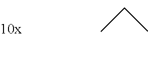 | 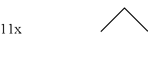 | 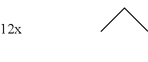 | 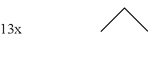 | 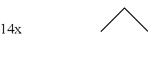 |
| m/z | 2472 | 2634 | 2797 | 3121 | 3443 | |
| Compo- sition | 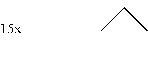 | 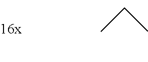 | 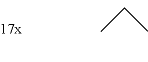 | 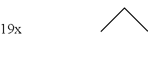 | 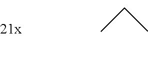 | |

| m/z | 569 | 731 | [symbol] 93 | 1056 |
|---|---|---|---|---|
| Compo- sition | 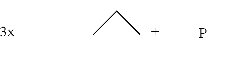 | 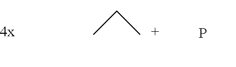 | 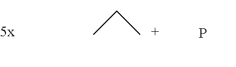 | 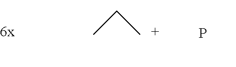 |
| m/z | 1218 | 337 [symbol] | 19 [symbol] [symbol] | 1704 |
| Compo- sition | 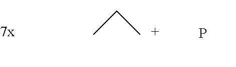 | 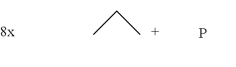 | 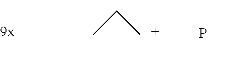 | 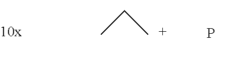 |
| m/z | 18 [symbol] [symbol] | 202 [symbol] | 2189 | 2351 |
| Compo- sition | 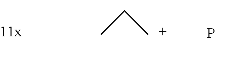 | 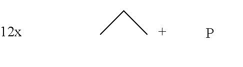 | 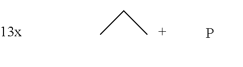 | 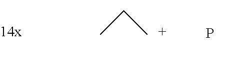 |
| m/z | 2514 | 2 [symbol] 7 [symbol] | 2 [symbol] 3 [symbol] | 3000 |
| Compo- sition | 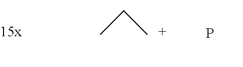 | 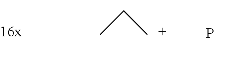 | 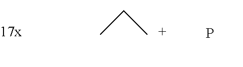 | 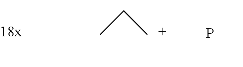 |

| | | |
|---|---|---|
| m/z | 3162 | 3324 |
| Compo- sition | 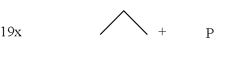 | 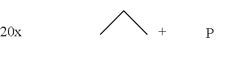 |

[symbol]

indicates data missing or illegible when filed

To test whether unique pixel clusters match known liver anatomical regions and structures, we performed detailed histology analysis using a liver section stained with hematoxylin and eosin (H&E) followed by annotation by expert clinical surgical pathologists (FIGS. 2F-2G). By matching to histology evaluation in a blinded fashion, clustering results matched accurately with histological features such as liver lobules, smooth muscles, and portal veins (FIGS. 211-2I). Interestingly, unique clusters were observed for inner and outer layers within the liver lobules (cluster 6 and 1, respectively) and separated both longitudinal and crosscut sections of the portal vein surrounded by smooth muscle (cluster 12 and 0, respectively) (FIG. 2J). Glycogen features are predominately enriched in the liver lobule clusters while smooth muscle and portal veins are enriched for N-linked glycans (FIG. 2K). Finally, we performed glycogen structural analyses between inner and outer liver lobules and portal veins. As expected, the hepatocytes within liver lobules have both shorter and longer chain length distribution than at the region near the portal vein. The hepatocytes within the central portion of the lobule have higher short glycogen chain abundance than the outer layer; however, there were no differences between longitudinal and cross-section portal vein regions (FIG. 2L). These findings demonstrate the ability of HDR-SC of MALDI-MSI datasets to accurately identify tissue microanatomy.

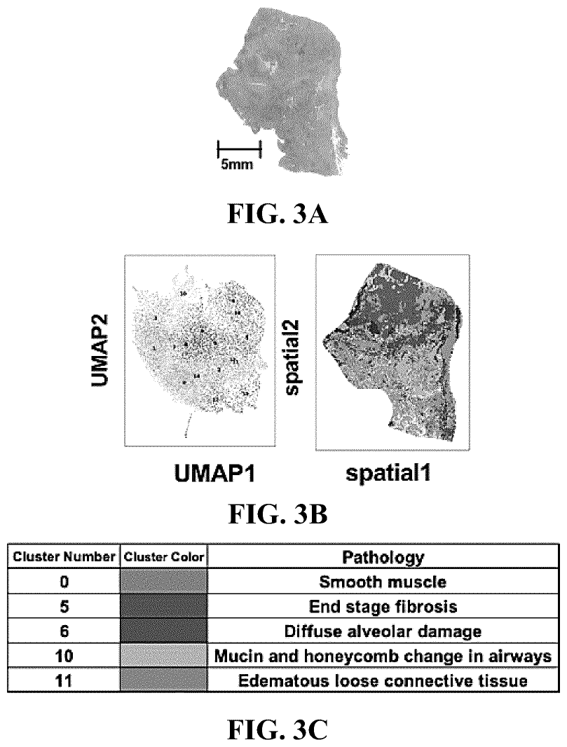

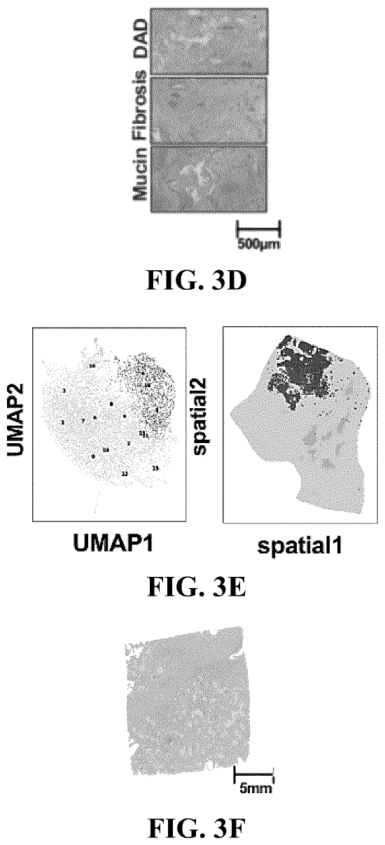

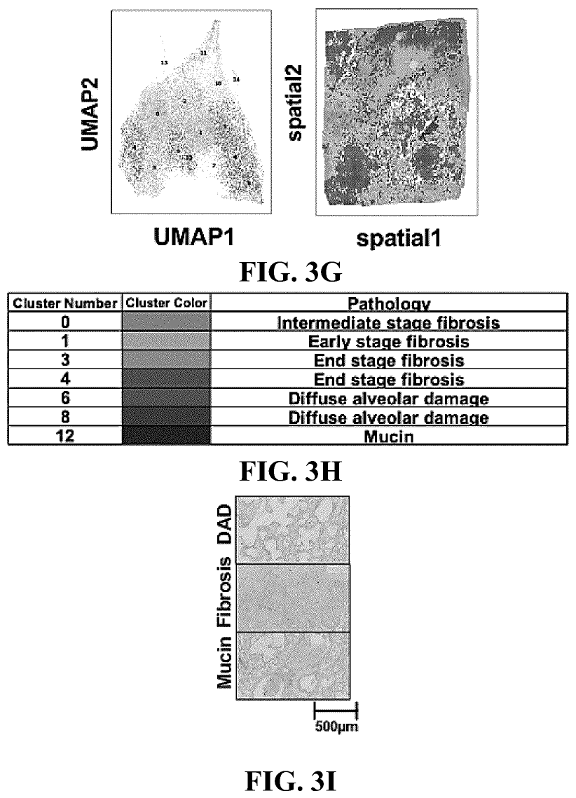

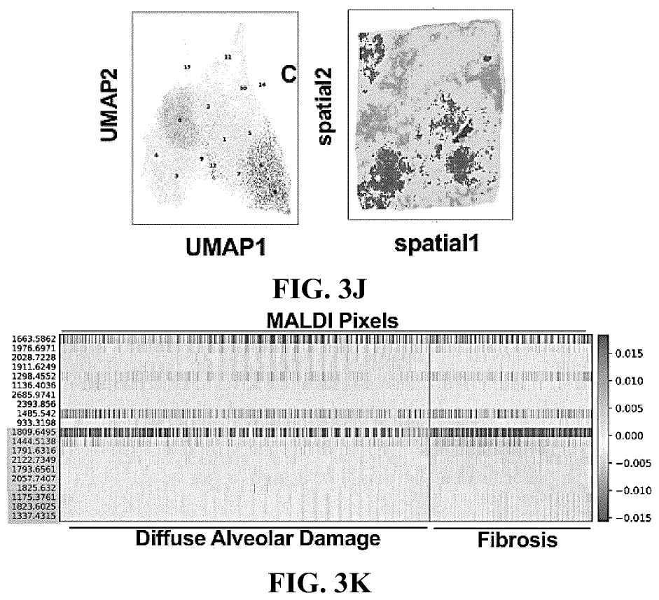

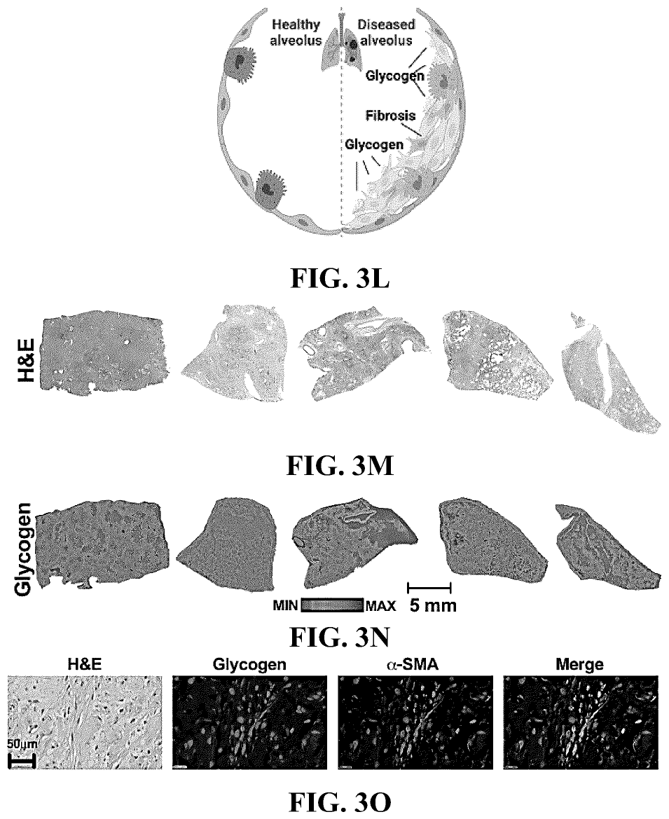

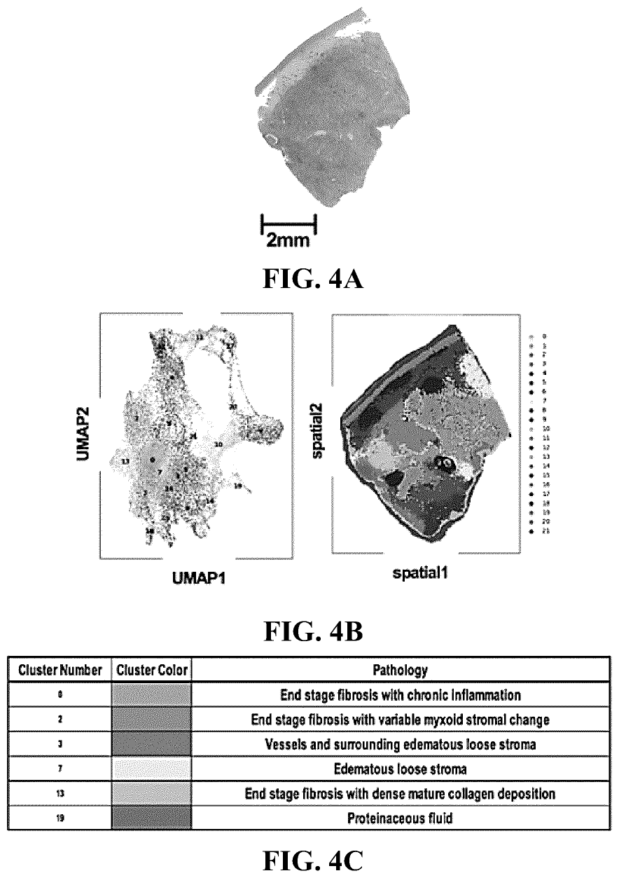

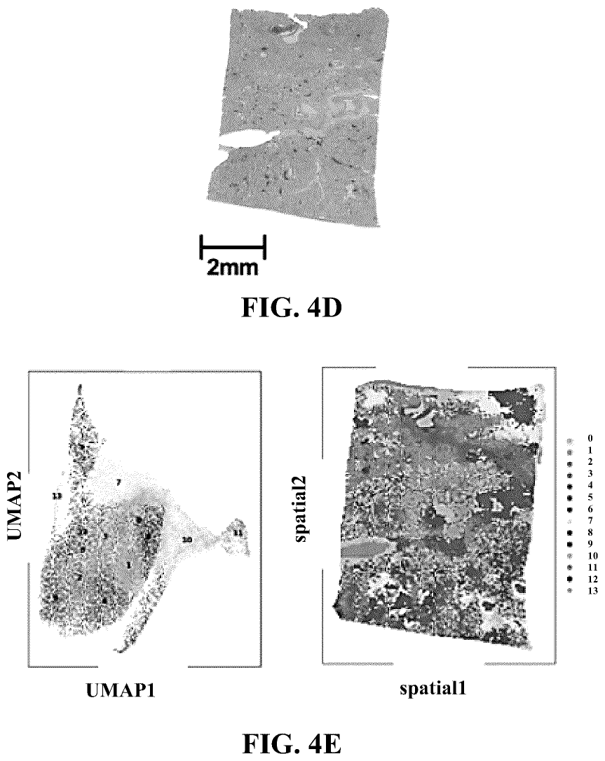

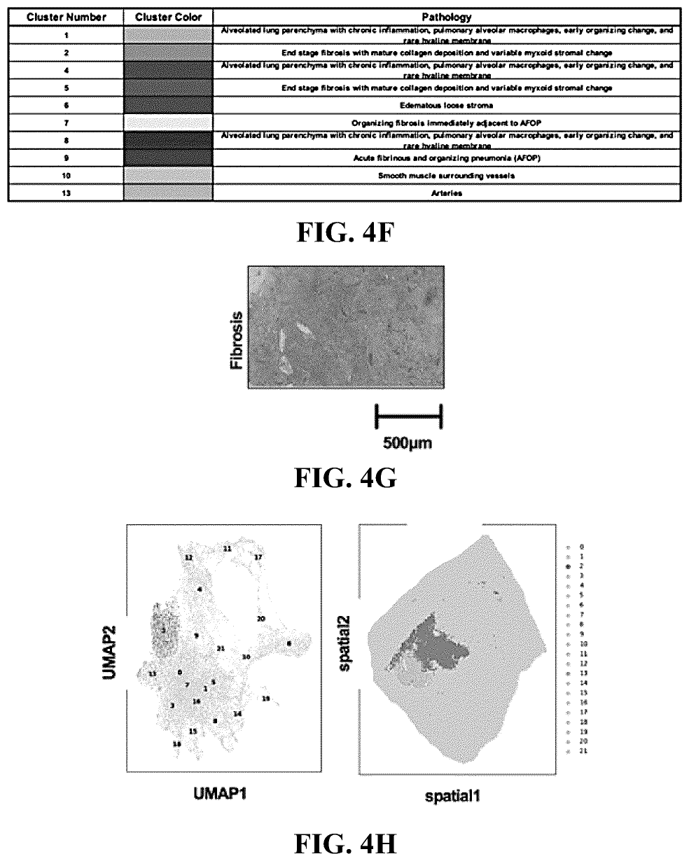

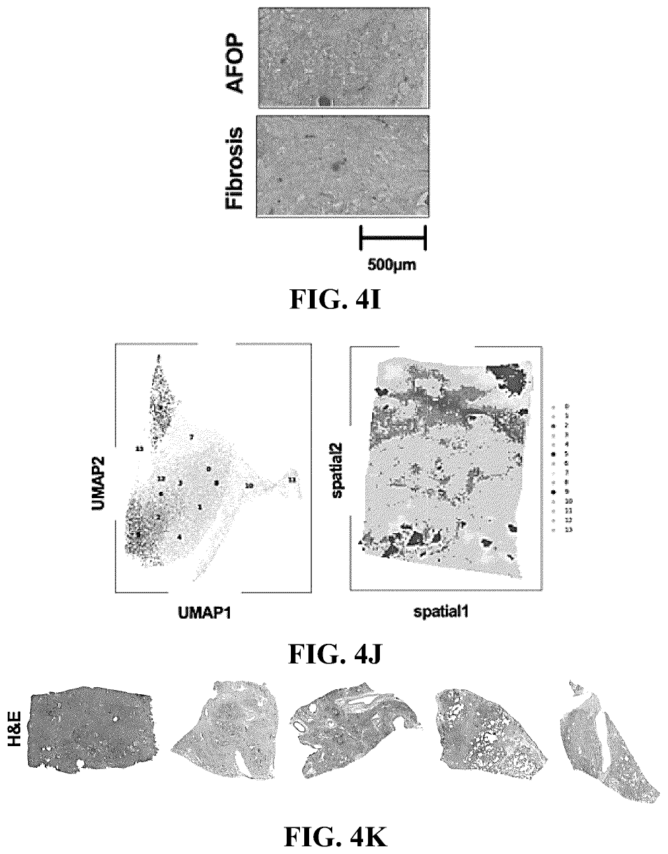

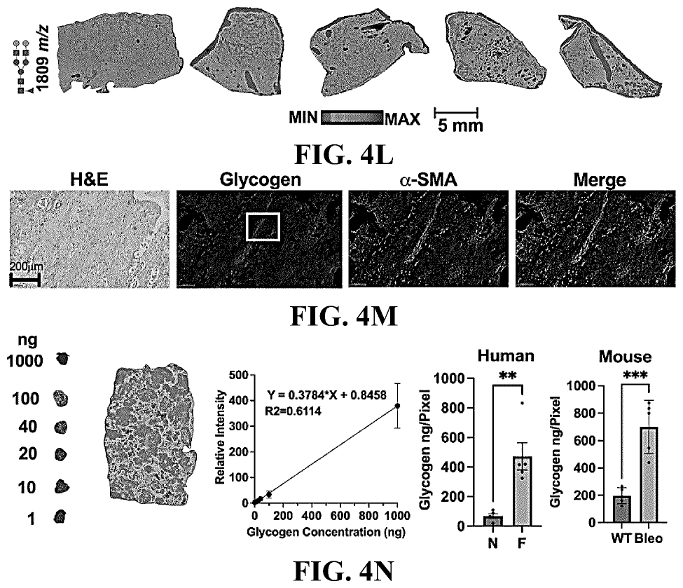

HDR-SC Reveals Major Histopathology of Human Lung Disease. To test whether HDR-SC analysis can identify pathological changes in human diseased tissue, we performed MALDI-MSI on lung tissue sections from multiple human idiopathic pulmonary fibrosis (IPF) patients and a single COVID-19 patient (FIGS. 3A, 3F, and 4A, 4D). For each human tissue section, HDR-SC identified different clusters between the IPF (FIGS. 3B and 4B) and COVID-19 (FIGS. 3G and 4E) patient samples. These clusters matched accurately to smooth muscle (cluster 0), end-stage fibrosis (cluster 5), diffuse alveolar damage (DAD) (cluster 6), mucin aggregates in the airway (cluster 10), and edematous loose stroma (cluster 11) (FIG. 3C) in the IPF patient tissue. In addition, clusters matched accurately to early (cluster 1), mid (cluster 0), and end-stage fibrosis (clusters 3 and 4) as well as DAD (clusters 6 and 8) and mucin aggregates in the airway (cluster 12) in the COVID-19 patient (FIG. 311). Notably, HDR-SC analysis accurately identified shared pathologies between IPF and COVID-19 tissues including DAD, end-stage fibrosis, and mucin aggregation (FIGS. 3D, 3E, 3I, and 3J). Histopathology analysis of additional IPF and COVID-19 tissues following HDR-SC, summarized in FIGS. 4C and 4F, also accurately identified end-stage fibrosis as a major histopathological feature of both lung diseases based on unique complex carbohydrate features (FIGS. 4G-4J).

Glycogen as a Clinical Hallmark of PF from Different Disease Origins. Pulmonary fibrosis (PF) is a severe and terminal disease that currently lacks a cure. There is a critical need to identify therapeutic vulnerabilities within PF for the future development of small molecule inhibitors. Based on HDR-SC, we performed metabolite enrichment analysis among each pixel within different annotated clusters and examined overrepresented carbohydrate features specific to fibrotic regions (FIG. 3K ). We identified a number of core fucosylated N-linked glycans upregulated among fibrotic regions between different patients (FIGS. 3K and 4K-4L). This finding supports previously published results that fucosylation is involved during fibrosis disease progression³⁸⁻⁴¹. Interestingly, we observed a number of glucose polymers appear in the overrepresented metabolite list which correspond to glycogen accumulation in the fibrotic region (FIGS. 3K-3L). Next, we further evaluated this spatial glycogen phenotype in additional IPF (n=3) and COVID-19 (n=1) tissues and confirmed that glycogen-rich regions correspond with fibrotic regions from adjacent H&E sections annotated by a board-certified clinical pathologist (FIGS. 3M-3N). To confirm that glycogen is uniquely accumulated in the myofibroblast, we performed colocalization analysis with immunofluorescence (IF) using an anti-glycogen antibody^{42,43} and an anti-alpha-smooth muscle actin (α-SMA) antibody⁴⁴. We observed robust co-localization between glycogen and α-SMA (FIGS. 3O and 4M), which supports the notion that glycogen is uniquely present in the myofibroblast cellular population within pulmonary fibrosis. These findings raise the interesting hypothesis that glycogen is a critical hallmark metabolite in PF and its role in PF should be further evaluated. To establish the absolute amount of glycogen in fibrotic and non-fibrotic regions of human and mouse specimens, we directly spotted increasing concentrations of purified liver glycogen as quantification standards adjacent to tissue sections followed by MALDI imaging (FIG. 4N). Using this approach, we demonstrated the dynamic linear range for glycogen by MALDI imaging from 1 ng to 1000 ng (FIG. 4N). Finally, we determined the absolute glycogen levels to be ˜1200 ng/pixel in fibrotic regions and ˜100 ng/pixel in non-fibrotic regions within human specimens (FIG. 4N).

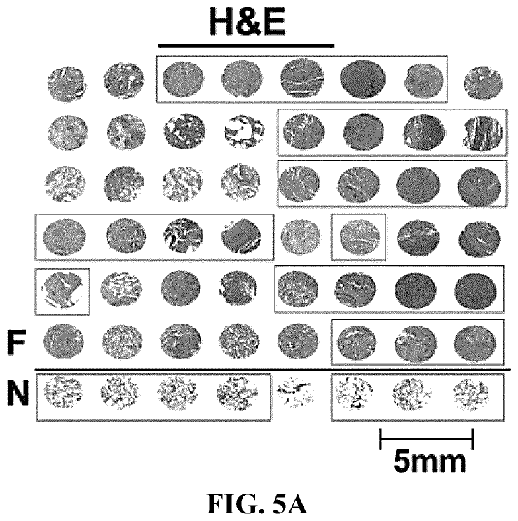

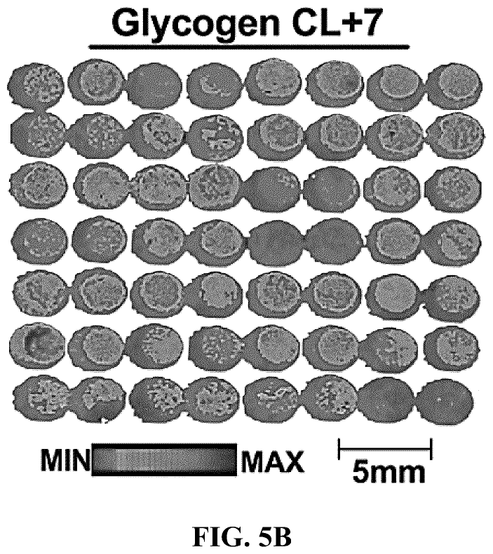

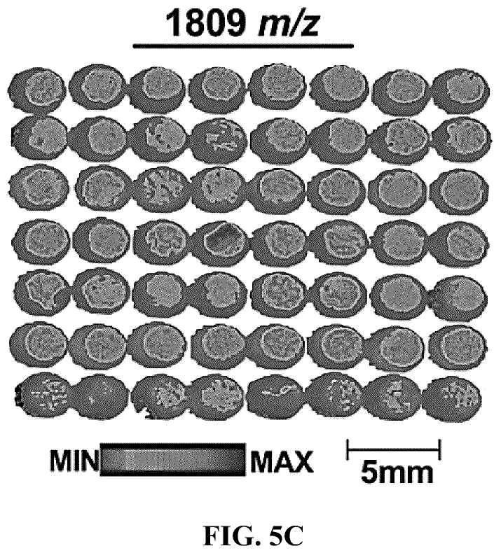

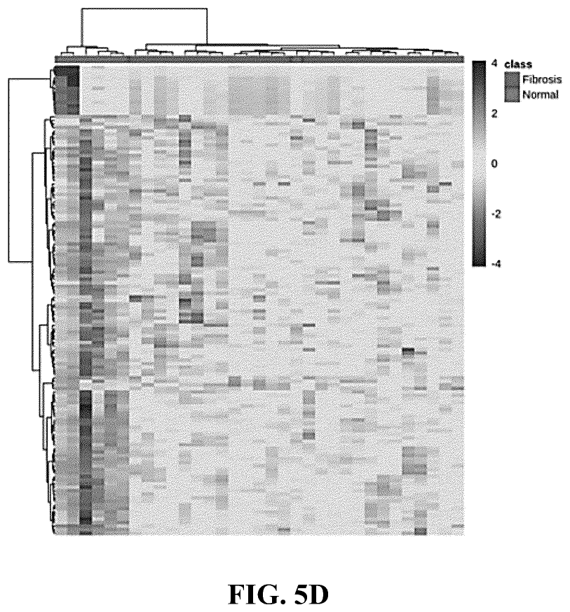

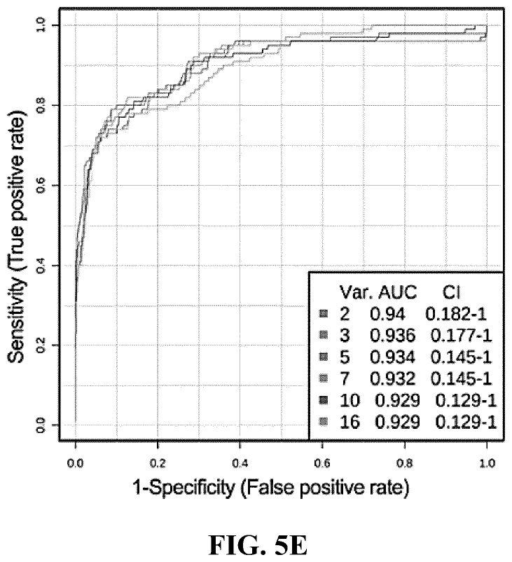

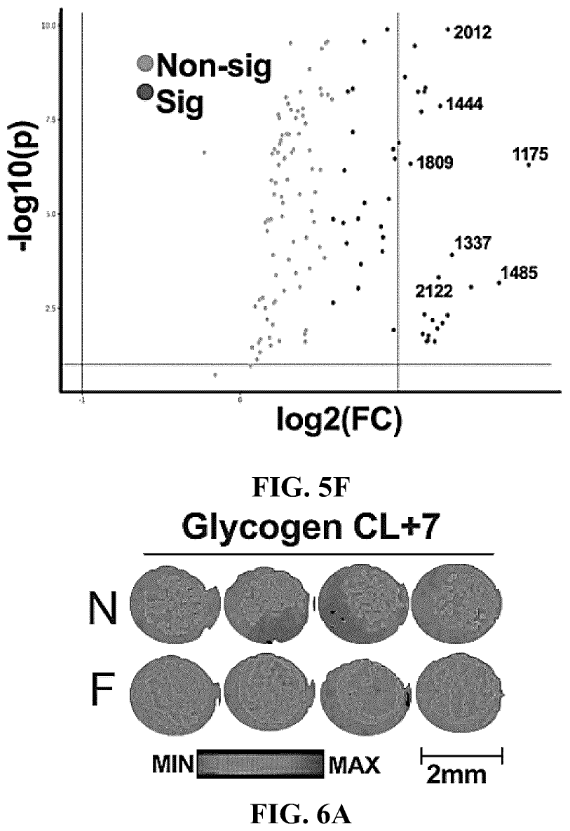

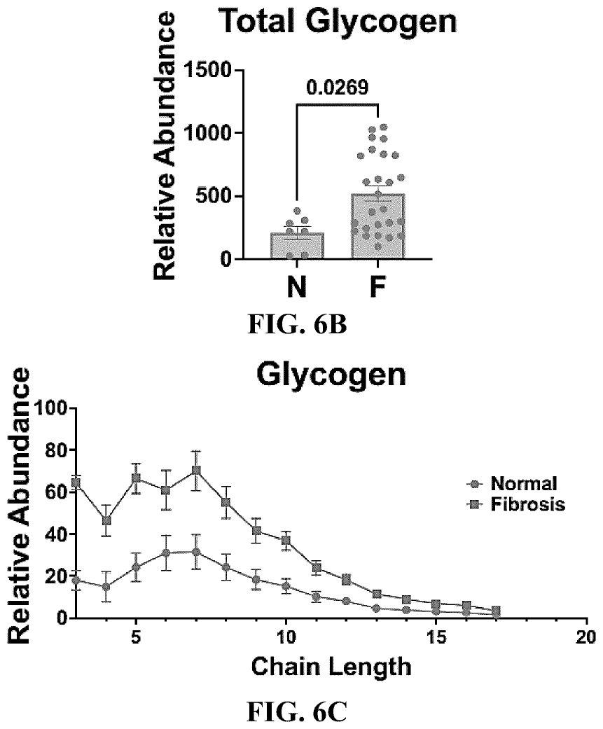

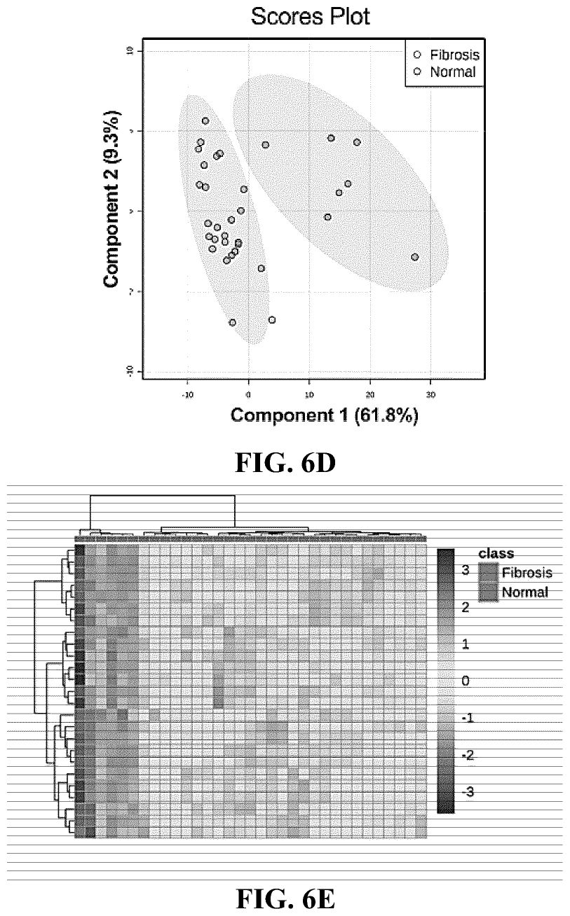

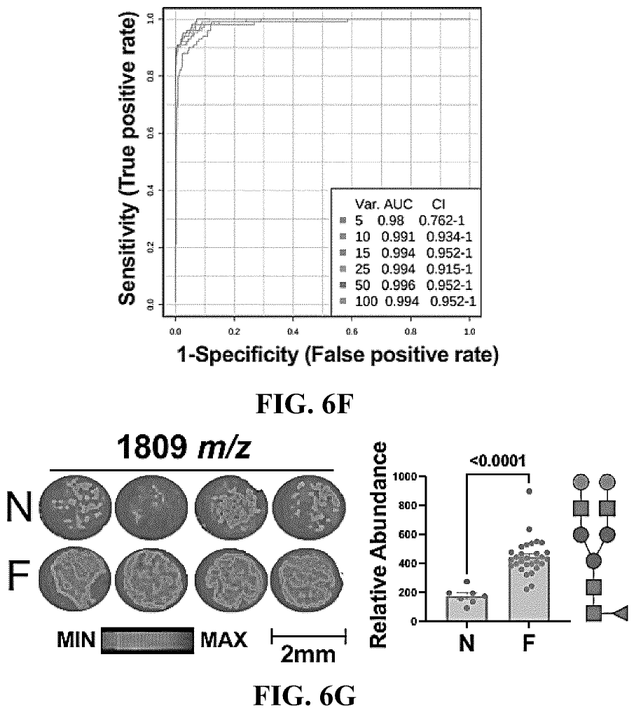

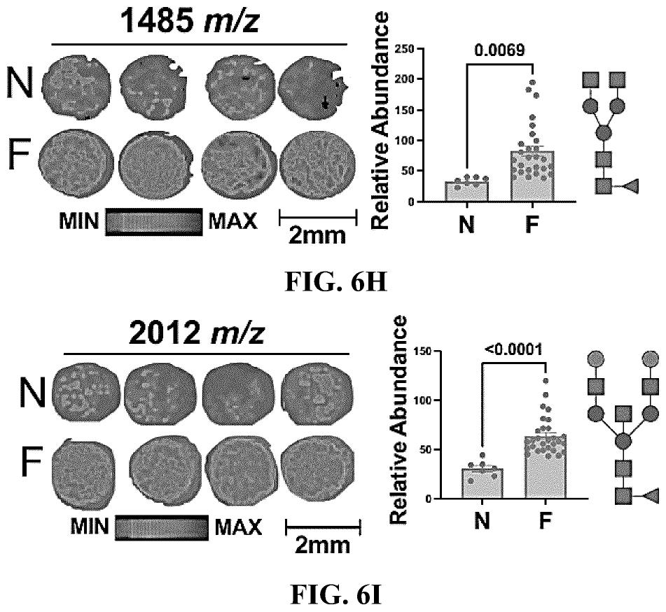

To further establish that glycogen is a metabolic hallmark of lung fibrosis, we purchased a commercial tissue microarray (TMA) comprised of both human fibrotic tissue cores (n=26) and normal adjacent lung resected at surgery (n=7) (FIG. 5A-5C). We performed MALDI-MSI on the cohort of human specimens from the TMA and performed glycogen and N-linked glycan analyses. In agreement with IPF/COVID-19 larger tissue analyses, we observed on average a 2.4-fold increase in glycogen within the fibrotic cores compared to the non-fibrotic normal lung controls (FIGS. 6A-6B). In addition, glycogen structure analysis shown significantly increases across the spectrum of glycogen chain lengths in fibrotic cores compared to normal lung tissue (FIG. 6C). Both partial least squares-discriminant analysis (PLS-DA) and unsupervised clustering heatmap analyses using glycogen-derived glucose polymers and N-linked glycans exhibit clear separation between fibrosis and normal samples (FIGS. 5D and 6D-6E). Further, we performed receiver operating characteristic (ROC) analysis to assess the clinical diagnostic ability of glycogen and N-linked glycans as predictive features of lung fibrosis. While glycogen alone shows an exceptional predictive AUC value of 0.94 (FIG. 5E), glycogen in combination with N-linked glycans shown an even better predictive AUC value of 0.996 (FIG. 6F). Finally, we also confirmed that core fucosylated N-linked glycans were upregulated in fibrotic tissue cores from the TMA similar to previous results in our IPF/COVID-19 patient samples (FIGS. 5F and 6G-6J). Collectively, our data supports the notion that glycogen associated N-linked glycans are clinical hallmarks of pulmonary fibrosis that warrant further studies into its therapeutic potentials.

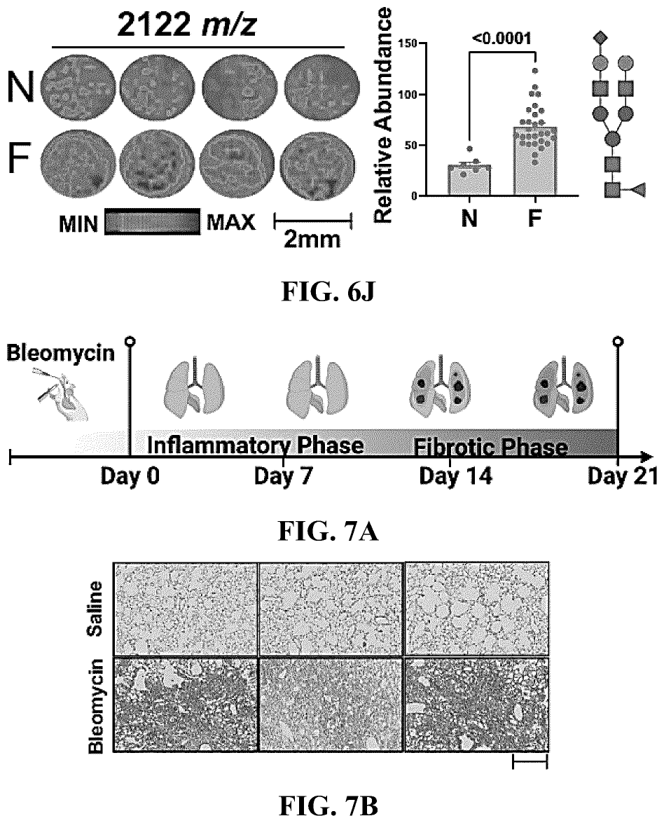

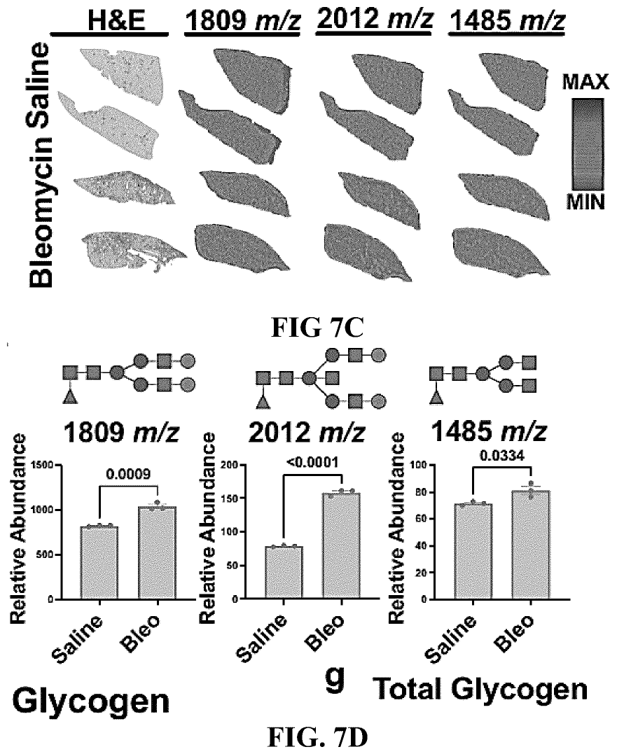

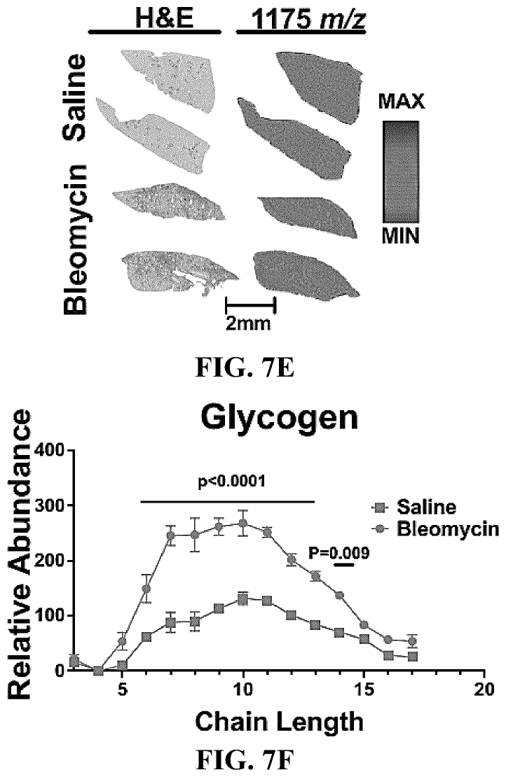

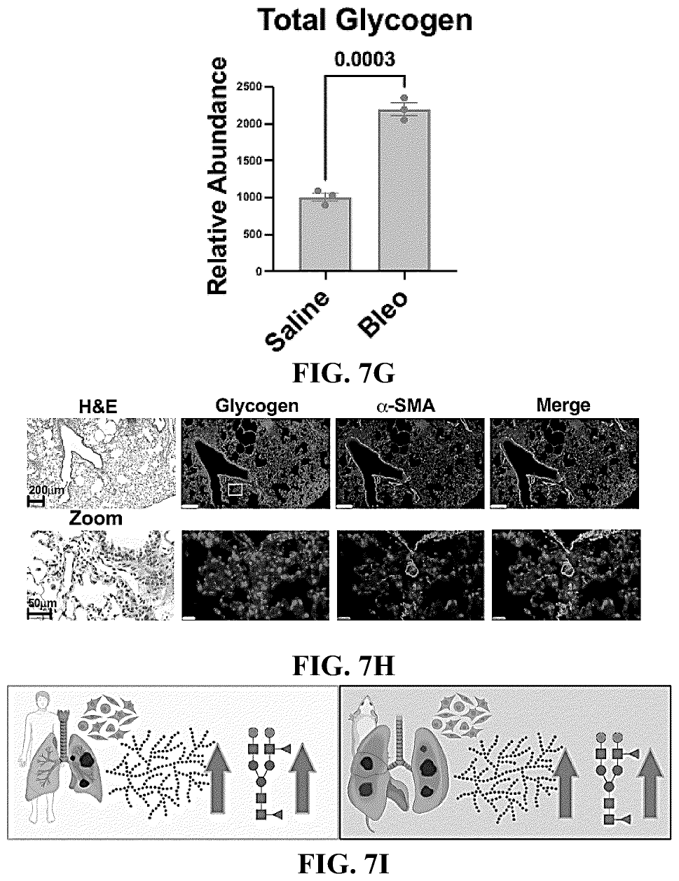

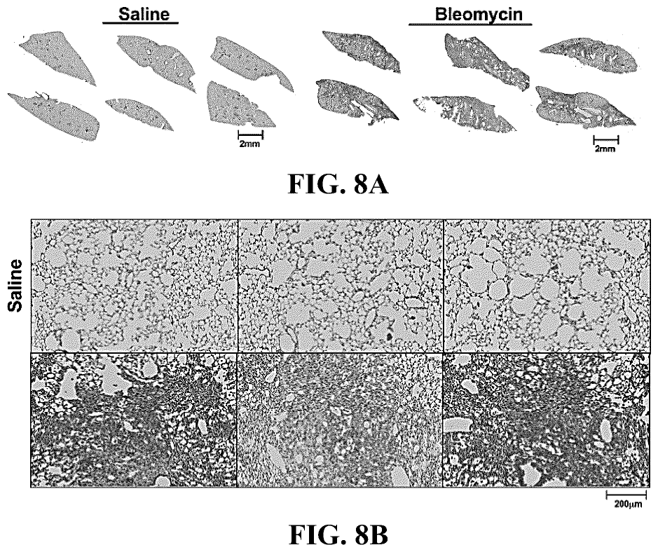

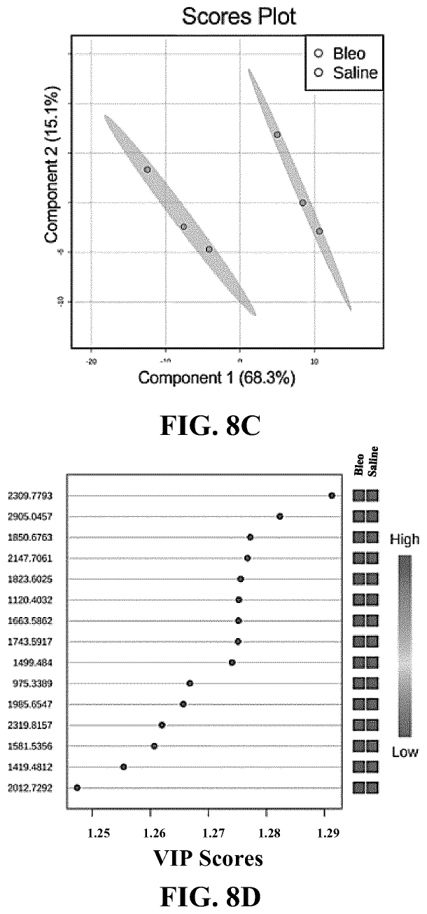

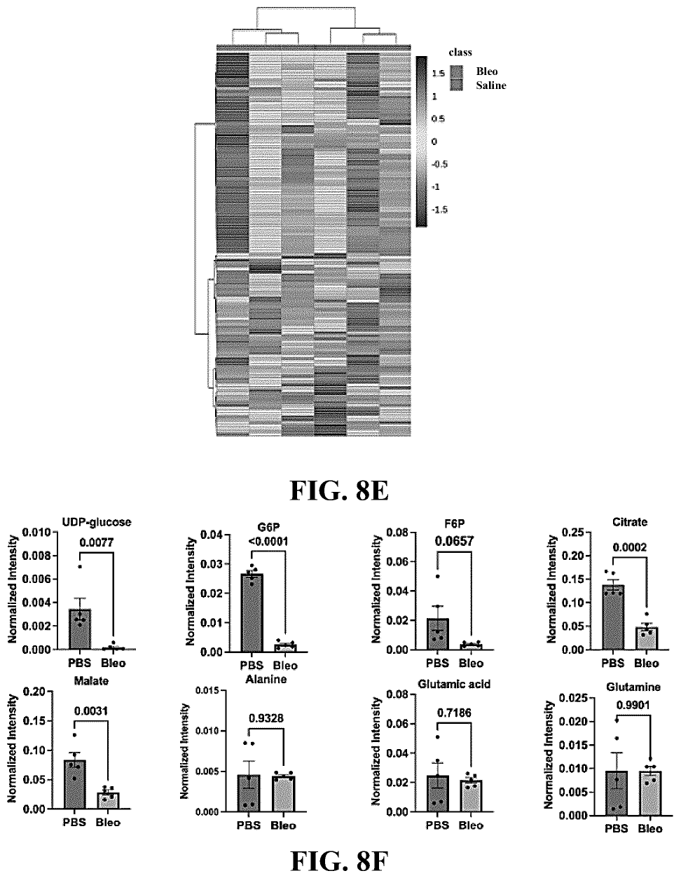

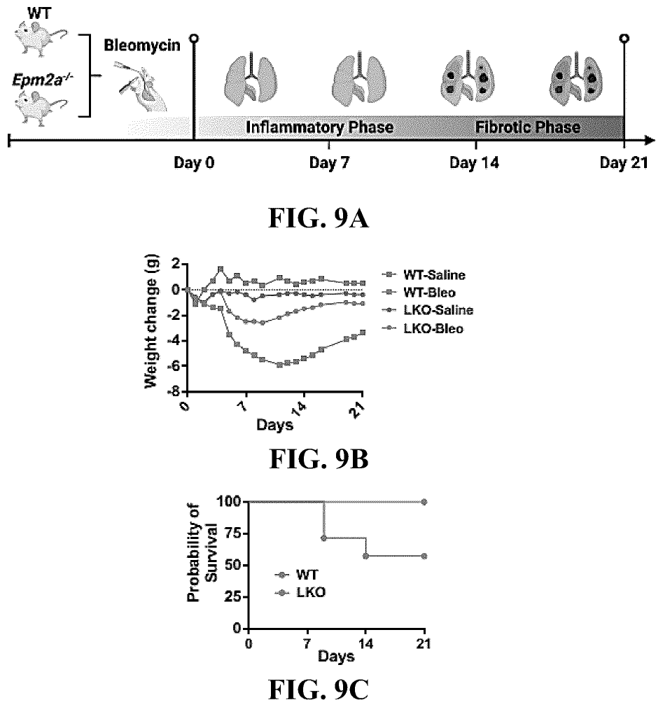

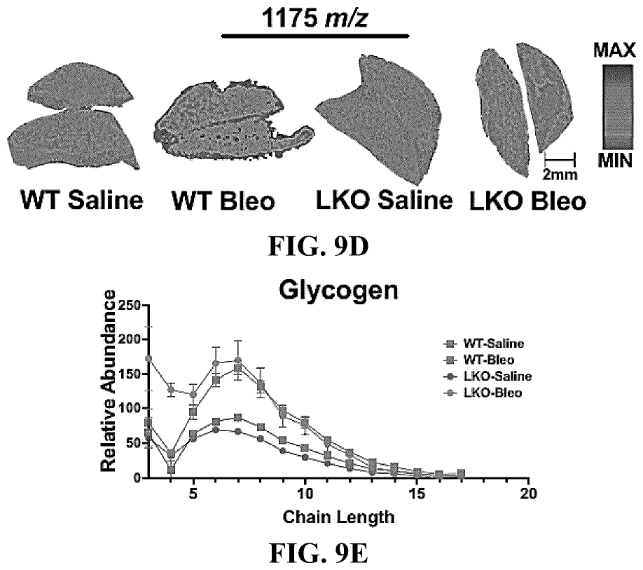

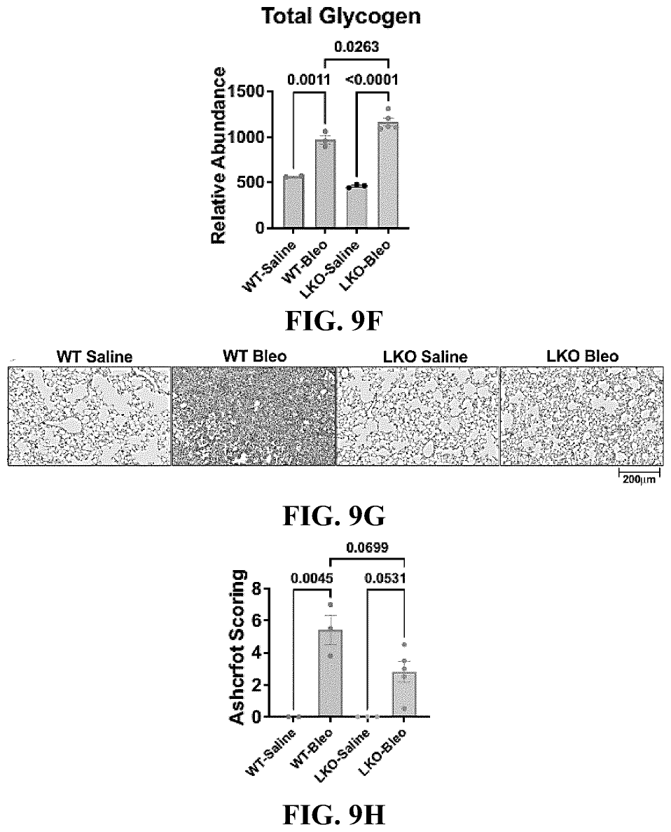

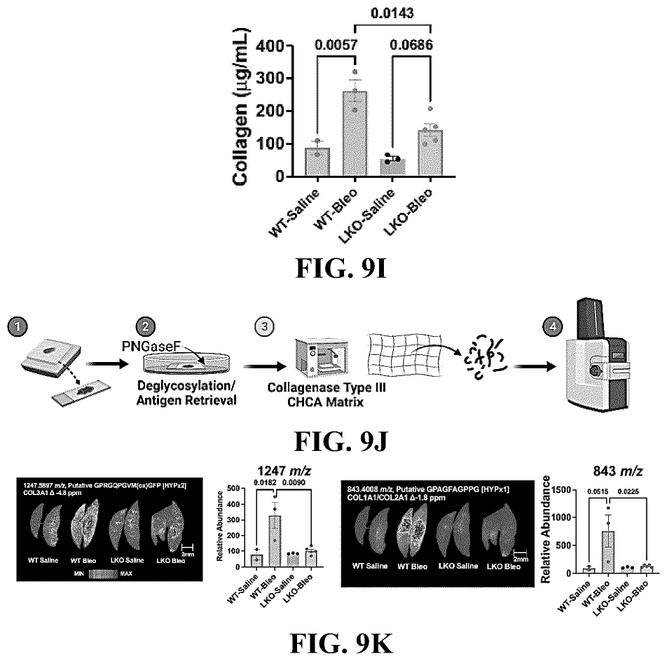

Bleomycin-Induced Pulmonary Fibrosis display Increased Glycogen. Based on human specimens, we identified extensive changes in N-linked glycan and glycogen accumulation in PF (FIG. 6B-6J). We have previously established that glycogen and N-linked glycans are metabolically channelled and glycogenolysis or glycogen utilization provides substrates for N-linked glycosylation^{43,45}. Therefore, we hypothesize that glycogen and N-linked glycan phenotypes observed in PF are linked and a critical step during the progression of fibrosis, and by preventing glycogen utilization we could inhibit fibrosis development, and, in theory, glycogen could be a potential therapeutic target for the treatment of fibrosis. To test this hypothesis, we utilized a common preclinical animal model of fibrosis resulting from intratracheal administration of bleomycin^{46,47}. Administration of bleomycin induces an initial dose-dependent acute lung injury in the first seven days, followed by the development of fibrosis over the next 14 days⁴⁷. First, we assessed whether mice with PF exhibit similar complex carbohydrate features compared to human fibrosis. Mice were intratracheally administered either bleomycin (0.3 U/kg) or saline and sacrificed 24 hours after day 21, followed by surgical removal of the lungs (FIG. 7A). Mice were weighed daily and monitored for signs of impaired mobility, reduction in size/body weight, difficulty breathing, and/or generalized cachexia. Bleomycin treatment generated the predicted fibrotic response by day 21, determined by H&E staining and pathological assessment (FIGS. 7B and 8A-8B). Lungs were then assessed by MALDI-MSI analysis to determine if mice with PF exhibit similar complex carbohydrate features to human fibrosis. Initial multivariate analyses demonstrate clear separation between saline and bleomycin treated mouse lungs by both PLS-DA and unsupervised clustering heatmap analysis (FIG. 9C-9E). Similar to human specimens, the bleomycin PF model displays increased core fucosylated N-linked glycans (FIGS. 7C-7D), and, more importantly, the bleomycin PF model exhibits a 2.4-fold increase in glycogen stores (FIGS. 7E-7G), similar to those identified in human specimens (FIG. 6G-6J). Also similar to human specimens, co-localization between glycogen and α-SMA was observed in the mouse model of PF (FIG. 711), suggesting glycogen is uniquely localized with myofibroblast cells. Finally, we performed targeted liquid-chromatograph mass spectrometry analysis using pooled PBS and bleomycin treated mouse lungs (FIG. 9F). Metabolites associated with glycogen metabolic pathways such as UDP-glucose and glucose-6-phosphate were significantly decreased at the onset of fibrosis in bleomycin treated mice (FIG. 9F).

Interestingly, amino acids such as alanine, glutamic acid, and glutamine remain unchanged between the two groups (FIG. 9F). Collectively, our data suggest similarities in complex carbohydrate metabolism between the PF model and human disease (FIG. 7K), and thus justify the bleomycin-induced PF preclinical model to study glycogen and N-linked glycan metabolism in PF.

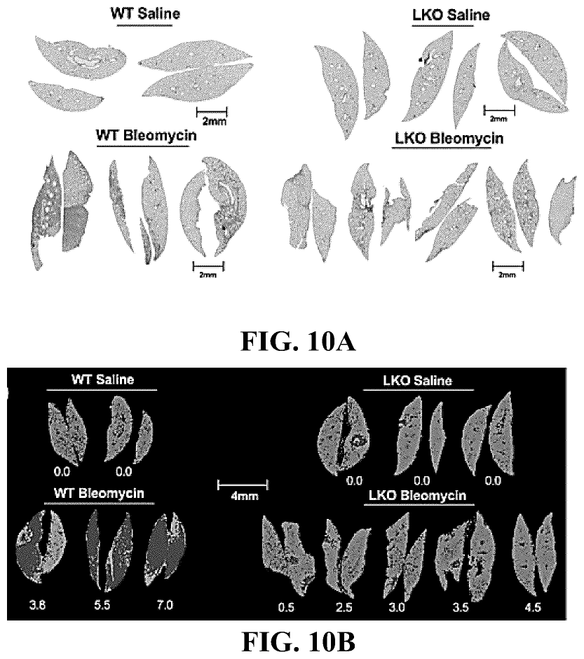

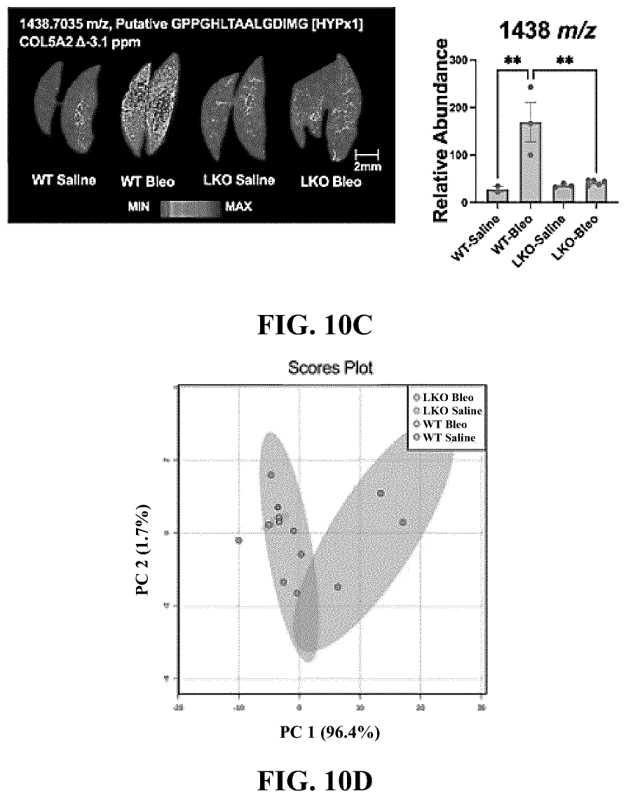

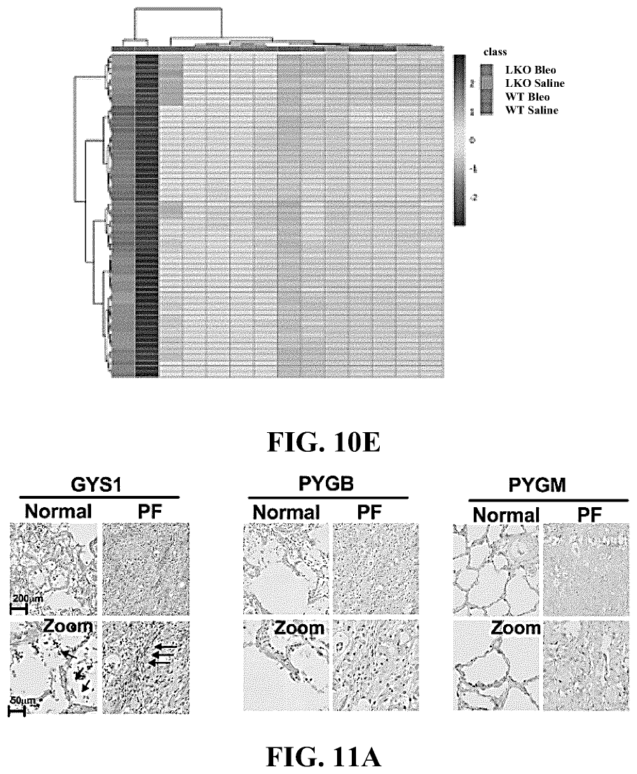

Inability to Utilize Glycogen blunts Fibrosis Development In Vivo. To test whether glycogen utilization is necessary for fibrosis disease progression, we employed a mouse model of glycogen storage disease lacking the glycogen phosphatase laforin (Epm2a^{-/-}). Glycogen dephosphorylation is required for efficient glycogenolysis and the release of glucose-6-phosphate by glycogen phosphorylase^{48,49}. Laforin deficiency results in hyperphosphorylated glycogen aggregates that are inaccessible to glycogen degradation enzymes^{15,50-52}. Thus, the mice accumulate glycogen that they are unable to degrade. We administered bleomycin to both WT and Epm2a^{-/-} mice (LKO) and collected lungs 24 hours after day 21 post-treatment similar to the previous experiment (FIG. 9A). During the experiment, bleomycin treated LKO mice did not lose as much weight compared to the bleomycin treated WT mice (FIG. 9B). Further, three WT mice were euthanized during the treatment window due to >20% weight loss, while none of the LKO mice met the criteria for euthanasia (FIG. 9C). Following euthanasia and lung dissection, we performed H&E histopathological staining and MALDI-MSI on the lungs of all four cohorts of mice (FIGS. 10A and 10B). Next, we performed targeted analysis in only the fibrotic regions between both WT and LKO mice guided by H&E image of an adjacent lung section. As predicted, we observed a significant buildup of glycogen stores in the LKO bleomycin treated cohort, due to the inability to utilize glycogen in this mouse model (FIG. 9D-9F). Increased glycogen within the LKO bleomycin cohort correlated with a 2-fold reduction in fibrosis burden when compared to WT mice treated with bleomycin based on histopathological assessments and Ashcroft scoring (FIG. 9G-9H).

Fibrosis progression relies on remodeling of the lung extracellular matrix proteins⁵³. Primarily, extensive collagen deposition within the alveolar airways provides the necessary structural and mechanical support for the chronic proliferation of diseased fibroblasts⁵⁴. While mechanisms behind increased collagen synthesis during PF remain to be elucidated, it is known that the degree of collagen lattice correlates with disease severity⁵⁵. In our study, we performed total collagen quantitation from the bronchoalveolar lavage fluid (BALF) and observed similar trends between LKO and WT bleomycin treated cohorts (FIG. 9I). To confirm that the reduction of collagen is from the fibrotic regions, we performed spatial proteomics imaging using collagenase digestion to specifically interrogate collagen isoforms within the fibrotic regions^{56,57} (FIG. 9J). Similar to established biology, we found significant enrichment of collagen peptides within the fibrotic regions of the lung that correlated with the Ashcroft score (FIG. 10B). Interestingly, LKO animals treated with bleomycin have much lower levels of collagen content within the fibrotic regions compared to WT bleomycin cohort when normalized to number of pixels within the fibrotic region (FIG. 10B). Further, we saw significant decreases in peptides derived from collagen 1A1/2A (FIG. 9K), 3A1 (FIG. 9K), 5A2 (FIG. 10C), suggesting blocking glycogen utilization results in the downregulation of more than one type of collagen. This phenotype is further validated by principal component analysis (PCA) and unsupervised clustering heatmap analyses, showing global reduction collagen peptides within the fibrotic regions of LKO mice (FIGS. 10D-10E). Collectively, multidimensional spatial metabolomics and proteomics analyses suggest collagen deposition during PF is linked to glycogen metabolism and an orthogonal method to confirm the lack of endpoint fibrosis in LKO mice.

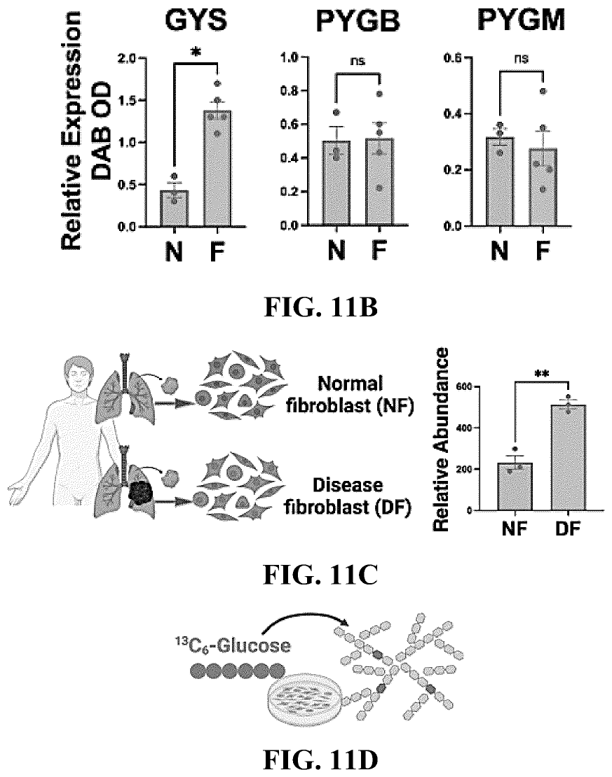

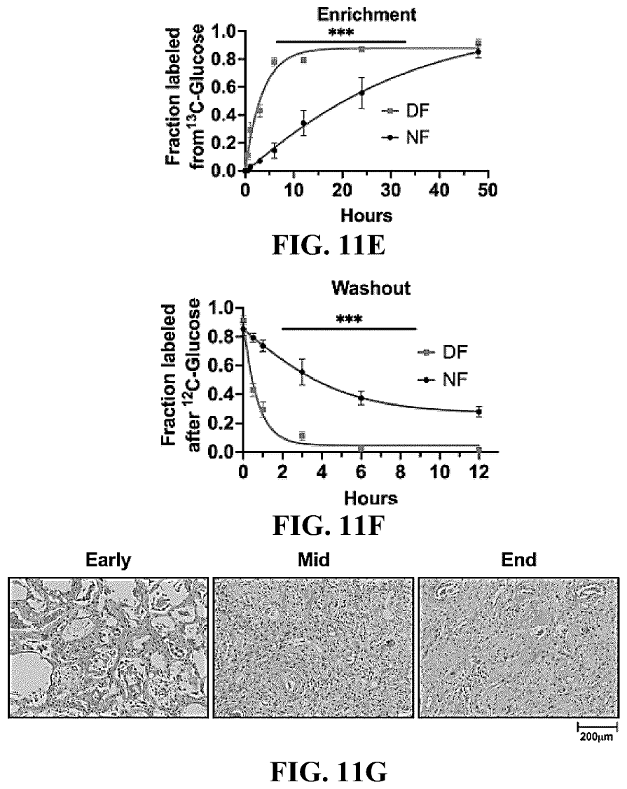

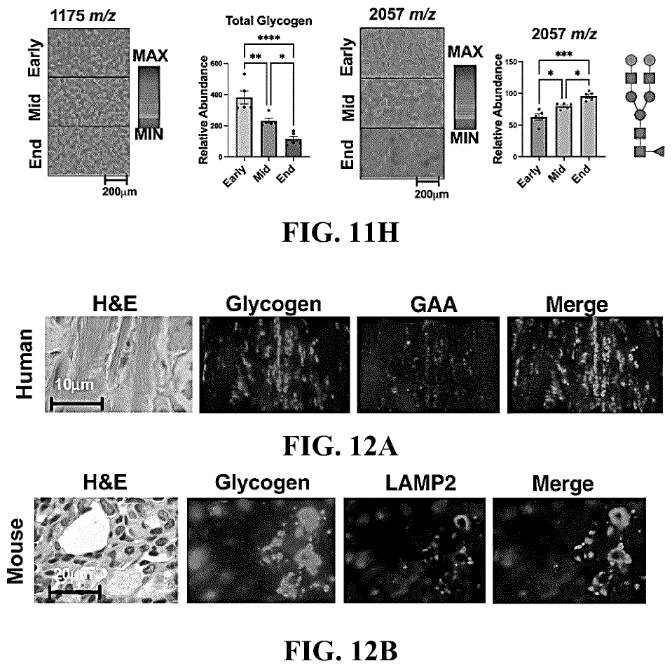

Acid Alpha-Glucosidase (GAA) Drives Glycogen Utilization in PF. Elucidating the route of glycogen utilization is needed to improve our understanding of PF biology and future translational opportunities. First, we performed immunohistochemical staining to assess the levels of glycogen synthase (GYS) and glycogen phosphorylase muscle and brain isoform (PYGM and PYGB respectively) at the protein level¹⁷. We observed increased GYS protein expression within the fibrotic regions when compared with the non-diseased regions from the same tissue section (FIGS. 11A and 11B). However, we did not observe similar increases for PYGM and PYGB at the protein level (FIGS. 11A and 11B). To further assess glycogen metabolism in disease fibroblasts, we performed both total pool and ¹³C-glucose traced glycogen in both normal fibroblasts and disease fibroblasts isolated from normal and IPF patients, respectively, using previously established mass spectrometry methods^{58,59}. Similar to patient specimens, diseased fibroblasts display a glycogen-rich phenotype (FIG. 11C). We then performed ¹³C-glucose to glycogen enrichment followed by a washout analyses that represents the rate of glycogen biosynthesis and degradation, respectively. Diseased fibroblasts display significantly higher enrichment and washout rates compared to the normal fibroblast (FIGS. 11D-11F), suggesting increased metabolic demand for glycogen in diseased fibroblasts with increased glycogen biosynthesis and degradation. To further support glycogen is important during fibrosis progression, we identified regions of early, mid, and end-stage fibrosis from our existing patient tissue sections (FIG. 11G). in agreement with our hypothesis, there is a stepwise decrease in glycogen stores from early to end-stage fibrosis (FIG. 11H). In contrast, we observed stepwise increases in biantennary N-linked glycan in tissue regions progressing from early to end-stage fibrosis (FIG. 11H). Collectively, stage-dependent decreases in glycogen patient tissue sections offers additional evidence to support our hypothesis that glycogen is utilized during fibrosis disease progression.

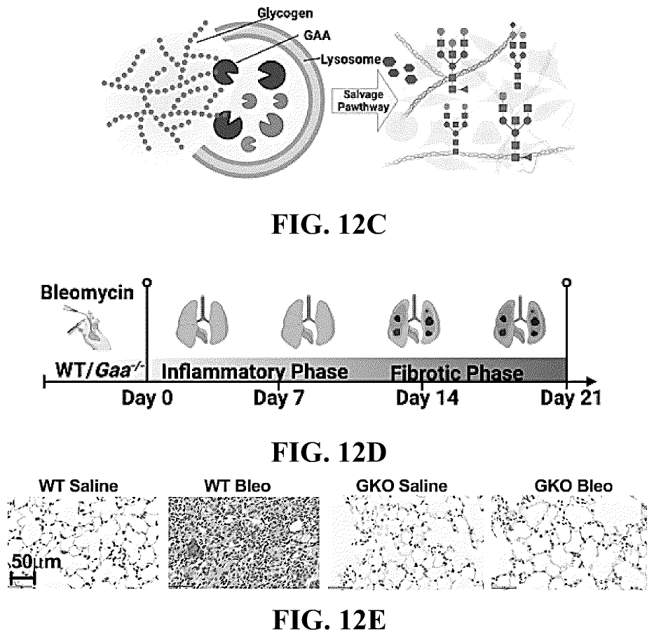

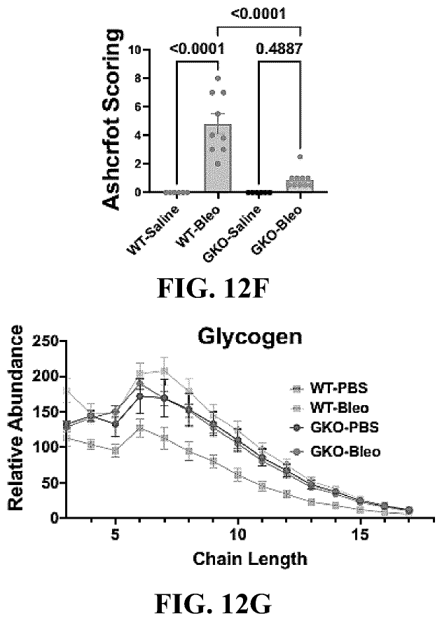

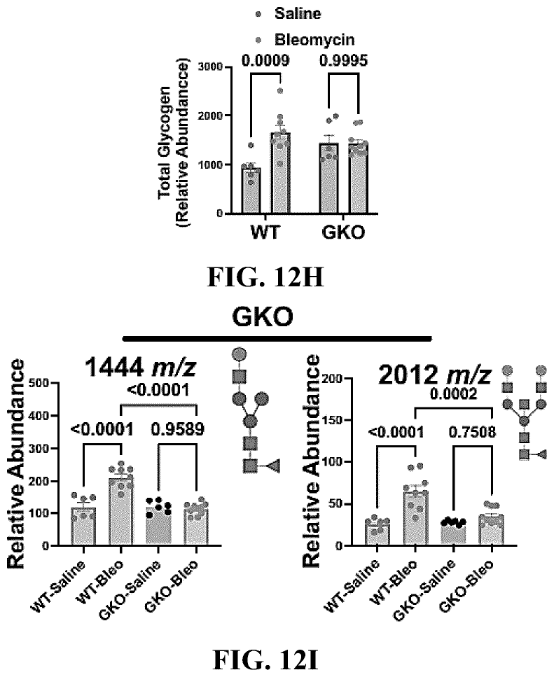

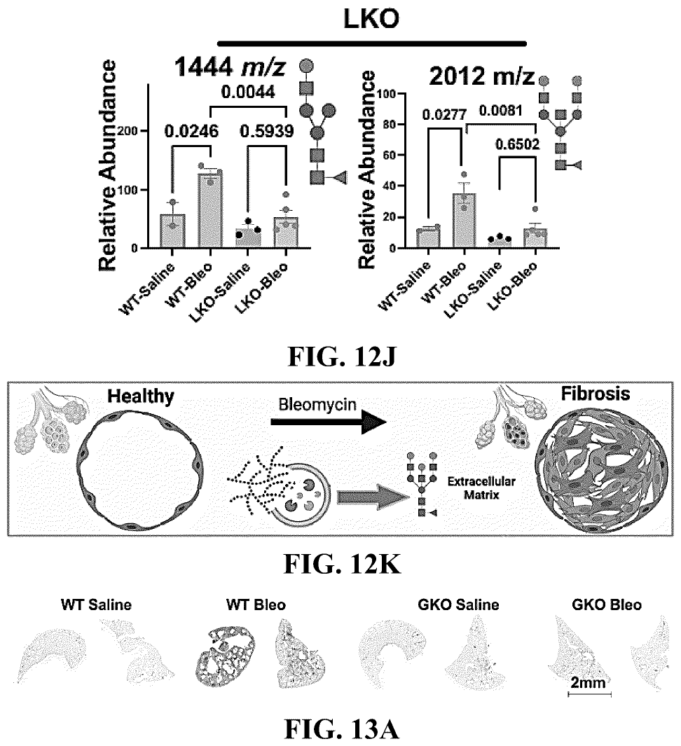

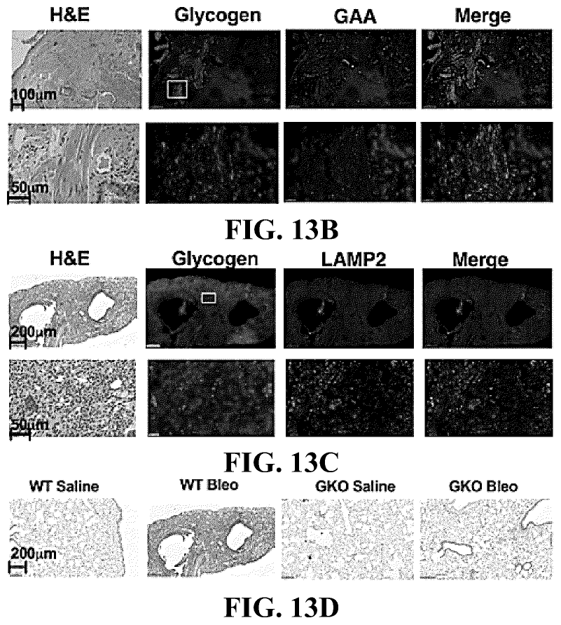

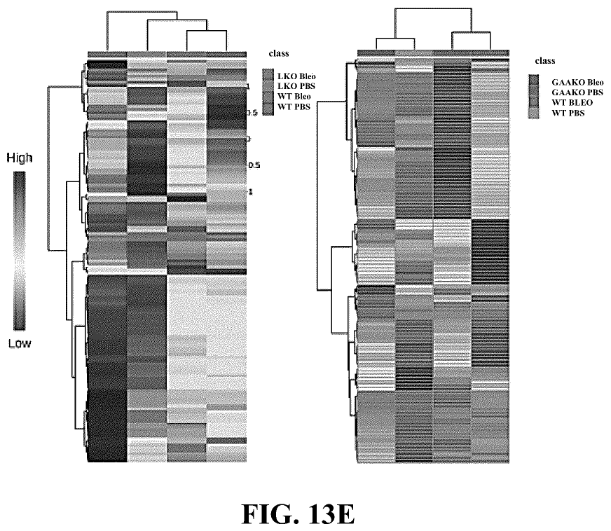

Increased rate of glycogen utilization without increases in GP protein expression, a key glycogen degradation enzyme, suggested an alternative route of glycogen degradation in the diseased fibroblasts. In our IF analysis using the anti-glycogen antibody, we observed round/puncta glycogen within the cytoplasm of myofibroblasts (FIGS. 12A, 12B, and 13B). This pattern has been described during glycogen phase separation⁶⁰ and its targeting into lysosomes^{61,62}. Since GAA is the lysosomal enzyme responsible for glycogen degradation (FIG. 12C)^{63,64} we performed co-localization analysis of GAA and glycogen using IF in human patient specimens. Extensive colocalization between glycogen and GAA suggests lysosomes are the site of glycogen utilization in PF (FIG. 12A). To confirm this phenotype in mice, we performed the same co-localization analysis using the lysosomal marker LAMP2. Similar to human results, mouse PF exhibited extensive co-localization between glycogen and LAMP2 (FIGS. 12B and 13C), further supporting that glycogen is utilized in the lysosome in both human and mouse PF.

The lysosome participates in the intracellular salvage pathways providing substrates for complex carbohydrate production (FIG. 12C). Given this known channeling, we hypothesized that glycogen utilization by GAA in the lysosomal salvage pathway may be necessary for fibrosis disease progression. Therefore, we employed a mouse model lacking GAA (Gaa^{-/-}). We administered bleomycin to both WT and Gaa^{-/-} mice (GKO) and collected lungs 21 days post-treatment similar to the previous experiment (FIG. 12D). Following euthanasia and lung dissection, we performed H&E histopathological staining and MALDI-MSI on the lungs of all four cohorts of mice (FIG. 13A). Strikingly, GKO mice exhibited nearly 5-fold reduction in fibrosis burden when compared to WT mice treated with bleomycin based on histopathological assessments and Ashcroft scoring (FIGS. 12E-12F and 13D). Interestingly, we observed high basal level glycogen in the GKO saline treated cohort, but glycogen did not further increase in the bleomycin treated cohort, possibly due to the lack of endpoint fibrosis formation (FIG. 12H).

To confirm that the lysosomal salvage pathway is compromised in GKO and LKO mice, we performed MALDI MSI to assess the N-linked glycome of the lung in both GKO and LKO cohorts of bleomycin and saline treated animals. We observed broad spectrum reduction in a wide variety of N-linked glycans in both GKO and LKO bleomycin treated lungs when compared to the WT bleomycin treated cohort (FIG. 11E), and the effect size was much greater in the GKO cohort. For example, N-linked glycans 1444 and 2012 m/z did not show increases in bleomycin treated GKO mice compared to GKO saline treated mice (FIG. 12I) but trending slightly higher in the LKO cohorts within the same comparison (although P value is greater than 0.05). Collectively, these preclinical data from two animal models of pulmonary fibrosis support the hypothesis that glycogen utilization through the lysosomal salvage pathway is critical for fibrosis development (FIG. 12K) and in agreement with the metabolic phenotype identified in human PF specimens.

### Discussion

In this study, we applied multidimensional spatial metabolomics analyses to demonstrate application for histopathology prediction and biomarker identification in human and mouse diseased tissues. We chose spatial metabolomics analysis of complex carbohydrates for a number of reasons: First, the ability to utilize stored FFPE tissue samples significantly increases accessibility to a variety of human disease clinical specimens^{34,65}. Second, complex carbohydrate metabolism is intimately connected to glucose utilization and spans multiple cellular compartments¹⁴. Third, complex carbohydrates are critical structural metabolites for cell-cell adhesion, signaling, and extracellular matrix remodeling^{14,66}. Fourth, aberrant complex carbohydrate metabolism drives pathogenesis in multiple human diseases including neurodegeneration^{67,68}, type II diabetes⁶⁹, cardiovascular disease⁷⁰, and several types of cancer^{71,72}. In this study, we demonstrate HDR-SC and biomarker prediction using spatial metabolomics datasets. HDR-SC accurately captured fibrotic regions in multiple tissue sections analyzed when matched to histopathology annotation. Further, differential feature analysis identified biomarkers that highlight a potential role for glycogen utilization during fibrosis disease progression. Using two separate mouse model of fibrosis, we demonstrate that the inability to utilize glycogen leads to significantly blunted N-linked glycan and collagen content and fibrosis development in vivo.

MALDI-MSI is a high-dimensional dataset, similar to the scRNAseq format⁷³ wherein complex carbohydrates are biological features stored within each pixel, similar to gene expression levels within each cell. However, there are several important differences. First, each pixel from MALDI-MSI is recorded with precise X-Y coordinates to allow true spatial mapping of anatomical regions. Second, multiplexed glycogen and N-linked glycan MALDI-MSI takes advantage of a large collection of well-annotated human clinical specimens with decades of clinical metadata in the form of FFPE. The ability to utilize FFPE tissue also bypasses the need to collect fresh tissue from surgery for human clinical studies. The in situ nature of MALDI-MSI also avoids lengthy and potentially destructive cell isolation steps, thus preserving anatomical regions. Of note, many histopathological features such as end-stage fibrosis and mucin patches contain large fractions of non-cellular features. For example, the presence of mucin within airways and in regions of honeycomb change contain abundant glycoproteins. These features are often lost in single-cell analyses but are preserved by the MALDI-MSI workflow. To this end, what MALDI-MSI lacks in cellular information could be complimented with scRNAseq, multiplexed ion beam imaging by time of flight (MIBI-TOF), or co-detection by indexing (CODEX) analyses⁷⁴. Co-analysis or vertical integration with single-cell techniques would enhance cellular metabolic origins that are related to the etiologies of diseases.

Pulmonary fibrosis (PF) is the long-term consequence of genetic mutations or acute damage to the lung from environmental exposures, including radiation/chemotherapy^{75,76}, acute respiratory distress syndrome (ARDS)⁷⁷, cancer⁷⁸, and, more recently, severe lung disease caused by the SARS CoV-2 virus (COVID-19)⁷⁹. In some cases, PF arises from undefined causes and is classified as IPF⁸⁰. The pathology of PF includes remodeling of the alveolar regions with excessive matrix production such as N-glycans and collagen by fibroblasts, myofibroblasts, and mucin aggregation that lead to severe thickening of the alveolar septa of the lung^{81,82}. PF patients suffer from poor gas exchange and subsequently low tissue oxygenation that leads to other comorbidities and death⁸³. Our pipeline identified unique subclasses of complex carbohydrates enriched in myofibroblasts of PF patient samples. In our analyses, core fucosylated N-glycans and glucose polymers from glycogen are differentially enriched in the myofibroblasts of PF. Using a separate TMA containing fibrosis and normal tissues, we demonstrate exceptional binary predictability of N-glycans and glycogen for the diagnosis of fibrosis. Future studies should focus on the predictability in multivariant pathologies such as acute fibrinous and organizing pneumonia (AFOP), DAD, and mucinous lesions. We anticipate that artificial intelligence (AI) would aid in the development process of implementing spatial metabolomics to digital pathology.

Current PF treatment relies on steroids that only temporarily delay fibrosis progression⁸⁴, and there are no effective treatments to reverse the clinical course of the disease, resulting in permanent loss of lung function⁸⁰. MALDI-MSI analyses of human pulmonary disease specimens revealed a glycogen-rich phenotype within the fibrotic regions. Interestingly, mouse models of PF through bleomycin-induction also displayed a similar glycogen-rich phenotype.

Through a series of molecular, cellular, and animal modeling experiments, we concluded that there is an increased metabolic demand for glycogen metabolism in diseased fibroblasts and highlighted that glycogen utilization by GAA through the lysosomal pathway is a critical metabolic process that occurs during pulmonary fibrosis. Mice deficient in GAA did not form PF after bleomycin treatment. The lysosomal salvage pathway provides substrates for the synthesis of proteoglycans⁸⁵ and N-linked glycans⁸⁶ that are critical component of the extracellular matrix. Coincidentally, myofibroblasts drive extracellular remodeling of PF⁴⁴, and increased glycogen utilization through the lysosomal salvage pathway would support the metabolic demand for extracellular matrix remodeling. This hypothesis is supported by the result demonstrating that Gaa^{-/-} mice blunted the increase in N-linked glycans after bleomycin treatment. We propose that this phenotype is shared between PF from different disease origins. These results are particularly exciting, as recent developments of glycogen targeting therapeutics demonstrate efficacy in preclinical models of glycogen storage diseases⁸⁷⁻⁹⁰, these include multiple modalities that targeting GYS through small molecule inhibition or anti-sense oligonucleotides with some already being tested in patients⁹¹. Repurposing these compounds could offer an additional treatment avenue for patients suffering from PF.

### Example 2: Laforin Inhibition Decreases Fibrosis-Related Gene Expression in Diseased Human Lung Fibroblasts

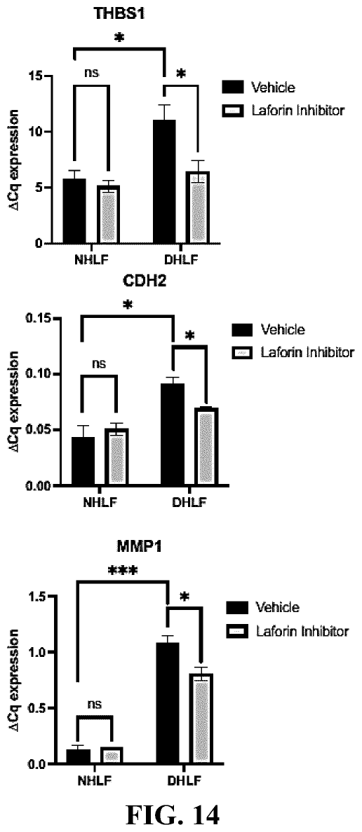

Normal human lung fibroblasts (NHLF) or diseased human lung fibroblasts (DHLF) derived from idiopathic pulmonary fibrosis patients were treated for two days with a laforin inhibitor (or vehicle control). The laforin inhibitor used in this Example was compound “L319-21-M50,” as described in U.S. Patent Application Publication No. 2018/0170862. Following treatment, mRNA was harvested from the cells and gene expression was assessed. As shown in FIG. 14, fibrosis gene expression in normal and fibrotic human fibroblast following laforin inhibition show disease-specific expression patterns being rescued by laforin inhibition.

### Example 3: Treatment of Pulmonary Fibrosis Via Administration of Glycogen Phosphatase, Acid Alpha-Glucosidase (GAA), Glycogen Synthase (GYS), and Glycogen Phosphorylase (GP) Inhibitors

In view of the above-discussed findings and results in Examples 1 and 2 and the known roles of glycogen phosphatase, GAA, GYS, and GP in glycogen synthesis and/or utilization, the efficacy of certain glycogen phosphatase inhibitors, GAA inhibitors, GYS inhibitors, and GP inhibitors with respect to treating pulmonary fibrosis (PF) in a subject having or at risk of having PF will be examined.

### Glycogen Phosphatase Inhibitors

In future studies the efficacy of other glycogen phosphatase inhibitors besides L319-21-M50 examined in Example 2 with respect to treating PF in a subject having or at risk of having PF will be examined. Glycogen phosphatase inhibitor compounds to be tested include:

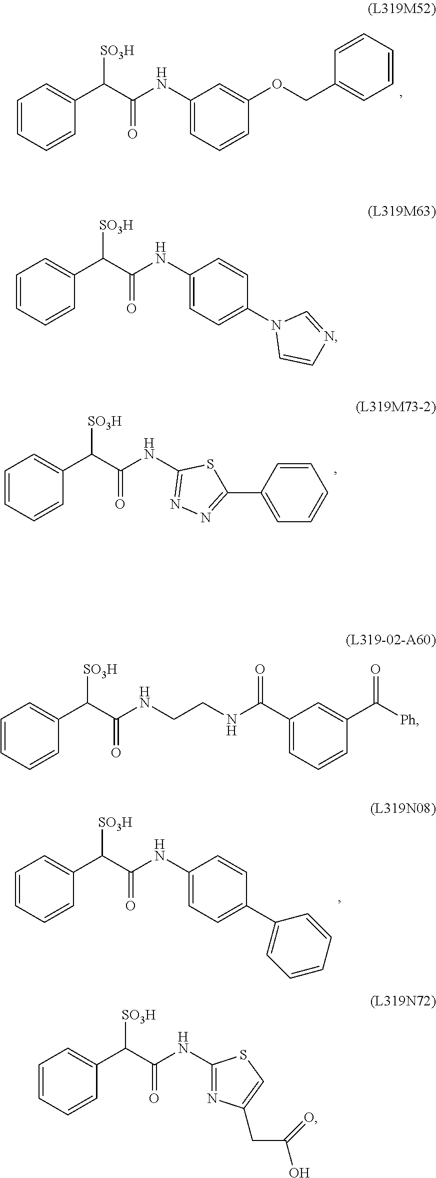 [SMILES: O=C(Nc1cccc(OCc2ccccc2)c1)C(c1ccccc1)S(=O)(=O)O ; O=C(Nc1ccc(-n2ccnc2)cc1)C(c1ccccc1)S(=O)(=O)O ; O=C(Nc1nnc(-c2ccccc2)s1)C(c1ccccc1)S(=O)(=O)O ; O=C(NCCNC(=O)C(c1ccccc1)S(=O)(=O)O)c1cccc(C(=O)c2ccccc2)c1 ; O=C(Nc1ccc(-c2ccccc2)cc1)C(c1ccccc1)S(=O)(=O)O ; O=C(O)Cc1csc(NC(=O)C(c2ccccc2)S(=O)(=O)O)n1]

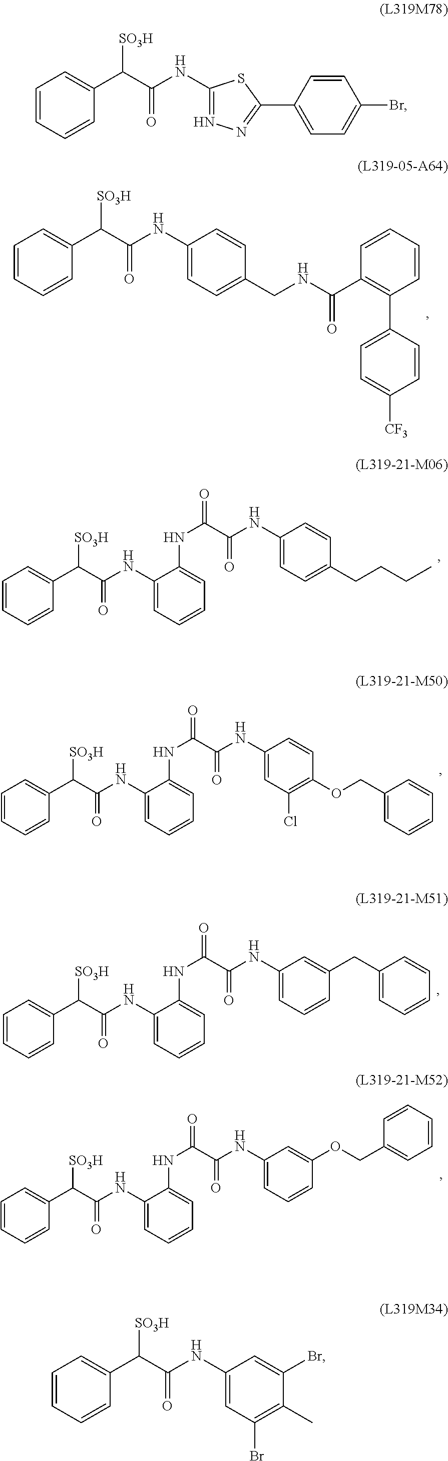

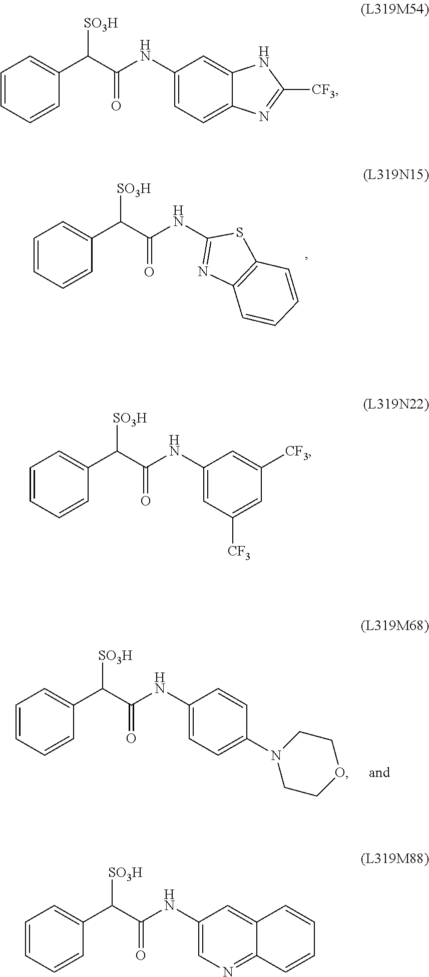 [SMILES: O=C(Nc1ccc2nc(C(F)(F)F)[nH]c2c1)C(c1ccccc1)S(=O)(=O)O ; O=C(Nc1nc2ccccc2s1)C(c1ccccc1)S(=O)(=O)O ; O=C(Nc1cc(C(F)(F)F)cc(C(F)(F)F)c1)C(c1ccccc1)S(=O)(=O)O ; O=C(Nc1ccc(N2CCOCC2)cc1)C(c1ccccc1)S(=O)(=O)O ; O=C(Nc1cnc2ccccc2c1)C(c1ccccc1)S(=O)(=O)O]

(as disclosed in U.S. Patent Application Publication No. 2018/0170862). The efficacy of the Nb72 nanobody disclosed in Simmons, et al. (2021)¹¹⁰ will also be examined. Further testing of the L319-21-M50 compound utilized in Example 2 may also be examined.

### GAA Inhibitors

In future studies, the efficacy of the GAA inhibitors of acarbose, miglitol, voglibose, castanospermine, miglustat, and 1-deoxynojirimycin with respect to treating PF in a subject having or at risk of having PF will be examined.

### GYS Inhibitors

In future studies, the efficacy of the GYS inhibitors will be examined. GYS inhibitors to be examined include, guaiacol,

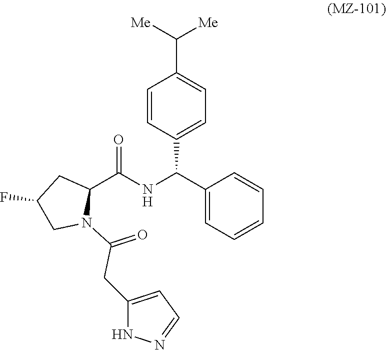 [SMILES: CC(C)c1ccc([C@@H](NC(=O)[C@@H]2C[C@@H](F)CN2C(=O)Cc2ccn[nH]2)c2ccccc2)cc1]

(as disclosed in Ullman et al. (2024)¹¹⁷), and compounds of the formula

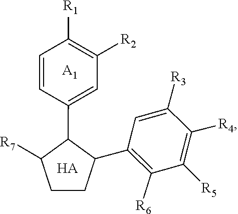

where HA is selected from

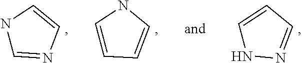

A₁ is selected from and

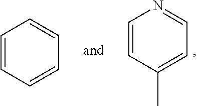

R₁ is selected from OH, H, CONH₂, F, Cl,

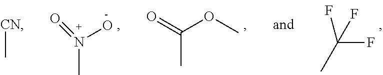

R₂ is selected from OCH₃, H, and Cl,
R₃ is selected from H and F,
R₄ is selected from H, OCH₃, and OH,
R₅ and R₆ are independently selected from H and OH, and
R₇ is selected from

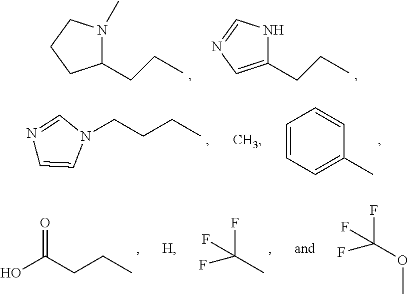

(as disclosed in Tang, et al. (2020)¹¹⁸. Specific compounds of such formula include

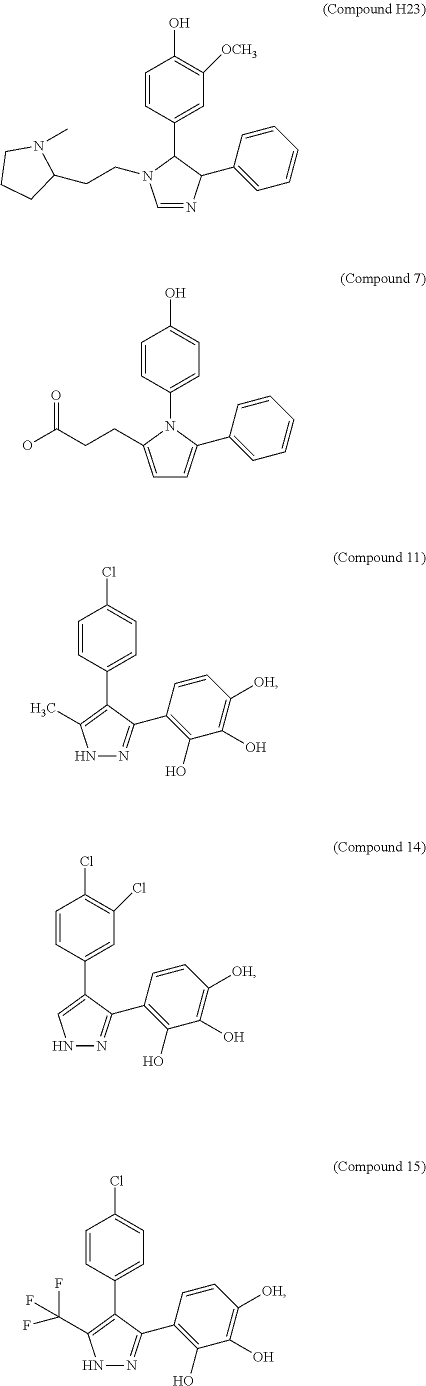 [SMILES: COc1cc(C2C(c3ccccc3)N=CN2CCC2CCCN2C)ccc1O ; O=C(O)CCc1ccc(-c2ccccc2)n1-c1ccc(O)cc1 ; Cc1[nH]nc(-c2ccc(O)c(O)c2O)c1-c1ccc(Cl)cc1 ; Oc1ccc(-c2n[nH]cc2-c2ccc(Cl)c(Cl)c2)c(O)c1O ; Oc1ccc(-c2n[nH]c(C(F)(F)F)c2-c2ccc(Cl)cc2)c(O)c1O]

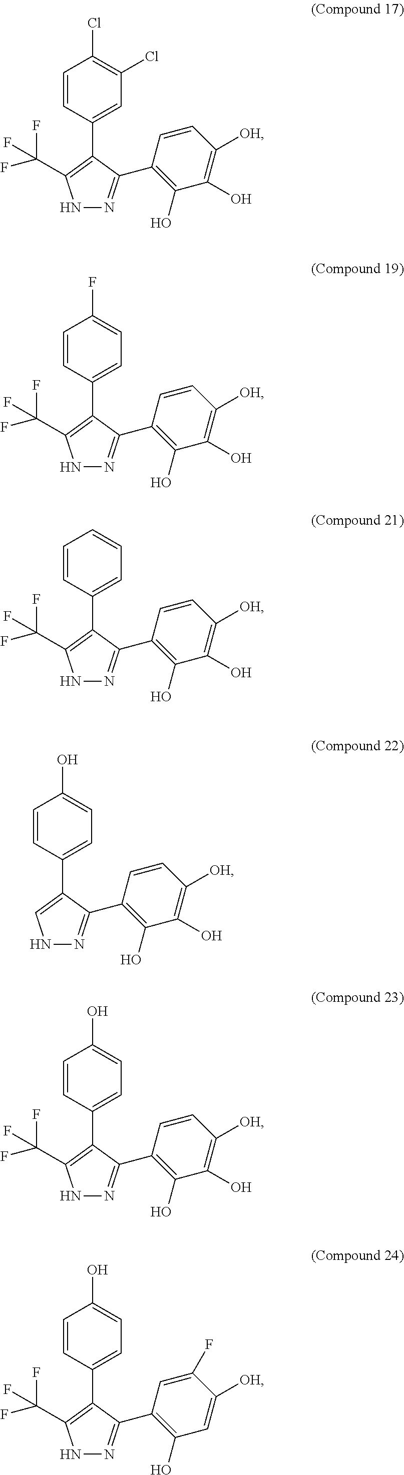 [SMILES: Oc1ccc(-c2n[nH]c(C(F)(F)F)c2-c2ccc(Cl)c(Cl)c2)c(O)c1O ; Oc1ccc(-c2n[nH]c(C(F)(F)F)c2-c2ccc(F)cc2)c(O)c1O ; Oc1ccc(-c2n[nH]c(C(F)(F)F)c2-c2ccccc2)c(O)c1O ; Oc1ccc(-c2c[nH]nc2-c2ccc(O)c(O)c2O)cc1 ; Oc1ccc(-c2c(-c3ccc(O)c(O)c3O)n[nH]c2C(F)(F)F)cc1 ; Oc1ccc(-c2c(-c3cc(F)c(O)cc3O)n[nH]c2C(F)(F)F)cc1]

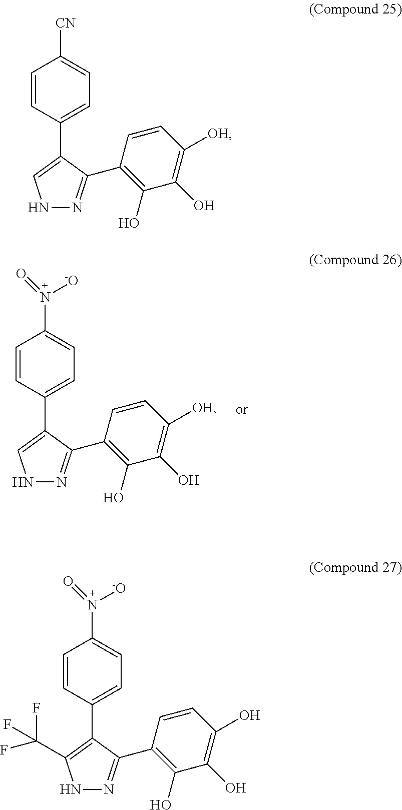

(as disclosed in Tang, et. al. (2020)¹¹⁸).

In future studies, the efficacy of the GP inhibitors will be examined. GP inhibitors to be examined include

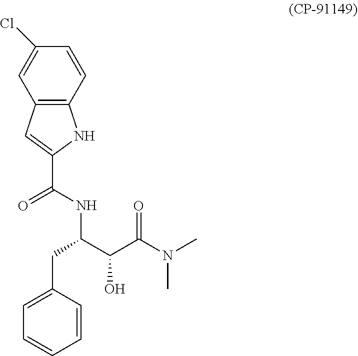 [SMILES: CN(C)C(=O)[C@H](O)[C@H](Cc1ccccc1)NC(=O)c1cc2cc(Cl)ccc2[nH]1]

(as disclosed in MedChem¹¹⁹)

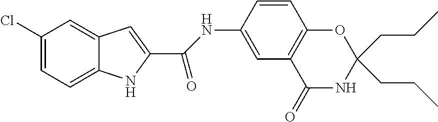 [SMILES: CCCC1(CCC)NC(=O)c2cc(NC(=O)c3cc4cc(Cl)ccc4[nH]3)ccc2O1]

(5-Chloro-N-phenyl-1H-indole-2-carboxamide derivative) (as disclosed in Huang, et al. (2023)¹²⁰), and

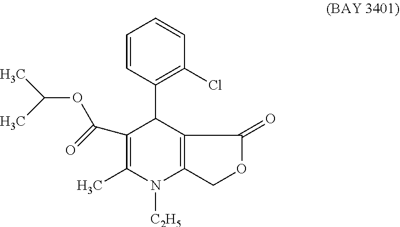 [SMILES: CCN1C(C)=C(C(=O)OC(C)C)C(c2ccccc2Cl)C2=C1COC2=O]

(as disclosed in Bergens, et al. (2000)¹²¹).

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Hwang, B., Lee, J. H. & Bang, D. Single-cell RNA sequencing technologies and bioinformatics pipelines. Exp Mol Med 50, 1-14, (2018).
2. Patel, A. P. et al. Single-cell RNA-seq highlights intratumoral heterogeneity in primary glioblastoma. Science 344, 1396-1401, (2014).
3. Shapira, G. & Shomron, N. Single-Cell Transcriptome Profiling. Methods Mol Biol 2243, 311-325, (2021).
4. Stuart, T. & Satija, R. Integrative single-cell analysis. Nat Rev Genet 20, 257-272, (2019).
5. Davis-Marcisak, E. F. et al. From bench to bedside: Single-cell analysis for cancer immunotherapy. Cancer Cell 39, 1062-1080, (2021).
6. He, P. et al. The changing mouse embryo transcriptome at whole tissue and single-cell resolution. Nature 583, 760-767, (2020).
7. Islam, S. et al. Quantitative single-cell RNA-seq with unique molecular identifiers. Nature methods 11, 163, (2014).
8. Olah, M. et al. Single cell RNA sequencing of human microglia uncovers a subset associated with Alzheimer's disease. Nat Commun 11, 6129, (2020).
9. Budnik, B., Levy, E., Harmange, G. & Slavov, N. SCoPE-MS: mass spectrometry of single mammalian cells quantifies proteome heterogeneity during cell differentiation. Genome Biol 19, 161, (2018).
10. Li, Z. et al. Single-cell lipidomics with high structural specificity by mass spectrometry. Nat Commun 12, 2869, (2021).
11. Shrestha, B. Single-Cell Metabolomics by Mass Spectrometry. Methods Mol Biol 2064, 1-8, (2020).
12. Seydel, C. Single-cell metabolomics hits its stride. Nat Methods 18, 1452-1456, (2021).
13. Taylor, M. J., Lukowski, J. K. & Anderton, C. R. Spatially Resolved Mass Spectrometry at the Single Cell: Recent Innovations in Proteomics and Metabolomics. J Am Soc Mass Spectrom 32, 872-894, (2021).
14. Conroy, L. R., Hawkinson, T. R., Young, L. E. A., Gentry, M. S. & Sun, R. C. Emerging roles of N-linked glycosylation in brain physiology and disorders. Trends Endocrinol Metab 32, 980-993, (2021).
15. Sun, R. C. et al. Brain glycogen serves as a critical glucosamine cache required for protein glycosylation. Cell Metab, (2021).
16. Stanley, P., Taniguchi, N. & Aebi, M. N-glycans. Essentials of Glycobiology [Internet]. 3rd edition, (2017).
17. Sun, R. C. et al. Nuclear glycogenolysis modulates histone acetylation in human non-small cell lung cancers. Cell metabolism 30, 903-916. e907, (2019).
18. Yan, A. & Lennarz, W. J. Unraveling the mechanism of protein N-glycosylation. Journal ofBiological Chemistry 280, 3121-3124, (2005).
19. Rudd, P. M. et al. Roles for glycosylation of cell surface receptors involved in cellular immune recognition. Journal of molecular biology 293, 351-366, (1999).
20. Gross, T. J. & Hunninghake, G. W. Idiopathic pulmonary fibrosis. New England Journal ofMedicine 345, 517-525, (2001).
21. Thannickal, V. J., Toews, G. B., White, E. S., Lynch Iii, J. P. & Martinez, F. J. Mechanisms of pulmonary fibrosis. Annual review of medicine 55, 395, (2004).
22. Hawkinson, T. R. & Sun, R. C. Matrix-Assisted Laser Desorption/Ionization Mass Spectrometry Imaging of Glycogen In Situ. Methods Mol Biol 2437, 215-228, (2022).
23. Cornett, D. S., Reyzer, M. L., Chaurand, P. & Caprioli, R. M. MALDI imaging mass spectrometry: molecular snapshots of biochemical systems. Nature methods 4, 828-833, (2007).
24. Stanback, A. E. et al. Regional N-glycan and lipid analysis from tissues using MALDI-mass spectrometry imaging. STAR protocols 2, 100304, (2021).
25. Prentice, B. M., Chumbley, C. W. & Caprioli, R. M. Absolute quantification of rifampicin by MALDI imaging mass spectrometry using multiple TOF/TOF events in a single laser shot. Journal of the American Society for Mass Spectrometry 28, 136-144, (2016).
26. Walch, A., Rauser, S., Deininger, S.-O. & Hofler, H. MALDI imaging mass spectrometry for direct tissue analysis: a new frontier for molecular histology. Histochemistry and cell biology 130, 421-434, (2008).
27. Li, L., Garden, R. W. & Sweedler, J. V. Single-cell MALDI: a new tool for direct peptide profiling. Trends in biotechnology 18, 151-160, (2000).
28. Andres, D. A., Young, L. E., Gentry, M. S. & Sun, R. C. Spatial profiling of gangliosides in mouse brain by mass spectrometry imaging. Journal of lipid research 61, 1537, (2020).
29. Prentice, B. M. in Mass Spectrometry Imaging of Small Molecules 3-19 (Springer, 2022).
30. Buck, A., Aichler, M., Huber, K. & Walch, A. In situ metabolomics in cancer by mass spectrometry imaging. Advances in cancer research 134, 117-132, (2017).
31. Powers, T. W., Holst, S., Wuhrer, M., Mehta, A. S. & Drake, R. R. Two-dimensional N-glycan distribution mapping of hepatocellular carcinoma tissues by MALDI-imaging mass spectrometry. Biomolecules 5, 2554-2572, (2015).
32. Powers, T. W. et al. MALDI imaging mass spectrometry profiling of N-glycans in formalin-fixed paraffin embedded clinical tissue blocks and tissue microarrays. PLoS One 9, e106255, (2014).
33. Clift, C. L., Drake, R. R., Mehta, A. & Angel, P. M. Multiplexed imaging mass spectrometry of the extracellular matrix using serial enzyme digests from formalin-fixed paraffin-embedded tissue sections. Anal Bioanal Chem 413, 2709-2719, (2021).
34. Drake, R. R., Powers, T. W., Norris-Caneda, K., Mehta, A. S. & Angel, P. M. In Situ Imaging of N-Glycans by MALDI Imaging Mass Spectrometry of Fresh or Formalin-Fixed Paraffin-Embedded Tissue. Curr Protoc Protein Sci 94, e68, (2018).
35. Young, L. E. et al. In situ mass spectrometry imaging reveals heterogeneous glycogen stores in human normal and cancerous tissues. EMBO Molecular Medicine, e16029.
36. Traag, V. A., Waltman, L. & van Eck, N. J. From Louvain to Leiden: guaranteeing well-connected communities. Sci Rep 9, 5233, (2019).
37. Becht, E. et al. Dimensionality reduction for visualizing single-cell data using UMAP. Nature Biotechnology 37, 38-44, (2019).
38. Wang, N. et al. Novel mechanism of the pericyte-myofibroblast transition in renal interstitial fibrosis: core fucosylation regulation. Scientific reports 7, 1-12, (2017).
39. Shen, N. et al. Inhibition of TGF-β1-receptor posttranslational core fucosylation attenuates rat renal interstitial fibrosis. Kidney international 84, 64-77, (2013).
40. Scanlin, T. F. & Glick, M. C. Terminal glycosylation in cystic fibrosis. Biochimica et Biophysica Acta (BBA)-Molecular Basis of Disease 1455, 241-253, (1999).
41. Sun, W. et al. Mechanisms of pulmonary fibrosis induced by core fucosylation in pericytes. The international journal of biochemistry & cell biology 88, 44-54, (2017).
42. Nakamura-Tsuruta, S. et al. Comparative analysis of carbohydrate-binding specificities of two anti-glycogen monoclonal antibodies using ELISA and surface plasmon resonance. Carbohydrate research 350, 49-54, (2012).
43. Sun, R. C. et al. Brain glycogen serves as a critical glucosamine cache required for protein glycosylation. Cell Metabolism 33, 1404-1417. e1409, (2021).
44. El Agha, E. et al. Two-way conversion between lipogenic and myogenic fibroblastic phenotypes marks the progression and resolution of lung fibrosis. Cell stem cell 20, 261-273. e263, (2017).
45. Young, L. E. A. et al. In situ mass spectrometry imaging reveals heterogeneous glycogen stores in human normal and cancerous tissues. EMBO Molecular Medicine n/a, e16029, (2022).
46. Li, S., Shi, J. & Tang, H. Animal models of drug-induced pulmonary fibrosis: an overview of molecular mechanisms and characteristics. Cell Biol Toxicol, (2021).
47. Liu, T., De Los Santos, F. G. & Phan, S. H. The Bleomycin Model of Pulmonary Fibrosis. Methods Mol Biol 1627, 27-42, (2017).
48. Gentry, M. S., Guinovart, J. J., Minassian, B. A., Roach, P. J. & Serratosa, J. M. Lafora disease offers a unique window into neuronal glycogen metabolism. Journal of Biological Chemistry 293, 7117-7125, (2018).
49. Gentry, M. S., Worby, C. A. & Dixon, J. E. Insights into Lafora disease: malin is an E3 ubiquitin ligase that ubiquitinates and promotes the degradation of laforin. Proc Natl Acad Sci USA 102, 8501-8506, (2005).
50. Brewer, M. K. & Gentry, M. S. Brain Glycogen Structure and Its Associated Proteins: Past, Present and Future. Adv Neurobiol 23, 17-81, (2019).
51. Brewer, M. K. et al. Targeting Pathogenic Lafora Bodies in Lafora Disease Using an Antibody-Enzyme Fusion. Cell Metabolism, (2019).
52. Gentry, M. S., Roma-Mateo, C. & Sanz, P. Laforin, a protein with many faces: glucan phosphatase, adapter protein, et alii. Febs J 280, 525-537, (2013).
53. Parker, M. W. et al. Fibrotic extracellular matrix activates a profibrotic positive feedback loop. The Journal of clinical investigation 124, (2014).
54. Ghosh, A. K. Factors involved in the regulation of type I collagen gene expression: implication in fibrosis. Experimental biology and medicine 227, 301-314, (2002).
55. Désogère, P. et al. Type I collagen-targeted PET probe for pulmonary fibrosis detection and staging in preclinical models. Science translational medicine 9, eaaf4696, (2017).
56. Angel, P. M. et al. Extracellular matrix imaging of breast tissue pathologies by MALDI-imaging mass spectrometry. PROTEOMICS—Clinical Applications 13, 1700152, (2019).
57. Angel, P. M. et al. Mapping extracellular matrix proteins in formalin-fixed, paraffin-embedded tissues by MALDI imaging mass spectrometry. J Proteome Res 17, 635-646, (2018).
58. Young, L. E. A. et al. Accurate and sensitive quantitation of glucose and glucose phosphates derived from storage carbohydrates by mass spectrometry. Carbohydrate Polymers 230, 115651, (2020).
59. Andres, D. A. et al. Improved workflow for mass spectrometry-based metabolomics analysis of the heart. Journal of Biological Chemistry, (2020).
60. Liu, Q. et al. Glycogen accumulation and phase separation drives liver tumor initiation. Cell 184, 5559-5576. e5519, (2021).
61. De Duve, C. The lysosome turns fifty. Nature cell biology 7, 847-849, (2005).
62. Maga, J. A. et al. Glycosylation-independent lysosomal targeting of acid α-glucosidase enhances muscle glycogen clearance in pompe mice. Journal of Biological Chemistry 288, 1428-1438, (2013).
63. Wisselaar, H. A., Kroos, M. A., Hermans, M., Van Beeumen, J. & Reuser, A. Structural and functional changes of lysosomal acid alpha-glucosidase during intracellular transport and maturation. Journal of Biological Chemistry 268, 2223-2231, (1993).
64. Reuser, A., Kroos, M., Elferink, R. O. & Tager, J. Defects in synthesis, phosphorylation, and maturation of acid alpha-glucosidase in glycogenosis type II. Journal of Biological Chemistry 260, 8336-8341, (1985).
65. Drake, R. R. et al. in Advances in cancer research Vol. 134 85-116 (Elsevier, 2017).
66. Reily, C., Stewart, T. J., Renfrow, M. B. & Novak, J. Glycosylation in health and disease. Nat Rev Nephrol 15, 346-366, (2019).
67. Haukedal, H. & Freude, K. K. Implications of Glycosylation in Alzheimer's Disease. Front Neurosci 14, 625348, (2020).
68. Raghunathan, R., Hogan, J. D., Labadorf, A., Myers, R. H. & Zaia, J. A glycomics and proteomics study of aging and Parkinson's disease in human brain. Sci Rep 10, 12804, (2020).
69. Štambuk, T. & Gornik, O. Protein Glycosylation in Diabetes. Adv Exp Med Biol 1325, 285-305, (2021).
70. Loaeza-Reyes, K. J. et al. An Overview of Glycosylation and its Impact on Cardiovascular Health and Disease. Front Mol Biosci 8, 751637, (2021).
71. Khan, T. et al. Revisiting Glycogen in Cancer: A Conspicuous and Targetable Enabler of Malignant Transformation. Front Oncol 10, 592455, (2020).
72. Pinho, S. S. & Reis, C. A. Glycosylation in cancer: mechanisms and clinical implications. Nat Rev Cancer 15, 540-555, (2015).
73. Saliba, A.-E., Westermann, A. J., Gorski, S. A. & Vogel, J. Single-cell RNA-seq: advances and future challenges. Nucleic Acids Res 42, 8845-8860, (2014).
74. Phillips, D. et al. Highly multiplexed phenotyping of immunoregulatory proteins in the tumor microenvironment by CODEX tissue imaging. Frontiers in Immunology 12, 687673, (2021).
75. Giuranno, L., lent, J., De Ruysscher, D. & Vooijs, M. A. Radiation-Induced Lung Injury (RILI). Front Oncol 9, 877, (2019).
76. Limper, A. H. Chemotherapy-induced lung disease. Clin Chest Med 25, 53-64, (2004).
77. Burnham, E. L., Janssen, W. J., Riches, D. W., Moss, M. & Downey, G. P. The fibroproliferative response in acute respiratory distress syndrome: mechanisms and clinical significance. Eur Respir J 43, 276-285, (2014).
78. López-Novoa, J. M. & Nieto, M. A. Inflammation and EMT: an alliance towards organ fibrosis and cancer progression. EMBO molecular medicine 1, 303-314, (2009).
79. George, P. M., Wells, A. U. & Jenkins, R. G. Pulmonary fibrosis and COVID-19: the potential role for antifibrotic therapy. Lancet Respir Med 8, 807-815, (2020).
80. Martinez, F. J. et al. Idiopathic pulmonary fibrosis. Nat Rev Dis Primers 3, 17074, (2017).
81. Ballester, B., Milara, J. & Cortijo, J. Mucins as a new frontier in pulmonary fibrosis. Journal of clinical medicine 8, 1447, (2019).
82. King Jr, T. E., Pardo, A. & Selman, M. Idiopathic pulmonary fibrosis. The Lancet 378, 1949-1961, (2011).
83. Raghu, G., Amatto, V. C., Behr, J. & Stowasser, S. Comorbidities in idiopathic pulmonary fibrosis patients: a systematic literature review. European Respiratory Journal 46, 1113-1130, (2015).
84. Raghu, G., Anstrom, K. J., King, T. E., Jr., Lasky, J. A. & Martinez, F. J. Prednisone, azathioprine, and N-acetylcysteine for pulmonary fibrosis. N Engl J Med 366, 1968-1977, (2012).
85. Walkley, S. U. Pathogenic mechanisms in lysosomal disease: a reappraisal of the role of the lysosome. Acta paediatrica 96, 26-32, (2007).
86. Conroy, L. R., Hawkinson, T. R., Young, L. E., Gentry, M. S. & Sun, R. C. Emerging roles of N-linked glycosylation in brain physiology and disorders. Trends in Endocrinology & Metabolism 32, 980-993, (2021).
87. Kakhlon, O., Escriba, P. V., Akman, H. O. & Weil, M. Editorial: Using Small Molecules to Treat Macromolecule Storage Disorders. Front Cell Dev Biol 8, 623613, (2020).
88. Kakhlon, O. et al. Guaiacol as a drug candidate for treating adult polyglucosan body disease. JCI Insight 3, (2018).
89. Birch, A. M. et al. Development of potent, orally active 1-substituted-3,4-dihydro-2-quinolone glycogen phosphorylase inhibitors. Bioorg Med Chem Lett 17, 394-399, (2007).
90. Martin, W. H. et al. Discovery of a human liver glycogen phosphorylase inhibitor that lowers blood glucose in vivo. Proc Natl Acad Sci USA 95, 1776-1781, (1998).
91. Tang, B. et al. Discovery and development of small-molecule inhibitors of glycogen synthase. Journal of medicinal chemistry 63, 3538-3551, (2020).
92. Milo, R., Jorgensen, P., Moran, U., Weber, G. & Springer, M. BioNumbers—the database of key numbers in molecular and cell biology. Nucleic Acids Res 38, D750-D753, (2010).
93. Moore, B. B. et al. Animal models of fibrotic lung disease. Am J Respir Cell Mol Biol 49, 167-179, (2013).
94. Zhang, H., Ma, J., Tang, K. & Huang, B. Beyond energy storage: roles of glycogen metabolism in health and disease. The FEBS Journal 288, 3772-3783, (2021).
95. Ganesh, S. et al. Targeted disruption of the Epm2a gene causes formation of Lafora inclusion bodies, neurodegeneration, ataxia, myoclonus epilepsy and impaired behavioral response in mice. Hum Mol Genet 11, 1251-1262, (2002).
96. Zhang, H. et al. Lkb1 inactivation drives lung cancer lineage switching governed by Polycomb Repressive Complex 2. Nat Commun 8, 14922, (2017).
97. Stanback, A. E. et al. Regional N-glycan and lipid analysis from tissues using MALDI-mass spectrometry imaging. STAR Protoc 2, 100304, (2021).
98. Oliphant, T. E. Python for scientific computing. Computing in Science & Engineering 9, 10-20, (2007).
99. Wolf, F. A., Angerer, P. & Theis, F. J. SCANPY: large-scale single-cell gene expression data analysis. Genome Biol 19, 15, (2018).
100. Conroy, L. R. et al. In situ analysis of N-linked Glycans as Potential Biomarkers of Clinical Course in Human Prostate Cancer. Molecular Cancer Research, (2021).
101. Wiederschain, G. Y. (Springer Nature BV, 2009).
102. Clift, C. L. et al. Collagen fiber regulation in human pediatric aortic valve development and disease. Scientific Reports 11, 1-17, (2021).
103. Angel, P. M. et al. Extracellular Matrix Imaging of Breast Tissue Pathologies by MALDI Imaging Mass Spectrometry. Proteomics Clinical Applications 13, e1700152. PMID: 30251340, (2019).
104. Angel, P. M. et al. Extracellular Matrix Alterations in Low Grade Lung Adenocarcinoma Compared to Normal Lung Tissue by Imaging Mass Spectrometry. Journal of Mass Spectrometry 55, e4450, (2019).
105. Angel, P. M. et al. Zonal regulation of collagen-type proteins and posttranslational modifications in prostatic benign and cancer tissues by imaging mass spectrometry. The Prostate 80, 1071-1086, (2020).
106. Angel, P. M. et al. Mapping Extracellular Matrix Proteins in Formalin-Fixed, Paraffin-embedded Tissues by MALDI Imaging Mass Spectrometry. Journal of Proteome Research 17, 635-646. PMID: 29161047, (2018).
107. Clift, C. L., Mehta, A. S., Drake, R. R. & Angel, P. M. Multiplexed Imaging Mass Spectrometry of Histological Staining, N-glycan and Extracellular Matrix from One Tissue Section: A Tool for Fibrosis Research. Methods in Molecular Biology In press, (2021).
108. Drake, R. R., Powers, T. W., Norris-Caneda, K., Mehta, A. S. & Angel, P. M. In Situ Imaging of N-Glycans by MALDI Imaging Mass Spectrometry of Fresh or Formalin-Fixed Paraffin-Embedded Tissue. Current Protocols in Protein Science 94, e68. PMID: 30074304, (2018).
109. Saeed, A. I. et al. TM4 Microarray Software Suite. Methods in Enzymology 411, 134193. PMID: 16939790, (2006).
110. Simmons, Z. R., Sharma, S., Wayne, J., Li, S., Vander Kooi, C. W., and Gentry, M. S. “Generation and characterization of a laforin nanobody inhibitor,” Clinical Biochemistry 93, 80-89, (2021).
111. U.S. Pat. No. 10,532,977 for “Small molecule inhibitors of protein tyrosine phosphatases and uses thereof.”
112. U.S. Patent Application Publication No. 2018/0170862 for “Protein tyrosine phosphatases or shp2 inhibitors and uses thereof.”
113. U.S. Patent Application Publication No. 2022/0151581 for “Methods for treating idiopathic pulmonary fibrosis.”
114. U.S. Patent Application Publication No. 2021/0179680 for “Polypeptide, derivatives thereof, and application thereof in preparation of drugs having resistance to pulmonary fibrosis.”
115. U.S. Patent Application Publication No. 2022/0135643 for “Soluble thy-1 compositions and use thereof to treat or reverse fibrosis.”
116. U.S. Patent Application Publication No. 2022/0000772 for “Specially formulated compositions of inhaled nintedanib and nintedanib salts.”
117. Ullman, et al., “Small-Molecule Inhibition of Glycogen Synthase 1 for the Treatment of Pompe Disease and Other Glycogen Storage Disorders” Science Translational Medicine Vol. 16, Issue 730 (2024).
118. Tang, B. et al., “Discovery and Development of Small-Molecule Inhibitors of Glycogen Synthase” Journal of medicinal chemistry 63, 3538-3551 (2020).
119. MedChem Express, CP-91149 Product Data Sheet, available at https://file.medchemexpress.com/batch_PDF/HY-13525/CP-91149-DataSheet-MedChemExpress.pdf (accessed Feb. 14, 2024).
120. Huang, et al., “A Novel 5-Chloro-N-phenyl-1H-indole-2-carboxamide Derivative as Brain-Type Glycogen Phosphorylase Inhibitor: Validation of Target PYGB” Molecules 28(4): 1697 (2023).
121. Bergans, et al., “Molecular Mode of Inhibition of Glycogenolysis in Rat Liver by the Dihydropyridine Derivative, BAY 3401: Inhibition and Inactivation of Glycgogen Phosphorylase by an Activated Metabolite” Diabetes 40(9): 1419-1426 (2000).
122. Conroy, et al., “Spatial Metabolomics Reveals Glycogen as an Actionable Target for Pulmonary Fibrosis” Nature Communications 14, Article number: 2759 (2023).

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A method of treating pulmonary fibrosis (PF) in a subject in need thereof, comprising administering to the subject an effective amount of a compound selected from the group consisting of a glycogen phosphatase inhibitor, an acid alpha-glucosidase (GAA) inhibitor, a glycogen synthase (GYS) inhibitor, a glycogen phosphorylase (GP) inhibitor, and combinations thereof, or a pharmaceutically acceptable salt thereof.

2. The method of claim 1, wherein the compound is the glycogen phosphatase inhibitor.

3. The method of claim 2, wherein the glycogen phosphatase inhibitor is a laforin inhibitor.

4. The method of claim 3, wherein the laforin inhibitor is of the formula selected from the group consisting of

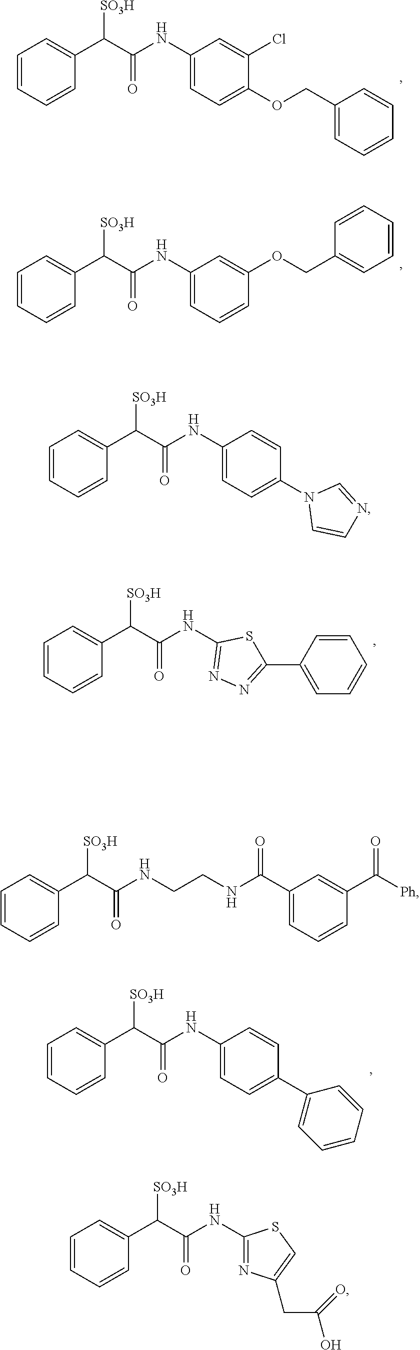

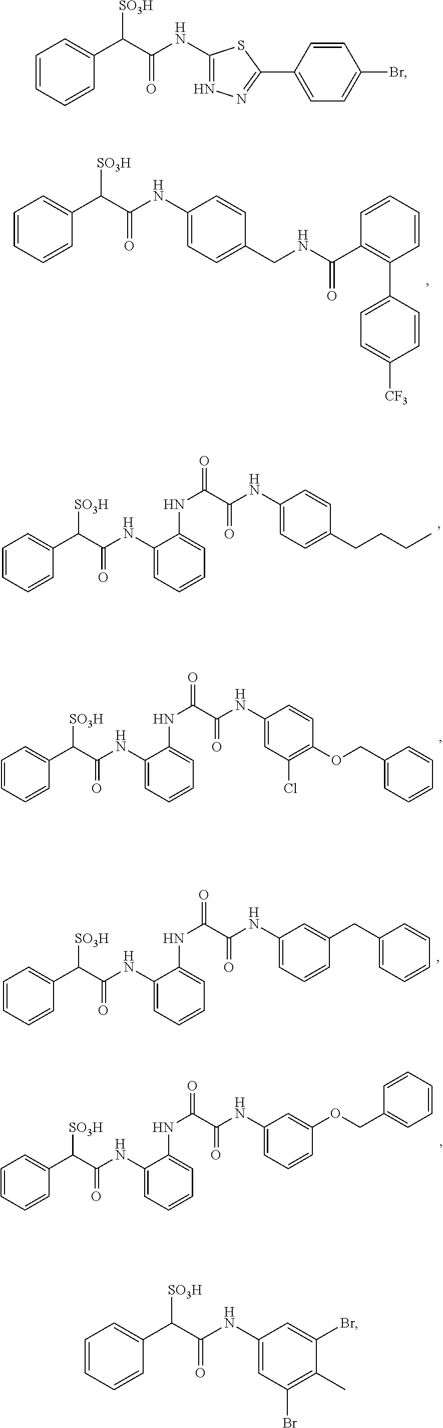

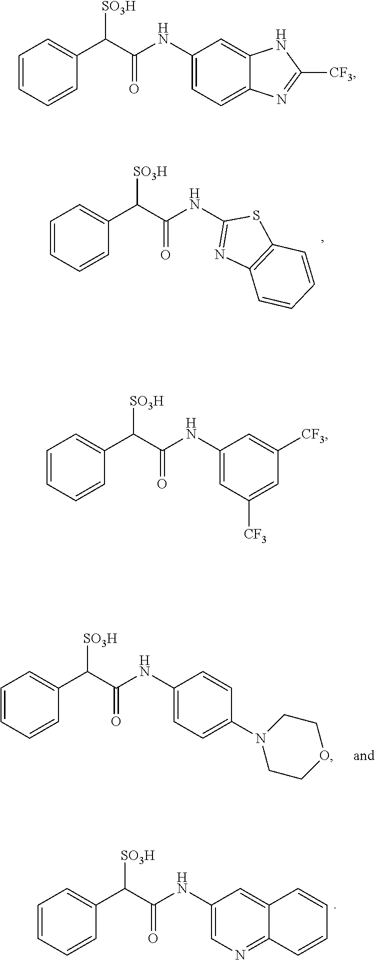 [SMILES: O=C(Nc1ccc2nc(C(F)(F)F)[nH]c2c1)C(c1ccccc1)S(=O)(=O)O ; O=C(Nc1nc2ccccc2s1)C(c1ccccc1)S(=O)(=O)O ; O=C(Nc1cc(C(F)(F)F)cc(C(F)(F)F)c1)C(c1ccccc1)S(=O)(=O)O ; O=C(Nc1ccc(N2CCOCC2)cc1)C(c1ccccc1)S(=O)(=O)O ; O=C(Nc1cnc2ccccc2c1)C(c1ccccc1)S(=O)(=O)O]

5. The method of claim 3, wherein the laforin inhibitor is of the formula of

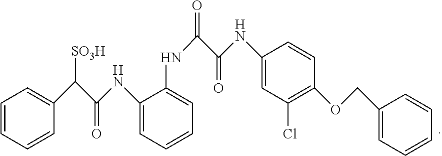 [SMILES: O=C(Nc1ccc(OCc2ccccc2)c(Cl)c1)C(=O)Nc1ccccc1NC(=O)C(c1ccccc1)S(=O)(=O)O]

6. The method of claim 3, wherein the laforin inhibitor is a nanobody comprising the sequence of SEQ TD NO: 1, SEQ TD NO: 2, and SEQ TD NO: 3.

7. The method of claim 3, wherein the laforin inhibitor is a nanobody comprising the sequence of SEQ ID NO: 4.

8. The method of claim 1, wherein the compound is the GAA inhibitor.

9. The method of claim 1, wherein the GAA inhibitor is selected from the group consisting of acarbose, miglitol, voglibose, castanospermine, miglustat, and 1-deoxynojirimycin.

10. The method of claim 1, wherein the compound is the GYS inhibitor.

11. The method of claim 10, wherein the GYS inhibitor is of the formula of: 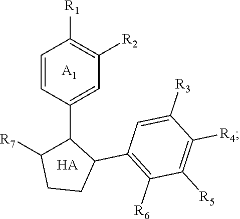
wherein HA is selected from the group consisting of 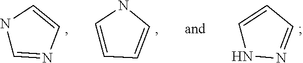
wherein A₁ is selected from the group consisting of 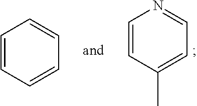
wherein R₁ is selected from the group consisting of OH, H, CONH₂, F, Cl, 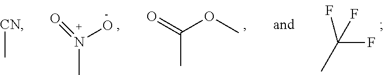
wherein R₂ is selected from the group consisting of OCH₃, H, and Cl;
wherein R₃ is selected from the group consisting of H and F;
wherein R₄ is selected from the group consisting of H, OCH₃, and OH;
wherein R₅ and R₆ are independently selected from the group consisting of H and OH; and
wherein R₇ is selected from the group consisting of

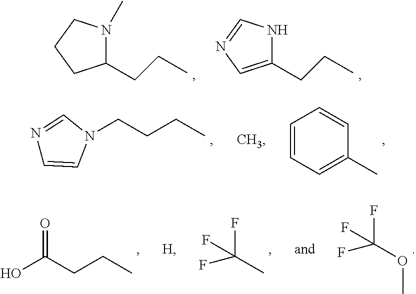

11. The method of claim 10, wherein the compound is of the formula selected from the group consisting of

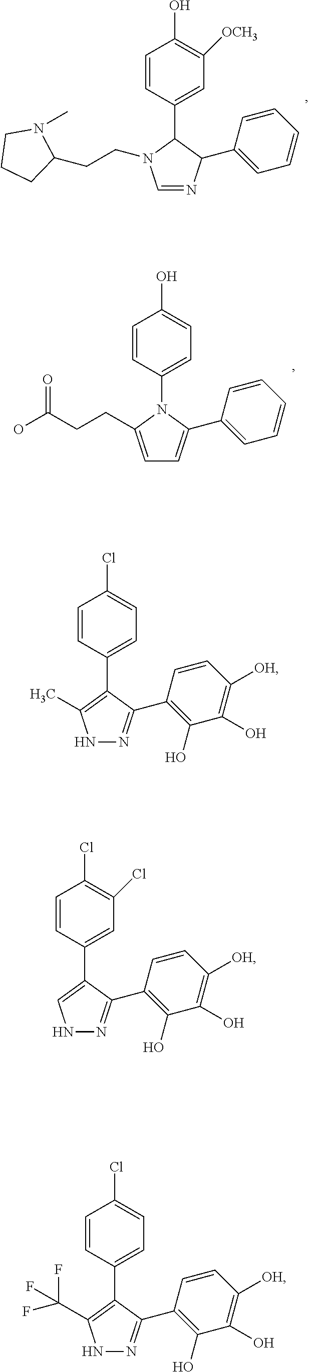 [SMILES: COc1cc(C2C(c3ccccc3)N=CN2CCC2CCCN2C)ccc1O ; O=C(O)CCc1ccc(-c2ccccc2)n1-c1ccc(O)cc1 ; Cc1[nH]nc(-c2ccc(O)c(O)c2O)c1-c1ccc(Cl)cc1 ; Oc1ccc(-c2n[nH]cc2-c2ccc(Cl)c(Cl)c2)c(O)c1O ; Oc1ccc(-c2n[nH]c(C(F)(F)F)c2-c2ccc(Cl)cc2)c(O)c1O]

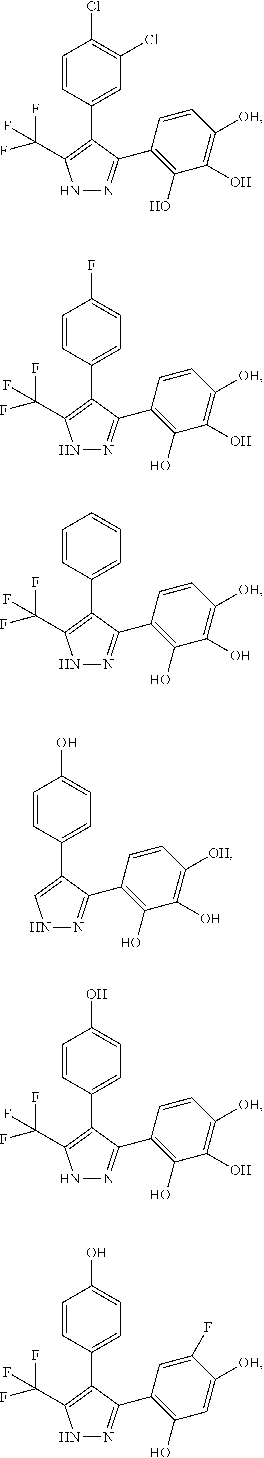 [SMILES: Oc1ccc(-c2n[nH]c(C(F)(F)F)c2-c2ccc(Cl)c(Cl)c2)c(O)c1O ; Oc1ccc(-c2n[nH]c(C(F)(F)F)c2-c2ccc(F)cc2)c(O)c1O ; Oc1ccc(-c2n[nH]c(C(F)(F)F)c2-c2ccccc2)c(O)c1O ; Oc1ccc(-c2c[nH]nc2-c2ccc(O)c(O)c2O)cc1 ; Oc1ccc(-c2c(-c3ccc(O)c(O)c3O)n[nH]c2C(F)(F)F)cc1 ; Oc1ccc(-c2c(-c3cc(F)c(O)cc3O)n[nH]c2C(F)(F)F)cc1]

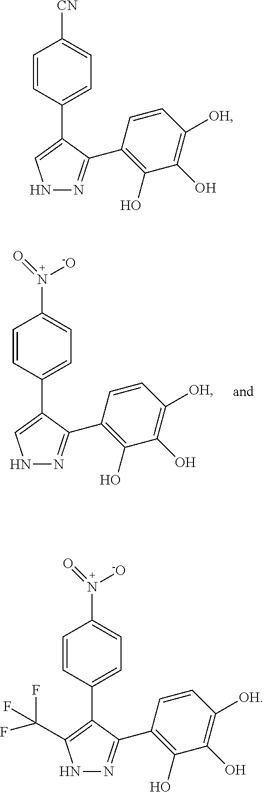

13. The method of claim 10, wherein the GYS inhibitor is of the formula of

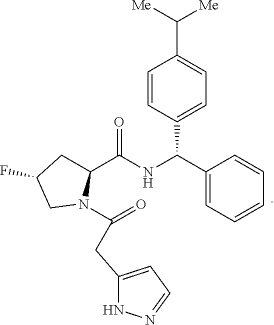 [SMILES: CC(C)c1ccc([C@@H](NC(=O)[C@@H]2C[C@@H](F)CN2C(=O)Cc2ccn[nH]2)c2ccccc2)cc1]

14. The method of claim 10, wherein the GYS inhibitor is guaiacol.

15. The method of claim 1, wherein the compound is the GP inhibitor.

16. The method of claim 14, wherein the GP inhibitor is of the formula selected from the group consisting of

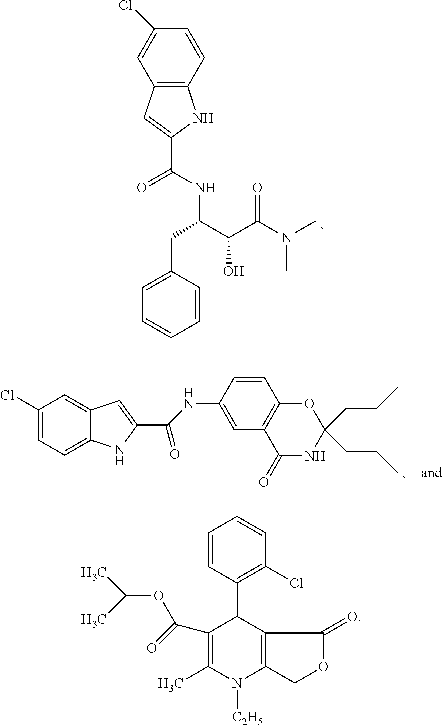

17. The method of claim 1, wherein the subject is identified as being at risk for PF.

18. The method of claim 1, wherein the subject is identified as having PF.

19. The method of claim 1, wherein the subject has a history of at least one of smoking and a family history of PF.


SEQUENCE LISTING (4 sequences)

SEQ ID NO: 1 - protein, 5 aa, synthetic construct
INAMA

SEQ ID NO: 2 - protein, 15 aa, synthetic construct
HISSDNTNYADSVKG

SEQ ID NO: 3 - protein, 4 aa, synthetic construct
VATW

SEQ ID NO: 4 - protein, 129 aa, synthetic construct
QVQLQESGGGLVQPGGSLRLSCAASGNIFSINAMAWYRQAPGKLREYVAHISSDNTNYADSVKGRFTISRNNVGNMAYLQMNSLKPEDTAVYYCTSVATWGQGTQVTVSSAAAYPYDVPDYGSHHHHHH